SEQUENCE LISTING (47 sequences)

SEQ ID NO: 1 - protein, 22 aa, Homo sapiens
RRRPKGRGKRRREKQRPTDCHL

SEQ ID NO: 2 - protein, 30 aa, Homo sapiens
GRREEKVGKKEKIGKKKRQKKRKAAQKRKN

SEQ ID NO: 3 - protein, 205 aa, Homo sapiens
CSGEGLQIPTLSPAPDCSQPLDVILLLDGSSSFPASYFDEMKSFAKAFISKANIGPRLTQVSVLQYGSITTIDVPWNVVPEKAHLLSLVDVMQREGGPSQIGDALGFAVRYLTSEMHGARPGASKAVVILVTDVSVDSVDAAADAARSNRVTVFPIGIGDRYDAAQLRILAGPAGDSNVVKLQRIEDLPTMVTLGNSFLHKLCSG

SEQ ID NO: 4 - protein, 170 aa, Homo sapiens
MPVMRLFPCFLQLLAGLALPAVPPQQWALSAGNGSSEVEVVPFQEVWGRSYCRALERLVDVVSEYPSEVEHMFSPSCVSLLRCTGCCGDENLHCVPVETANVTMQLLKIRSGDRPSYVELTFSQHVRCECRPLREKMKPERRRPKGRGKRRREKQRPTDCHLCGDAVPRR

SEQ ID NO: 5 - protein, 30 aa, Homo sapiens
RRRPKGRGKRRREKQRPTDCHLCGDAVPRR

SEQ ID NO: 6 - protein, 18 aa, Homo sapiens
RRPKGRGKRRREKQRPTD

SEQ ID NO: 7 - protein, 15 aa, Homo sapiens
RRRPKGRGKRRREKQ

SEQ ID NO: 8 - protein, 8 aa, Homo sapiens
GKRRREKQ

SEQ ID NO: 9 - protein, 8 aa, Homo sapiens
RRRPKGRG

SEQ ID NO: 10 - protein, 21 aa, Homo sapiens
RRKTKGKRKRSRNSQTEEPHP

SEQ ID NO: 11 - protein, 222 aa, Homo sapiens
CQEPGGLVVPPTDAPVSPTTLYVEDISEPPLHDFYCSRLLDLVFLLDGSSRLSEAEFEVLKAFVVDMMERLRISQKWVRVAVVEYHDGSHAYIGLKDRKRPSELRRIASQVKYAGSQVASTSEVLKYTLFQIFSKIDRPEASRITLLLMASQEPQRMSRNFVRYVQGLKKKKVIVIPVGIGPHANLKQIRLIEKQAPENKAFVLSSVDELEQQRDEIVSYLC

SEQ ID NO: 12 - protein, 98 aa, Homo sapiens
KPVSLSYRCPCRFFESHVARANVKHLKILNTPNCALQIVARLKNNNRQVCIDPKLKWIQEYLEKALNKGRREEKVGKKEKIGKKKRQKKRKAAQKRKN

SEQ ID NO: 13 - protein, 2813 aa, Homo sapiens
MIPARFAGVLLALALILPGTLCAEGTRGRSSTARCSLFGSDFVNTFDGSMYSFAGYCSYLLAGGCQKRSFSIIGDFQNGKRVSLSVYLGEFFDIHLFVNGTVTQGDQRVSMPYASKGLYLETEAGYYKLSGEAYGFVARIDGSGNFQVLLSDRYFNKTCGLCGNFNIFAEDDFMTQEGTLTSDPYDFANSWALSSGEQWCERASPPSSSCNISSGEMQKGLWEQCQLLKSTSVFARCHPLVDPEPFVALCEKTLCECAGGLECACPALLEYARTCAQEGMVLYGWTDHSACSPVCPAGMEYRQCVSPCARTCQSLHINEMCQERCVDGCSCPEGQLLDEGLCVESTECPCVHSGKRYPPGTSLSRDCNTCICRNSQWICSNEECPGECLVTGQSHFKSFDNRYFTFSGICQYLLARDCQDHSFSIVIETVQCADDRDAVCTRSVTVRLPGLHNSLVKLKHGAGVAMDGQDVQLPLLKGDLRIQHTVTASVRLSYGEDLQMDWDGRGRLLVKLSPVYAGKTCGLCGNYNGNQGDDFLTPSGLAEPRVEDFGNAWKLHGDCQDLQKQHSDPCALNPRMTRFSEEACAVLTSPTFEACHRAVSPLPYLRNCRYDVCSCSDGRECLCGALASYAAACAGRGVRVAWREPGRCELNCPKGQVYLQCGTPCNLTCRSLSYPDEECNEACLEGCFCPPGLYMDERGDCVPKAQCPCYYDGEIFQPEDIFSDHHTMCYCEDGFMHCTMSGVPGSLLPDAVLSSPLSHRSKRSLSCRPPMVKLVCPADNLRAEGLECTKTCQNYDLECMSMGCVSGCLCPPGMVRHENRCVALERCPCFHQGKEYAPGETVKIGCNTCVCRDRKWNCTDHVCDATCSTIGMAHYLTFDGLKYLFPGECQYVLVQDYCGSNPGTFRILVGNKGCSHPSVKCKKRVTILVEGGEIELFDGEVNVKRPMKDETHFEVVESGRYIILLLGKALSVVWDRHLSISVVLKQTYQEKVCGLCGNFDGIQNNDLTSSNLQVEEDPVDFGNSWKVSSQCADTRKVPLDSSPATCHNNIMKQTMVDSSCRILTSDVFQDCNKLVDPEPYLDVCIYDTCSCESIGDCACFCDTIAAYAHVCAQHGKVVTWRTATLCPQSCEERNLRENGYECEWRYNSCAPACQVTCQHPEPLACPVQCVEGCHAHCPPGKILDELLQTCVDPEDCPVCEVAGRRFASGKKVTLNPSDPEHCQICHCDVVNLTCEACQEPGGLVVPPTDAPVSPTTLYVEDISEPPLHDFYCSRLLDLVFLLDGSSRLSEAEFEVLKAFVVDMMERLRISQKWVRVAVVEYHDGSHAYIGLKDRKRPSELRRIASQVKYAGSQVASTSEVLKYTLFQIFSKIDRPEASRITLLLMASQEPQRMSRNFVRYVQGLKKKKVIVIPVGIGPHANLKQIRLIEKQAPENKAFVLSSVDELEQQRDEIVSYLCDLAPEAPPPTLPPDMAQVTVGPGLLGVSTLGPKRNSMVLDVAFVLEGSDKIGEADFNRSKEFMEEVIQRMDVGQDSIHVTVLQYSYMVTVEYPFSEAQSKGDILQRVREIRYQGGNRTNTGLALRYLSDHSFLVSQGDREQAPNLVYMVTGNPASDEIKRLPGDIQVVPIGVGPNANVQELERIGWPNAPILIQDFETLPREAPDLVLQRCCSGEGLQIPTLSPAPDCSQPLDVILLLDGSSSFPASYFDEMKSFAKAFISKANIGPRLTQVSVLQYGSITTIDVPWNVVPEKAHLLSLVDVMQREGGPSQIGDALGFAVRYLTSEMHGARPGASKAVVILVTDVSVDSVDAAADAARSNRVTVFPIGIGDRYDAAQLRILAGPAGDSNVVKLQRIEDLPTMVTLGNSFLHKLCSGFVRICMDEDGNEKRPGDVWTLPDQCHTVTCQPDGQTLLKSHRVNCDRGLRPSCPNSQSPVKVEETCGCRWTCPCVCTGSSTRHIVTFDGQNFKLTGSCSYVLFQNKEQDLEVILHNGACSPGARQGCMKSIEVKHSALSVELHSDMEVTVNGRLVSVPYVGGNMEVNVYGAIMHEVRFNHLGHIFTFTPQNNEFQLQLSPKTFASKTYGLCGICDENGANDFMLRDGTVTTDWKTLVQEWTVQRPGQTCQPILEEQCLVPDSSHCQVLLLPLFAECHKVLAPATFYAICQQDSCHQEQVCEVIASYAHLCRTNGVCVDWRTPDFCAMSCPPSLVYNHCEHGCPRHCDGNVSSCGDHPSEGCFCPPDKVMLEGSCVPEEACTQCIGEDGVQHQFLEAWVPDHQPCQICTCLSGRKVNCTTQPCPTAKAPTCGLCEVARLRQNADQCCPEYECVCDPVSCDLPPVPHCERGLQPTLTNPGECRPNFTCACRKEECKRVSPPSCPPHRLPTLRKTQCCDEYECACNCVNSTVSCPLGYLASTATNDCGCTTTTCLPDKVCVHRSTIYPVGQFWEEGCDVCTCTDMEDAVMGLRVAQCSQKPCEDSCRSGFTYVLHEGECCGRCLPSACEVVTGSPRGDSQSSWKSVGSQWASPENPCLINECVRVKEEVFIQQRNVSCPQLEVPVCPSGFQLSCKTSACCPSCRCERMEACMLNGTVIGPGKTVMIDVCTTCRCMVQVGVISGFKLECRKTTCNPCPLGYKEENNTGECCGRCLPTACTIQLRGGQIMTLKRDETLQDGCDTHFCKVNERGEYFWEKRVTGCPPFDEHKCLAEGGKIMKIPGTCCDTCEEPECNDITARLQYVKVGSCKSEVEVDIHYCQGKCASKAMYSIDINDVQDQCSCCSPTRTEPMQVALHCTNGSVVYHEVLNAMECKCSPRKCSK

SEQ ID NO: 14 - protein, 6 aa, Homo sapiens
TKKLRT

SEQ ID NO: 15 - protein, 343 aa, Homo sapiens
GPFQQRGLFDFMLEDEASGIGPEVPDDRDFEPSLGPVCPFRCQCHLRVVQCSDLGLDKVPKDLPPDTTLLDLQNNKITEIKDGDFKNLKNLHALILVNNKISKVSPGAFTPLVKLERLYLSKNQLKELPEKMPKTLQELRAHENEITKVRKVTFNGLNQMIVIELGTNPLKSSGIENGAFQGMKKLSYIRIADTNITSIPQGLPPSLTELHLDGNKISRVDAASLKGLNNLAKLGLSFNSISAVDNGSLANTPHLRELHLDNNKLTRVPGGLAEHKYIQVVYLHNNNISVVGSSDFCPPGHNTKKASYSGVSLFSNPVQYWEIQPSTFRCVYVRSAIQLGNYK

SEQ ID NO: 16 - protein, 115 aa, Homo sapiens
MNWVNVISDLKKIEDLIQSMHIDATLYTESDVHPSCKVTAMKCFLLELQVISLESGDASIHDTVENLIILANNSLSSNGNVTESGCKECEELEEKNIKEFLQSFVHIVQMFINTS

SEQ ID NO: 17 - protein, 115 aa, Mus musculus
MNWIDVRYDLEKIESLIQSIHIDTTLYTDSDFHPSCKVTAMNCFLLELQVILHEYSNMTLNETVRNVLYLANSTLSSNKNVAESGCKECEELEEKTFTEFLQSFIRIVQMFINTS

SEQ ID NO: 18 - protein, 132 aa, Homo sapiens
MQDRHMIRMRQLIDIVDQLKNYVNDLVPEFLPAPEDVETNCEWSAFSCFQKAQLKSANTGNNERIINVSIKKLKRKPPSTNAGRRQKHRLTCPSCDSYEKKPPKEFLERFKSLLQKMIHQHLSSRTHGSEDS

SEQ ID NO: 19 - protein, 130 aa, Mus musculus
MHKSSPQGPDRLLIRLRHLIDIVEQLKIYENDLDPELLSAPQDVKGHCEHAAFACFQKAKLKPSNPGNNKTFIIDLVAQLRRRLPARRGGKKQKHIAKCPSCDSYEKRTPKEFLERLKWLLQKMIHQHLS

SEQ ID NO: 20 - protein, 197 aa, Homo sapiens
RNLPVATPDPGMFPCLHHSQNLLRAVSNMLQKARQTLEFYPCTSEEIDHEDITKDKTSTVEACLPLELTKNESCLNSRETSFITNGSCLASRKTSFMMALCLSSIYEDLKMYQVEFKTMNAKLLMDPKRQIFLDQNMLAVIDELMQALNFNSETVPQKSSLEEPDFYKTKIKLCILLHAFRIRAVTIDRVMSYLNAS

SEQ ID NO: 21 - protein, 306 aa, Homo sapiens
IWELKKDVYVVELDWYPDAPGEMVVLTCDTPEEDGITWTLDQSSEVLGSGKTLTIQVKEFGDAGQYTCHKGGEVLSHSLLLLHKKEDGIWSTDILKDQKEPKNKTFLRCEAKNYSGRFTCWWLTTISTDLTFSVKSSRGSSDPQGVTCGAATLSAERVRGDNKEYEYSVECQEDSACPAAEESLPIEVMVDAVHKLKYENYTSSFFIRDIIKPDPPKNLQLKPLKNSRQVEVSWEYPDTWSTPHSYFSLTFCVQVQGKSKREKKDRVFTDKTSATVICRKNASISVRAQDRYYSSSWSEWASVPCS

SEQ ID NO: 22 - protein, 193 aa, Mus musculus
RVIPVSGPARCLSQSRNLLKTTDDMVKTAREKLKHYSCTAEDIDHEDITRDQTSTLKTCLPLELHKNESCLATRETSSTTRGSCLPPQKTSLMMTLCLGSIYEDLKMYQTEFQAINAALQNHNHQQIILDKGMLVAIDELMQSLNHNGETLRQKPPVGEADPYRVKMKLCILLHAFSTRVVTINRVMGYLSSA

SEQ ID NO: 23 - protein, 313 aa, Mus musculus
MWELEKDVYVVEVDWTPDAPGETVNLTCDTPEEDDITWTSDQRHGVIGSGKTLTITVKEFLDAGQYTCHKGGETLSHSHLLLHKKENGIWSTEILKNFKNKTFLKCEAPNYSGRFTCSWLVQRNMDLKFNIKSSSSSPDSRAVTCGMASLSAEKVTLDQRDYEKYSVSCQEDVTCPTAEETLPIELALEARQQNKYENYSTSFFIRDIIKPDPPKNLQMKPLKNSQVEVSWEYPDSWSTPHSYFSLKFFVRIQRKKEKMKETEEGCNQKGAFLVEKTSTEVQCKGGNVCVQAQDRYYNSSCSKWACVPCRVRS

SEQ ID NO: 24 - protein, 69 aa, Homo sapiens
APMGSDPPTACCFSYTARKLPHNFVVDYYETSSLCSQPAVVFQTKRGKQVCADPSESWVQEYVYDLELN

SEQ ID NO: 25 - protein, 69 aa, Mus musculus
APMGSDPPTSCCFSYTSRQLHRSFVMDYYETSSLCSKPAVVFLTKRGRQICANPSEPWVTEYMSDLELN

SEQ ID NO: 26 - protein, 111 aa, Homo sapiens
SDGGAQDCCLKYSQRKIPAKVVRSYRKQEPSLGCSIPAILFLPRKRSQAELCADPKELWVQQLMQHLDKTPSPQKPAQGCRKDRGASKTGKKGKGSKGCRKTERSQTPKGP

SEQ ID NO: 27 - protein, 110 aa, Mus musculus
SDGGGQDCCLKYSQKKIPYSIVRGYRKQEPSLGCPIPAILFLPRKHSKPELCANPEEGWVQNLMRRLDQPPAPGKQSPGCRKNRGTSKSGKKGKGSKGCKRTEQTQPSRG

SEQ ID NO: 28 - protein, 103 aa, Homo sapiens
TPVVRKGRCSCISTNQGTIHLQSLKDLKQFAPSPSCEKIEIIATLKNGVQTCLNPDSADVKELIKKWEKQVSQKKKQKNGKKHQKKKVLKVRKSQRSRQKKTT

SEQ ID NO: 29 - protein, 105 aa, Mus musculus
TLVIRNARCSCISTSRGTIHYKSLKDLKQFAPSPNCNKTEIIATLKNGDQTCLDPDSANVKKLMKEWEKKINQKKKQKRGKKHQKNMKNRKPKTPQSRRRSRKTT

SEQ ID NO: 30 - protein, 77 aa, Homo sapiens
VPLSRTVRCTCISISNQPVNPRSLEKLEIIPASQFCPRVEIIATMKKKGEKRCLNPESKAIKNLLKAVSKERSKRSP

SEQ ID NO: 31 - protein, 77 aa, Mus musculus
IPLARTVRCNCIHIDDGPVRMRAIGKLEIIPASLSCPRVEIIATMKKNDEQRCLNPESKTIKNLMKAFSQKRSKRAP

SEQ ID NO: 32 - protein, 116 aa, Homo sapiens
AHYNTEILKSIDNEWRKTQCMPREVCIDVGKEFGVATNTFFKPPCVSVYRCGGCCNSEGLQCMNTSTSYLSKTLFEITVPLSQGPKPVTISFANHTSCRCMSKLDVYRQVHSIIRR

SEQ ID NO: 33 - protein, 125 aa, Mus musculus
TEETIKFAAAHYNTEILKSIDNEWRKTQCMPREVCIDVGKEFGVATNTFFKPPCVSVYRCGGCCNSEGLQCMNTSTSYLSKTLFEITVPLSQGPKPVTISFANHTSCRCMSKLDVYRQVHSIIRR

SEQ ID NO: 34 - protein, 196 aa, Homo sapiens
CSQPLDVILLLDGSSSFPASYFDEMKSFAKAFISKANIGPRLTQVSVLQYGSITTIDVPWNVVPEKAHLLSLVDVMQREGGPSQIGDALGFAVRYLTSEMHGARPGASKAVVILVTDVSVDSVDAAADAARSNRVTVFPIGIGDRYDAAQLRILAGPAGDSNVVKLQRIEDLPTMVTLGNSFLHKLCSGFVRICTG

SEQ ID NO: 35 - protein, 148 aa, Mus musculus
PTSSSTSSSTAEAQQQQQQQQQQQQHLEQLLMDLQELLSRMENYRNLKLPRMLTFKFYLPKQATELKDLQCLEDELGPLRHVLDLTQSKSFQLEDAENFISNIRVTVVKLKGSDNTFECQFDDESATVVDFLRRWIAFCQSIISTSPQ

SEQ ID NO: 36 - protein, 349 aa, synthetic construct
  REGION 1..349: note = Synthetic peptide
CSQPLDVILLLDGSSSFPASYFDEMKSFAKAFISKANIGPRLTQVSVLQYGSITTIDVPWNVVPEKAHLLSLVDVMQREGGPSQIGDALGFAVRYLTSEMHGARPGASKAVVILVTDVSVDSVDAAADAARSNRVTVFPIGIGDRYDAAQLRILAGPAGDSNVVKLQRIEDLPTMVTLGNSFLHKLCSGFVRIGGGSGGGSPTSSSTSSSTAEAQQQQQQQQQQQQHLEQLLMDLQELLSRMENYRNLKLPRMLTFKFYLPKQATELKDLQCLEDELGPLRHVLDLTQSKSFQLEDAENFISNIRVTVVKLKGSDNTFECQFDDESATVVDFLRRWIAFCQSIISTSPQ

SEQ ID NO: 37 - protein, 153 aa, Homo sapiens
MYRMQLLSCIALSLALVTNSAPTSSSTKKTQLQLEHLLLDLQMILNGINNYKNPKLTRMLTFKFYMPKKATELKHLQCLEEELKPLEEVLNLAQSKNFHLRPRDLISNINVIVLELKGSETTFMCEYADETATIVEFLNRWITFCQSIISTLT

SEQ ID NO: 38 - protein, 346 aa, synthetic construct
  REGION 1..346: note = synthetic peptide
CSQPLDVILLLDGSSSFPASYFDEMKSFAKAFISKANIGPRLTQVSVLQYGSITTIDVPWNVVPEKAHLLSLVDVMQREGGPSQIGDALGFAVRYLTSEMHGARPGASKAVVILVTDVSVDSVDAAADAARSNRVTVFPIGIGDRYDAAQLRILAGPAGDSNVVKLQRIEDLPTMVTLGNSFLHKLCSGFVRIMYRMQLLSCIALSLALVTNSAPTSSSTKKTQLQLEHLLLDLQMILNGINNYKNPKLTRMLTFKFYMPKKATELKHLQCLEEELKPLEEVLNLAQSKNFHLRPRDLISNINVIVLELKGSETTFMCEYADETATIVEFLNRWITFCQSIISTLT

SEQ ID NO: 39 - protein, 182 aa, Mus musculus
MNNRWILHAAFLLCFSTTALSINYKQLQLQERTNIRKCQELLEQLNGKINLTYRADFKIPMEMTEKMQKSYTAFAIQEMLQNVFLVFRNNFSSTGWNETIVVRLLDELHQQTVFLKTVLEEKQEERLTWEMSSTALHLKSYYWRVQRYLKLMKYNSYAWMVVRAEIFRNFLIIRRLTRNFQN

SEQ ID NO: 40 - protein, 166 aa, Homo sapiens
MSYNLLGFLQRSSNFQCQKLLWQLNGRLEYCLKDRMNFDIPEEIKQLQQFQKEDAALTIYEMLQNIFAIFRQDSSSTGWNETIVENLLANVYHQINHLKTVLEEKLEKEDFTRGKLMSSLHLKRYYGRILHYLKAKEYSHCAWTIVRVEILRNFYFINRLTGYLRN

SEQ ID NO: 41 - protein, 167 aa, Mus musculus
CDLPHTYNLRNKRALKVLAQMRRLPFLSCLKDRQDFGFPLEKVDNQQIQKAQAIPVLRDLTQQTLNLFTSKASSAAWNTTLLDSFCNDLHQQLNDLQTCLMQQVGVQEPPLTQEDALLAVRKYFHRITVYLREKKHSPCAWEVVRAEVWRALSSSVNLLPRLSEEKE

SEQ ID NO: 42 - protein, 72 aa, Homo sapiens
MALTFALLVALLVLSCKSSCSVGCDLPQTHSLGSRRTLMLLAQMRKISLFSCLKDRHDFGFPQEEFGNQFQK

SEQ ID NO: 43 - protein, 93 aa, Mus musculus
VGTEVLEESSCVNLQTQRLPVQKIKTYIIWEGAMRAVIFVTKRGLKICADPEAKWVKAAIKTVDGRASTRKNMAETVPTGAQRSTSTAITLTG

SEQ ID NO: 44 - protein, 92 aa, Homo sapiens
GSEVSDKRTCVSLTTQRLPVSRIKTYTITEGSLRAVIFITKRGLKVCADPQATWVRDVVRSMDRKSNTRNNMIQTKPTGTQQSTNTAVTLTG

SEQ ID NO: 45 - protein, 234 aa, Mus musculus
GTTCPPPVSIEHADIRVKNYSVNSRERYVCNSGFKRKAGTSTLIECVINKNTNVAHWTTPSLKCIRDPSLAHYSPVPTVVTPKVTSQPESPSPSAKEPEASGGSGGGGSGGGSGGGGSLQNWIDVRYDLEKIESLIQSIHIDTTLYTDSDFHPSCKVTAMNCFLLELQVILHEYSNMTLNETVRNVLYLANSTLSSNKNVAESGCKECEELEEKTFTEFLQSFIRIVQMFINTS

SEQ ID NO: 46 - protein, 424 aa, Homo sapiens
ITCPPPMSVEHADIWVKSYSLYSRERYICNSGFKRKAGTSSLTECVLNKATNVAHWTTPSLKCIRDVDDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGKGGGGSGGGGSGGGGSNWVNVISDLKKIEDLIQSMHIDATLYTESDVHPSCKVTAMKCFLLELQVISLESGDASIHDTVENLIILANDSLSSNGNVTESGCKECEELEEKNIKEFLQSFVHIVQMFINTS

SEQ ID NO: 47 - protein, 278 aa, synthetic construct
  REGION 1..278: note = Synthetic polypeptide
CSQPLDVILLLDGSSSFPASYFDEMKSFAKAFISKANIGPRLTQVSVLQYGSITTIDVPWNVVPEKAHLLSLVDVMQREGGPSQIGDALGFAVRYLTSEMHGARPGASKAVVILVTDVSVDSVDAAADAARSNRVTVFPIGIGDRYDAAQLRILAGPAGDSNVVKLQRIEDLPTMVTLGNSFLHKLCSGFVRIGGGGSGGGGSAPMGSDPPTSCCFSYTSRQLHRSFVMDYYETSSLCSKPAVVFLTKRGRQICANPSEPWVTEYMSDLELNHHHHHH

(19) United States
(12) Patent Application Publication
(10) Pub. No.: US 2026/0049115 A1
(43) Pub. Date: Feb. 19, 2026

(54) METHODS AND COMPOSITIONS FOR TREATING CANCER WITH ECM-AFFINITY PEPTIDES LINKED TO CYTOKINES

(71) Applicant: The University of Chicago, Chicago, IL (US)

(72) Inventors: Jeffrey A. HUBBELL, Chicago, IL (US); Jun ISHIHARA, Chicago, IL (US); Ako ISHIHARA, Chicago, IL (US); Koichi SASAKI, Chicago, IL (US); Melody SWARTZ, Chicago, IL (US); Aslan MANSUROV, Chicago, IL (US)

(73) Assignee: The University of Chicago, Chicago, IL (US)

(21) Appl. No.: 19/249,789

(22) Filed: Jun. 25, 2025

Related U.S. Application Data
(63) Continuation of application No. 15/733,582, now Pat. No. 12,365,714.
(60) Provisional application No. 62/638,520, filed on Mar. 5, 2018.
(60) Provisional application No. 62/727,156, filed on Sep. 5, 2018.

(51) Int. Cl. C07K 14/55; A61K 9/00; A61K 38/20; A61K 45/06; A61P 35/00; C07K 14/755

(52) U.S. Cl. CPC C07K 14/55; A61K 9/0019; A61K 38/2013; A61K 45/06; A61P 35/00; C07K 14/755; C07K 2319/00

(57) ABSTRACT

The methods and compositions described herein address the need in the art by providing compositions and methods for a therapy with a cytokine that is specifically targeted to and/or retained intra-or peri-tumorally, limiting systemic exposure and reducing side-effects. Accordingly, aspects of the disclosure relate to a composition comprising an immunotherapeutic antibody operatively linked to an extracellular matrix (ECM)-affinity peptide. An ECM-affinity peptide is one that has affinity for an extracellular matrix protein.

### CROSS-REFERENCE TO RELATED APPLICATIONS

This application is a continuation of U.S. patent application Ser. No. 15/733,582, filed Sep. 3, 2020, which is a national phase application under 35 U.S.C. § 371 of International Application No. PCT/US2019/020685, filed Mar. 5, 2019, which claims the benefit of priority to U.S. Provisional Patent Application Nos. 62/638,520 filed Mar. 5, 2018 and 62/727,156 filed Sep. 5, 2018, all of which are incorporated herein by reference in their entireties.

### SEQUENCE LISTING

The instant application contains a Sequence Listing which has been submitted in XML format and is hereby incorporated by reference in its entirety. Said XML copy, created on Jun. 18, 2025, is named ARCDP0652USC1-Sequence-Listing.xml and is 51,558 bytes in size.

### BACKGROUND

### I. Field of the Invention

The invention generally relates to the field of medicine. More particularly, it concerns compositions and methods involving nucleotide constructs and proteins-including engineered cytokines for targeting areas associated with vascular permeability, such as tumors.

### II. Background

The immune system plays a critical role in the pathogenesis of a wide variety of cancers. When cancers progress, it is widely believed that the immune system either fails to respond sufficiently or fails to respond appropriately, allowing cancer cells to grow. Currently, standard medical treatments for cancer including chemotherapy, surgery, radiation therapy and cellular therapy have clear limitations with regard to both efficacy and toxicity. To date, these approaches have met with varying degrees of success dependent upon the type of cancer, general health of the patient, stage of disease at the time of diagnosis, etc. Improved strategies that combine specific manipulation of the immune response to cancer in combination with standard medical treatments may provide a means for enhanced efficacy and decreased toxicity of cancer therapies.

Numerous cytokines have been shown to play a role in regulation of the immune response to tumors. However, direct administration of cytokines for cancer therapy may not be practical, as they are often toxic when administered systemically. (See, for example, Asher et al., J. Immunol. 146: 3227-3234, 1991; Havell et al., J. Exp. Med. 167: 1067-1085, 1988.) There remains a need for additional compositions and methods to provide more effective and less toxic cytokine therapies for cancer.

### SUMMARY OF INVENTION

The methods and compositions described herein provide needed compositions and methods for targeting or localizing a therapy in areas having vascular leak or permeability by providing a cytokine that is specifically targeted to and/or retained by collagen limiting systemic exposure and reducing side-effects associated with the cytokine. Certain embodiments are directed to the administration of a cancer therapy that localizes in a tumor due to vascular permeability of the tumor. Accordingly, aspects of the disclosure relate to a composition comprising a cytokine operatively linked to an extracellular matrix (ECM)-affinity peptide, e.g., a collagen binding domain (CBD). An ECM-affinity peptide is one that has affinity for and binds to an extracellular matrix protein, such as collagen.

In one embodiment, the ECM-affinity peptide comprises a peptide from von Willebrand Factor (vWF) or decorin. In one embodiment, the ECM-affinity peptide comprises, consist essentially of, or consist of at least, at most, or about 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 60, 70, 80, 90, 100, 110, 120, 130, 140, 150, 160, 170, 180, 190, 200, 210, 220, 230, 240, 250, 300, 400, 500, 600, 700, 800, 900, 1000, 1500, 2000, to 2800, including all values and ranges there between, contiguous amino acids of vWF A3 domain (SEQ ID NO:3); vWF A1 domain (SEQ ID NO:11); vWF (SEQ ID NO:13); decorin (SEQ ID NO:15), or vWF A3 domain (SEQ ID NO:34). In certain aspects an ECM-affinity peptide can comprise amino acids 1 to 193 of vWF A3 domain (SEQ ID NO:34).

ECM-affinity peptides can include a peptide or protein fragment having at least, or about 70, 75, 80, 85, 90, 91, 92, 93, 94, 95, 96, 97, 98, 99, 100% identity to vWF A3 domain (SEQ ID NO:3); vWF A1 domain (SEQ ID NO:11); vWF (SEQ ID NO:13); decorin (SEQ ID NO:15), or vWF A3 domain (SEQ ID NO:34), fragments including: (A) a fragment of SEQ ID NO:3 includes a fragment of segment starting at amino acid 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, or 200 and ending at amino acid 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 201, 202, 203, 204, or 205; (B) a fragment of SEQ ID NO:11 includes a fragment of segment starting at amino acid 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 201, 202, 203, 204, 205, 206, 207, 208, 209, 210, 211, or 212 and ending at amino acid 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 201, 202, 203, 204, 205, 206, 207, 208, 209, 210, 211, 212, 213, 214, 215, 216, 217, 218, 219, 220, 221, or 222; (C) a fragment of SEQ ID NO:13 a fragment of segment starting at amino acid 1, 10, 20, 30, 40, 50, 60, 70, 80, 90, 100, 110, 120, 130, 140, 150, 160, 170, 180, 190, 200, 210, 220, 230, 240, 250, 260, 270, 280, 290, 300, 310, 320, 330, 340, 350, 360, 370, 380, 390, 400, 410, 420, 430, 440, 450, 460, 470, 480, 490, 500, 510, 520, 530, 540, 550, 560, 570, 580, 590, 600, 610, 620, 630, 640, 650, 660, 670, 680, 690, 700, 710, 720, 730, 740, 750, 760, 770, 780, 790, 800, 810, 820, 830, 840, 850, 860, 870, 880, 890, 900, 910, 920, 930, 940, 950, 960, 970, 980, 990, 1000, 1010, 1020, 1030, 1040, 1050, 1060, 1070, 1080, 1090, 1100, 1110, 1120, 1130, 1140, 1150, 1160, 1170, 1180, 1190, 1200, 1210, 1220, 1230, 1240, 1250, 1260, 1270, 1280, 1290, 1300, 1310, 1320, 1330, 1340, 1350, 1360, 1370, 1380, 1390, 1400, 1410, 1420, 1430, 1440, 1450, 1460, 1470, 1480, 1490, 1500, 1510, 1520, 1530, 1540, 1550, 1560, 1570, 1580, 1590, 1600, 1610, 1620, 1630, 1640, 1650, 1660, 1670, 1680, 1690, 1700, 1710, 1720, 1730, 1740, 1750, 1760, 1770, 1780, 1790, 1800, 1810, 1820, 1830, 1840, 1850, 1860, 1870, 1880, 1890, 1900, 1910, 1920, 1930, 1940, 1950, 1960, 1970, 1980, 1990, 2000, 2010, 2020, 2030, 2040, 2050, 2060, 2070, 2080, 2090, 2100, 2110, 2120, 2130, 2140, 2150, 2160, 2170, 2180, 2190, 2200, 2210, 2220, 2230, 2240, 2250, 2260, 2270, 2280, 2290, 2300, 2310, 2320, 2330, 2340, 2350, 2360, 2370, 2380, 2390, 2400, 2410, 2420, 2430, 2440, 2450, 2460, 2470, 2480, 2490, 2500, 2510, 2520, 2530, 2540, 2550, 2560, 2570, 2580, 2590, 2600, 2610, 2620, 2630, 2640, 2650, 2660, 2670, 2680, 2690, 2700, 2710, 2720, 2730, 2740, 2750, 2760, 2770, 2780, 2790, or 2800, including all values and ranges there between, and ending at amino acid 10, 20, 30, 40, 50, 60, 70, 80, 90, 100, 110, 120, 130, 140, 150, 160, 170, 180, 190, 200, 210, 220, 230, 240, 250, 260, 270, 280, 290, 300, 310, 320, 330, 340, 350, 360, 370, 380, 390, 400, 410, 420, 430, 440, 450, 460, 470, 480, 490, 500, 510, 520, 530, 540, 550, 560, 570, 580, 590, 600, 610, 620, 630, 640, 650, 660, 670, 680, 690, 700, 710, 720, 730, 740, 750, 760, 770, 780, 790, 800, 810, 820, 830, 840, 850, 860, 870, 880, 890, 900, 910, 920, 930, 940, 950, 960, 970, 980, 990, 1000, 1010, 1020, 1030, 1040, 1050, 1060, 1070, 1080, 1090, 1100, 1110, 1120, 1130, 1140, 1150, 1160, 1170, 1180, 1190, 1200, 1210, 1220, 1230, 1240, 1250, 1260, 1270, 1280, 1290, 1300, 1310, 1320, 1330, 1340, 1350, 1360, 1370, 1380, 1390, 1400, 1410, 1420, 1430, 1440, 1450, 1460, 1470, 1480, 1490, 1500, 1510, 1520, 1530, 1540, 1550, 1560, 1570, 1580, 1590, 1600, 1610, 1620, 1630, 1640, 1650, 1660, 1670, 1680, 1690, 1700, 1710, 1720, 1730, 1740, 1750, 1760, 1770, 1780, 1790, 1800, 1810, 1820, 1830, 1840, 1850, 1860, 1870, 1880, 1890, 1900, 1910, 1920, 1930, 1940, 1950, 1960, 1970, 1980, 1990, 2000, 2010, 2020, 2030, 2040, 2050, 2060, 2070, 2080, 2090, 2100, 2110, 2120, 2130, 2140, 2150, 2160, 2170, 2180, 2190, 2200, 2210, 2220, 2230, 2240, 2250, 2260, 2270, 2280, 2290, 2300, 2310, 2320, 2330, 2340, 2350, 2360, 2370, 2380, 2390, 2400, 2410, 2420, 2430, 2440, 2450, 2460, 2470, 2480, 2490, 2500, 2510, 2520, 2530, 2540, 2550, 2560, 2570, 2580, 2590, 2600, 2610, 2620, 2630, 2640, 2650, 2660, 2670, 2680, 2690, 2700, 2710, 2720, 2730, 2740, 2750, 2760, 2770, 2780, 2790, 2800 281 or 2813, including all values and ranges there between; (D) a fragment of SEQ ID NO:15 starting at amino acid 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 201, 202, 203, 204, 205, 206, 207, 208, 209, 210, 211, 212, 213, 214, 215, 216, 217, 218, 219, 220, 221, 222, 223, 224, 225, 226, 227, 228, 229, 230, 231, 232, 233, 234, 235, 236, 237, 238, 239, 240, 241, 242, 243, 244, 245, 246, 247, 248, 249, 250, 251, 252, 253, 254, 255, 256, 257, 258, 259, 260, 261, 262, 263, 264, 265, 266, 267, 268, 269, 270, 271, 272, 273, 274, 275, 276, 277, 278, 279, 280, 281, 282, 283, 284, 285, 286, 287, 288, 289, 290, 291, 292, 293, 294, 295, 296, 297, 298, 299, 300, 301, 302, 303, 304, 305, 306, 307, 308, 309, 310, 311, 312, 313, 314, 315, 316, 317, 318, 319, 320, 321, 322, 323, 324, 325, 326, 327, 328, 329, 330, 331, 332, or 333 and ending at amino acid 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 201, 202, 203, 204, 205, 206, 207, 208, 209, 210, 211, 212, 213, 214, 215, 216, 217, 218, 219, 220, 221, 222, 223, 224, 225, 226, 227, 228, 229, 230, 231, 232, 233, 234, 235, 236, 237, 238, 239, 240, 241, 242, 243, 244, 245, 246, 247, 248, 249, 250, 251, 252, 253, 254, 255, 256, 257, 258, 259, 260, 261, 262, 263, 264, 265, 266, 267, 268, 269, 270, 271, 272, 273, 274, 275, 276, 277, 278, 279, 280, 281, 282, 283, 284, 285, 286, 287, 288, 289, 290, 291, 292, 293, 294, 295, 296, 297, 298, 299, 300, 301, 302, 303, 304, 305, 306, 307, 308, 309, 310, 311, 312, 313, 314, 315, 316, 317, 318, 319, 320, 321, 322, 323, 324, 325, 326, 327, 328, 329, 330, 331, 332, 333, 334, 335, 336, 337, 338, 339, 340, 341, 342, or 343; (E) a fragment of SEQ ID NO:34 starting at amino acid 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, or 186 and ending at amino acid 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, or 196.

In certain aspect any polypetide, peptide, or fragement thereof need not contain all amino acids of the referenced sequence identifier and may have 1, 2, 3, 4, 5, 6, 7, 8, 9, 10, 11, 12, 13, 14, 15, 16, 17, 18, 19, 20, 30, 40, 50 or so amino acids removed or deleted while maintaining at least one activity of the polypeptide or peptide, e.g., ECM or collagen binding. In further aspects any polypetide or peptide may contain additional amino acids at the terminus or inserted internally in the amino acid sequence of the referenced sequence identifier and may have 1, 2, 3, 4, 5, 6, 7, 8, 9, 10, 11, 12, 13, 14, 15, 16, 17, 18, 19, 20, 30, 40, 50 or so amino acids fused to the amino terminus, carboxy terminus, inserted in the amino acid sequence or a combination thereof while maintaining at least one activity of the polypeptide or peptide e.g., ECM or collagen binding. It is specifically contemplated that any of the ECM-affinity peptides disclosed herein may be excluded from some embodiments (e.g., P1GF-2 and CXCL-12γ peptides). In one embodiment, the ECM-affinity peptide comprises a peptide that has at least or at most 70, 72, 74, 76, 78, 80, 82, 84, 86, 88, 90, 92, 94, 96, 98, 99, or 100% identity (or any range derivable therein) to one of SEQ ID NO: 3; SEQ ID NO: 11; SEQ ID NO:13; SEQ ID NO:15, or SEQ ID NO:34 or to a peptide segment or fragment from SEQ ID NO:3; SEQ ID NO:11; SEQ ID NO:13; SEQ ID NO:15, or SEQ ID NO: 34. A peptide segment or fragment can include 10, 20, 30, 40, 50, 60, 70, 80, 90, 100, 110, 120, 130, 140, 150, 160, 170, 180, 190, 200, 210, 220, or more consecutive amino acids of SEQ ID NO:3; SEQ ID NO:11; SEQ ID NO:13; SEQ ID NO:15, or SEQ ID NO:34. In some embodiments, the peptide is at least 85% identical to SEQ ID NO:3; SEQ ID NO:11; SEQ ID NO:13; SEQ ID NO:15, or SEQ ID NO:34. In some embodiments, the peptide comprises or consists of or consist essentially of SEQ ID NO:3; SEQ ID NO:11; SEQ ID NO:13; SEQ ID NO:15, or SEQ ID NO:34 or a fragment thereof. In one embodiment, the ECM-affinity peptide comprises a von Willebrand factor (vWF) peptide. In some embodiments, the VWF peptide is a vWF A1 or A3 peptide. In some embodiments, the VWF peptide comprises a peptide that is at least 85% identical to all or part of SEQ ID NO:3, SEQ ID NO:11, or SEQ ID NO:34. In some embodiments, the VWF peptide comprises SEQ ID NO:3 or SEQ ID NO:11 or SEQ ID NO:34 or is a fragment thereof.

In some embodiments, the ECM-affinity peptide comprises a decorin peptide. In some embodiments, the peptide is at least 85% identical to all or part of SEQ ID NO: 15. comprises 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 75, 100, 125, 15, 175, 200, 225, 250, 275, 300, 325, to 343 contiguous amino acids (including all values and ranges there between) of SEQ ID NO:15. In one embodiment, the ECM-affinity peptide comprises a peptide that has at least or at most 70, 72, 74, 76, 78, 80, 82, 84, 86, 88, 90, 92, 94, 96, 98, 99, or 100% identity (or any range derivable therein) to SEQ ID NO:15 or to a peptide segment of SEQ ID NO:15. In some embodiments, the peptide is at least 85% identical to SEQ ID NO:15. In some embodiments, the peptide comprises or consists of or consist essentially of SEQ ID NO:15 or a fragment thereof.

In some embodiments, the peptide is covalently linked to the cytokine. In certain asepcts the peptide is an amino terminal or carboxy terminal fusion with a cytokine polypeptide. In some embodiments, the peptide is crosslinked to the cytokine through a bifunctional crosslinker.

In other embodiments the cytokine peptide can be selected from hIL-2 (SEQ ID NO:37); mIL-2 (SEQ ID NO:37); hIL-15 (SEQ ID NO:16), mIL-15 (SEQ ID NO:17), hIL-21 (SEQ ID NO:18), mIL-21 (SEQ ID NO:19), hIL-12 p35 (SEQ ID NO:20), hIL-12 p40 (SEQ ID NO:21), mIL-12 p35 (SEQ ID NO:22), mIL-12 p35 (SEQ ID NO:23), hCCL4 (SEQ ID NO:24), mCCL4 (SEQ ID NO:25), hCCL21 (SEQ ID NO:26), mCCL21 (SEQ ID NO:27), hCXCL9 (SEQ ID NO:28), mCXCL9 (SEQ ID NO:29), hCXCL10 (SEQ ID NO:30), mCXCL10 (SEQ ID NO:31), hVEGF-C (SEQ ID NO:32), mVEGF-C (SEQ ID NO:33), mIFNβ (SEQ ID NO:39), hIFNβ (SEQ ID NO:40), mIFNα2 (SEQ ID NO:41), hIFNα2 (SEQ ID NO:42), mXCL1 (SEQ ID NO:43), hXCL1 (SEQ ID NO:44), mIL-15 super agonist (SEQ ID NO:45), or hIL-15 super agonist (SEQ ID NO:46). The cytokine portion of the ECM-affinity peptide cytokine conjugate can comprise 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 55, 60, 65, 70, 75, 80, 85, 90, 95, 100, 105, 110, 115, 120, 125, 130, 135, 140, 145, 150 contiguous amino acids of hIL-15 (SEQ ID NO:16), mIL-15 (SEQ ID NO:17), hIL-21 (SEQ ID NO:18), mIL-21 (SEQ ID NO:19), hIL-12 p35 (SEQ ID NO:20), hIL-12 p40 (SEQ ID NO:21), mIL-12 p35 (SEQ ID NO:22), mIL-12 p35 (SEQ ID NO:23), hCCL4 (SEQ ID NO:24), mCCL4 (SEQ ID NO:25), hCCL21 (SEQ ID NO:26), mCCL21 (SEQ ID NO:27), hCXCL9 (SEQ ID NO:28), mCXCL9 (SEQ ID NO:29), hCXCL10 (SEQ ID NO:30), mCXCL10 (SEQ ID NO:31), hVEGF-C (SEQ ID NO:32), mVEGF-C (SEQ ID NO:33), mIFNβ (SEQ ID NO:39), hIFNβ (SEQ ID NO:40), mIFNα2 (SEQ ID NO:41), hIFNα2 (SEQ ID NO:42), mXCL1 (SEQ ID NO:43), hXCL1 (SEQ ID NO:44), mIL-15 super agonist (SEQ ID NO:45), or hIL-15 super agonist (SEQ ID NO:46). In one embodiment, the cytokinie peptide comprises a peptide that has at least or at most 70, 72, 74, 76, 78, 80, 82, 84, 86, 88, 90, 92, 94, 96, 98, 99, or 100% identity (or any range derivable therein) to hIL-15 (SEQ ID NO:16), mIL-15 (SEQ ID NO:17), hIL-21 (SEQ ID NO:18), mIL-21 (SEQ ID NO:19), hIL-12 p35 (SEQ ID NO:20), hIL-12 p40 (SEQ ID NO:21), mIL-12 p35 (SEQ ID NO:22), mIL-12 p35 (SEQ ID NO:23), hCCL4 (SEQ ID NO:24), mCCL4 (SEQ ID NO:25), hCCL21 (SEQ ID NO:26), mCCL21 (SEQ ID NO:27), hCXCL9 (SEQ ID NO:28), mCXCL9 (SEQ ID NO:29), hCXCL10 (SEQ ID NO:30), mCXCL10 (SEQ ID NO:31), hVEGF-C (SEQ ID NO:32), m VEGF-C (SEQ ID NO:33), mIFNβ (SEQ ID NO:39), hIFNβ (SEQ ID NO:40), mIFNα2 (SEQ ID NO:41), hIFNα2 (SEQ ID NO:42), mXCL1 (SEQ ID NO:43), hXCL1 (SEQ ID NO:44), mIL-15 super agonist (SEQ ID NO: 45), or hIL-15 super agonist (SEQ ID NO:46) or to a peptide segment of hIL-15 (SEQ ID NO:16), mIL-15 (SEQ ID NO:17), hIL-21 (SEQ ID NO:18), mIL-21 (SEQ ID NO:19), hIL-12 p35 (SEQ ID NO:20), hIL-12 p40 (SEQ ID NO:21), mIL-12 p35 (SEQ ID NO:22), mIL-12 p35 (SEQ ID NO:23), hCCL4 (SEQ ID NO:24), mCCL4 (SEQ ID NO:25), hCCL21 (SEQ ID NO:26), mCCL21 (SEQ ID NO:27), hCXCL9 (SEQ ID NO:28), mCXCL9 (SEQ ID NO:29), hCXCL10 (SEQ ID NO:30), mCXCL10 (SEQ ID NO:31), hVEGF-C (SEQ ID NO:32), m VEGF-C (SEQ ID NO:33), mIFNβ (SEQ ID NO:39), hIFNβ (SEQ ID NO:40), mIFNα2 (SEQ ID NO:41), hIFNα2 (SEQ ID NO:42), mXCL1 (SEQ ID NO:43), hXCL1 (SEQ ID NO:44), mIL-15 super agonist (SEQ ID NO:45), or hIL-15 super agonist (SEQ ID NO:46) that retains cytokine activity. In some embodiments, the peptide is at least 85% identical to hIL-15 (SEQ ID NO:16), mIL-15 (SEQ ID NO:17), hIL-21 (SEQ ID NO:18), mIL-21 (SEQ ID NO:19), hIL-12 p35 (SEQ ID NO:20), hIL-12 p40 (SEQ ID NO:21), mIL-12 p35 (SEQ ID NO:22), mIL-12 p35 (SEQ ID NO:23), hCCL4 (SEQ ID NO:24), mCCL4 (SEQ ID NO:25), hCCL21 (SEQ ID NO:26), mCCL21 (SEQ ID NO:27), hCXCL9 (SEQ ID NO:28), mCXCL9 (SEQ ID NO:29), hCXCL10 (SEQ ID NO:30), mCXCL10 (SEQ ID NO:31), hVEGF-C (SEQ ID NO:32), mVEGF-C (SEQ ID NO:33), mIFNβ (SEQ ID NO:39), hIFNβ (SEQ ID NO:40), mIFNα2 (SEQ ID NO:41), hIFNα2 (SEQ ID NO:42), mXCL1 (SEQ ID NO:43), hXCL1 (SEQ ID NO:44), mIL-15 super agonist (SEQ ID NO:45), or hIL-15 super agonist (SEQ ID NO:46). In some embodiments, the peptide comprises or consists of or consist essentially of hIL-15 (SEQ ID NO:16), mIL-15 (SEQ ID NO: 17), hIL-21 (SEQ ID NO:18), mIL-21 (SEQ ID NO:19), hIL-12 p35 (SEQ ID NO:20), hIL-12 p40 (SEQ ID NO:21), mIL-12 p35 (SEQ ID NO:22), mIL-12 p35 (SEQ ID NO:23), hCCL4 (SEQ ID NO:24), mCCL4 (SEQ ID NO:25), hCCL21 (SEQ ID NO:26), mCCL21 (SEQ ID NO:27), hCXCL9 (SEQ ID NO:28), mCXCL9 (SEQ ID NO:29), hCXCL10 (SEQ ID NO:30), mCXCL10 (SEQ ID NO:31), hVEGF-C (SEQ ID NO:32), m VEGF-C (SEQ ID NO:33), mIFNβ (SEQ ID NO:39), hIFNβ (SEQ ID NO:40), mIFNα2 (SEQ ID NO:41), hIFNα2 (SEQ ID NO:42), mXCL1 (SEQ ID NO:43), hXCL1 (SEQ ID NO:44), mIL-15 super agonist (SEQ ID NO:45), or hIL-15 super agonist (SEQ ID NO:46), or a fragment thereof.

In certain embodiments the IL-2 portion of the ECM-affinity peptide cytokine conjugate comprises 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 55, 60, 65, 70, 75, 80, 85, 90, 95, 100, 105, 110, 115, 120, 125, 130, 135, 140, 145, 150 contiguous amino acids of SEQ ID NO:35 or SEQ ID NO:37. In one embodiment, the IL-2 peptide comprises a peptide that has at least or at most 70, 72, 74, 76, 78, 80, 82, 84, 86, 88, 90, 92, 94, 96, 98, 99, or 100% identity (or any range derivable therein) to SEQ ID NO:35 or SEQ ID NO:37 or to a peptide segment SEQ ID NO:35 or SEQ ID NO:37 that retains an IL-2 activity. In some embodiments, the peptide is at least 85% identical to SEQ ID NO:35 or SEQ ID NO:37. In some embodiments, the peptide comprises or consists of or consist essentially of SEQ ID NO:35 or SEQ ID NO:37 or a fragment thereof. His-tagged mouse IL-2 having the following sequence—PTSSSTSSSTAEAQQQQQQQQQQQQHLEQLLMDLQELLSRMENYRNLKLPRMLT FKFYLPKQATELKDLQCLEDELGPLRHVLDLTQSKSFQLEDAENFISNIRVTVVKLKGSD NTFECQFDDESATVVDFLRRWIAFCQSIISTSPQHHHHHH (SEQ ID NO:35). Human IL-2 having the following sequence—MYRMQLLSCIALSLALVTNSAPTSSSTKKTQLQLEH LLLDLQMILNGINNYKNPKLTRMLTFKFYMPKKATELKHLQCLEEELKPLEEVLNLAQS KNFHLRPRDLISNINVIVLELKGSETTFMCEYADETATIVEFLNRWITFCQSIISTLT (SEQ ID NO: 37).

Linkers, such as amino acid or peptidimimetic sequences may be inserted between the peptide and cytokine sequence. Linkers may have one or more properties that include a flexible conformation, an inability to form an ordered secondary structure or a hydrophobic or charged character which could promote or interact with either domain. Examples of amino acids typically found in flexible protein regions may include Gly, Asn, and Ser. Other near neutral amino acids, such as Thr and Ala, may also be used in the linker sequence. The length of the linker sequence may vary without significantly affecting the function or activity of the fusion protein (see, U.S. Pat. No. 6,087,329). In a particular aspect, a peptide and cytokine are joined by a peptide sequence having from about 1, 2, 3, 4, 5, 6, 7, 8, 9, 10, 11, 12, 13, 14, 15, 16, 17, 18, 19, 20, 21, 22, 23, 24, to 25 amino acid residues. Examples of linkers may also include chemical moieties and conjugating agents, such as sulfo-succinimidyl derivatives (sulfo-SMCC, sulfo-SMPB), disuccinimidyl suberate (DSS), disuccinimidyl glutarate (DSG) and disuccinimidyl tartrate (DST). Linkers further include a linear carbon chain, such as C_{N} (where N=1-100 carbon atoms, e.g., C₁, C₂, C₃, C₄, C₅, C₆, C₇, C₈). In some embodiments, the linker can be a dipeptide linker, such as a valine-citrulline (val-cit), a phenylalanine-lysine (phe-lys) linker, or maleimidocapronic-valine-citruline-p-aminobenzyloxycarbonyl (vc) linker. In some embodiments, the linker is sulfosuccinimidyl-4-[N-maleimidomethyl] cyclohexane-1-carboxylate (smcc). Sulfo-smcc conjugation occurs via a maleimide group which reacts with sulfhydryls (thiols, —SH), while its Sulfo-NHS ester is reactive toward primary amines (as found in Lysine and the protein or peptide N-terminus). Further, the linker may be maleimidocaproyl (mc).

In some embodiments, the ratio of peptide to cytokine is about 1:1 to 10:1. In some embodiments, the ratio of peptide to cytokine is at least, at most, or about 1:1, 2:1, 3:1, 4:1, 5:1, 6:1, 7:1, 8:1, 9:1, 10:1, 11:1, 12:1, 13:1, 14:1, 15:1, 16:1, 17:1, 18:1, 19:1, 20:1, 21:1, 22:1, 23:1, 24:1, 25:1, 30:1, 35:1, 40:1, 45:1, 50:1, or 100:1 (or any range there between).

Further aspects relate to methods for treating cancer in a subject comprising administering a composition including a ECM-affinity peptide coupled to a cytokine to a subject. In some embodiments, the composition is administered intravenously, or by intra-tumoral, peri-tumoral, intraarterial, or transcatheter injection. In some embodiments, the vWF or decorin containing polypeptides described herein (i.e., those having one or more peptides having all or part of a sequence of SEQ ID NO:3, SEQ ID NO:11, SEQ ID NO: 13, SEQ ID NO: 15, SEQ ID NO:34) are administered systemically. The systemic administration may be parenteral or intravenous, for example.

In some embodiments, the administered dose of a cytokine operatively linked to the ECM-affinity peptide is less than the minimum effective dose of the cytokine administered without the ECM-affinity peptide. In some embodiments, the administered dose of the cytokine operatively linked to the ECM-affinity peptide is at least 10% less than the minimum effective dose of the cytokine administered without the ECM-affinity peptide. In some embodiments, the administered dose of the cytokine operatively linked to the ECM-affinity peptide is at least 5, 10, 15, 20, 25, 30, 35, 40, 45, 50, 55, 60, 65, 70, 75, or 80% less (or any derivable range therein) than the minimum effective dose of the cytokine administered without the ECM-affinity peptide.

In some embodiments, the subject has been diagnosed with a cancer. In some embodiments, the cancer is melanoma, colon cancer, lung cancer, prostate cancer, ovarian cancer, testicular cancer, brain cancer, glioblastoma, pediatric tumors, germ cell tumors, rectal cancer, gastric cancer, esophageal cancer, tracheal cancer, head and neck cancer, pancreatic cancer, liver cancer, breast cancer, cervical cancer, and vulvar cancer. In certain embodiments, the cancer is melanoma or colon cancer. In some embodiments, the cancer is non-hematological. In some embodiments, the cancer includes a solid tumor. In some embodiments, the cancer is distant metastasis. In some embodiments, the patient has been previously treated for the cancer. In some embodiments, the subject was resistant to the previous cancer treatment. In some embodiments, the subject was determined to be a poor responder to the cancer treatment.

The methods can further include administering an additional cancer therapy. In some embodiments, the additional cancer therapy comprises radiation, vaccination, chemotherapy, adoptive T-cell therapy, cytokine therapy, anti-CD47 antibodies, anti-GD2 antibodies, or immunologic adjuvants.

In some embodiments, the method further comprises administration of a second cytokine operatively linked to the same or differenct ECM-affinity peptide.

The term “cytokine polypeptide” as used herein refers to a polypeptide, which is cytokine or a receptor binding domain thereof and retains at a porition of cytokine activity.

The term “cytokine activity” as used herein refers to the activities which cytokines possess or are able to exert in vivo, including but not limited to the promotion of proliferation, immunoglobulin class switching and antibody secretion of B cells; differentiation of memory B cells, or prevention of their apoptosis; promoting macrophages' secretion of interleukin-12 to activate type I helper T cells or secrete chemokines; promoting macrophages to produce nitric oxide to enhance the defense capability against microorganisms; promoting the maturation and activation of dendritic cells; regulation of the maturation and differentiation of T cells; promoting the cytotoxicity and the production of a variety of different cytokines of natural killer cells; activation of monocytes and macrophages; and stimulation of T cells and B cells to continuously express MHC, etc.

The term “chemokine polypeptide” as used herein refers to a cytokine polypeptide that is a chemokine or a receptor binding domain thereof, wherein the chemokine includes but is not limited to CXC chemokines, CC chemokines, C chemokines and CX3C chemokines.

The term “chemokine activity” as used herein refers to the activities which chemokines possess or are able to exert in vivo, including but not limited to, chemotaxis of a variety of immune cells (including monocytes, macrophages, T cells, B cells, natural killer cells, dendritic cells and neutrophils, etc.).

The terms “protein”, “polypeptide” and “peptide” are used interchangeably herein when referring to a gene product comprising a polymer of amino acids.

The terms “subject,” “mammal,” and “patient” are used interchangeably. In some embodiments, the subject is a mammal. In some embodiments, the subject is a human. In some embodiments, the subject is a mouse, rat, rabbit, dog, donkey, or a laboratory test animal such as fruit fly, zebrafish, etc.

It is contemplated that the methods and compositions include exclusion of any of the embodiments described herein.

The terms “a” and “an” are defined as one or more unless this disclosure explicitly requires otherwise.

The term “substantially” is defined as being largely but not necessarily wholly what is specified (and include wholly what is specified) as understood by one of ordinary skill in the art. In any disclosed embodiment, the term “substantially” may be substituted with “within [a percentage] of” what is specified, where the percentage includes 0.1, 1, 5, and 10 percent.

The terms “comprise” (and any form of comprise, such as “comprises” and “comprising”), “have” (and any form of have, such as “has” and “having”), “include” (and any form of include, such as “includes” and “including”) and “contain” (and any form of contain, such as “contains” and “containing”) are open-ended linking verbs. As a result, the methods and systems of the present invention that “comprises,” “has,” “includes” or “contains” one or more elements possesses those one or more elements, but is not limited to possessing only those one or more elements. Likewise, an element of a method or system of the present invention that “comprises,” “has,” “includes” or “contains” one or more features possesses those one or more features, but is not limited to possessing only those one or more features.

The feature or features of one embodiment may be applied to other embodiments, even though not described or illustrated, unless expressly prohibited by this disclosure or the nature of the embodiments.

Any method or system of the present invention can consist of or consist essentially of—rather than comprise/include/contain/have—any of the described elements and/or features and/or steps. Thus, in any of the claims, the term “consisting of” or “consisting essentially of” can be substituted for any of the open-ended linking verbs recited above, in order to change the scope of a given claim from what it would otherwise be using the open-ended linking verb. A composition “consisting essentially of” the recited elements excludes any further active ingredients but does not exclude pharmaceutical excipients, buffers, structural components, etc.

### BRIEF DESCRIPTION OF THE DRAWINGS

The following drawings form part of the present specification and are included to further demonstrate certain aspects of the present invention. The invention may be better understood by reference to one or more of these drawings in combination with the detailed description of specific embodiments presented herein.

FIGS. 1A-E. Collagen binding domain (CBD) protein-fused IL-2 bind to collagen I and III with high affinity. (1A) Schematic of fusion of the vWF A3 recombinant protein to IL-2. (1B) IL-2 and CBD-IL-2 were analyzed by SDS-PAGE under reducing conditions with coomassie blue staining. (1C) Affinities (K_{D} values are shown) of IL-2 and CBD-IL-2 against collagen type I and collagen type III, recombinant mouse (rm) IL-2Rα were measured by ELISA. N.D.=not determined because of low signals. (1D) Graph with [concentrations] vs [signals] is shown (n=4). (1E) CTLL-2 cells are cultured in the presence of IL-2 or CBD-IL-2. After 48 hrs of culture, cell proliferation was analyzed. Two experimental repeats. Statistical analyses were done using ANOVA with Tukey's test. **p<0.01.

FIGS. 2A-B. CBD fusion reduces potential treatment-related toxicity of IL-2. (2A) 8×10⁵ MMTV-PyMT cells were inoculated on day 0. Fifty μg of DyLight 800-labeled CBD was injected i.v. when tumor volume reached 500 mm³. Fluorescence analysis of each organ revealed the bio-distribution of CBD protein after 48 hrs of injection. (2B) 5×10⁵ B16F10 cells were inoculated on day 0. CBD-IL-2 (12 μg) or IL-2 (6 μg) was injected i.v. on day 4. Blood serum was collected on day 5 and serum concentrations of IFNγ was measured (mean±SEM). Statistical analyses were done using ANOVA with Tukey's test. Two experimental repeats. *p<0.05; N.S.=not significant.

FIGS. 3A-B. CBD-IL-2 treatment reduces growth rate of B16F10 melanoma and CT26 colon carcinoma. (3A) 5×10⁵ B16F10 cells (3B) 5×10⁵ CT26 cells were inoculated on day 0. CBD-IL-2 (12 μg), IL-2 (6 μg) or PBS was administered i.v. on (3A) day 4 or (3B) day 5. Graphs depict tumor volume until the first mouse died. Tumor volumes are presented as mean±SEM. (3A) n=6 (3B) n=6. Two experimental repeats. Statistical analyses were done using ANOVA with Tukey's test for tumor size using the values of each day. *p<0.05; **p<0.01.

FIGS. 4A-4B. CBD fusion to IL-12 improves α-PD-L1 therapy. 5×10⁵ B16F10 cells were inoculated intradermally on day 0. 25 μg IL-12, 25 μg (IL-12 molar eq.) CBD-IL-12 or PBS was administered on days 7, 17 and 27 intravenously. 100 μg of α-PD-L1 or PBS was administered on days 8, 18 and 28 intraperitoneally. (A) Tumor growth rates and (B) survival rates are shown. PBS and α-PD-L1, n=7. α-PD-L1+IL-12 and α-PD-L1+CBD-IL-12, n=12. Tumor volumes are represented as mean±SEM. Statistical analysis for tumor growth curves was done using unpaired Mann-Whitney test (between α-PD-L1+IL-12 and α-PD-L1+CBD-IL-12). Statistical analysis for survival curve was done using Log-rank test. *p<0.05, **p<0.01.

FIGS. 5A-5B. CBD-IL-12 results in synergistic antitumor immunity with checkpoint inhibitors. 5×10⁵ B16F10 cells were inoculated intradermally on day 0. 25 μg (IL-12 molar eq.) CBD-IL-12 or PBS was administered on days 8, 13 and 18 intravenously. α-PD-L1 or α-PD-1+α-CTLA-4 (100 μg per antibody per dose) were administered on days 9, 14 and 19 intraperitoneally. (A) Tumor growth rates and (B) survival rates are shown. PBS, α-PD-L1, α-PD-1+α-CTLA-4, n=5. CBD-IL-12, α-PD-L1+CBD-IL-12, n=10. α-PD-1+α-CTLA-4+CBD-IL-12, n=11. Tumor volumes are represented as mean±SEM. Statistical analysis for survival curve was done using Log-rank test. *p<0.05, **p<0.01.

FIGS. 6A-6D. CBD-CCL4 fusion protein binds to collagen I and collagen III and maintains its activity. (A) WT CCL4 and CBD-CCL4 were analyzed by SDS-PAGE followed by Coomassie blue staining. (B,C) Affinity of CBD-CCL4 against (B) collagen I and (C) collagen III was measured by SPR. SPR chips were functionalized with collagen I (˜500 RU) and collagen III (˜700 RU), and the CBD-CCL4 was flowed over the chips at indicated concentrations. Curves represent the obtained specific responses (in resonance units (RU)) to CBD-CCL4. Experimental curves were fitted with 1:1 Langmuir fit model. Binding kinetics values [dissociation constants (K_{D}) and rate constants (kₒₙ and k_{off})] determined from the fitted curves are shown. (D) GPCR activation assay comparing signaling of WT CCL4 and CBD-CCL4 in ThP1 monocytes. EC₅₀ values were calculated using a non-linear dose-response curve fit model. Each point represents mean±SEM, n=3.

FIGS. 7A-7B. CBD-CCL4 fusion enhances blood plasma circulation time and improves tumor localization relative to WT CCL4. (A) Blood plasma pharmacokinetics was analyzed using DyLlight 800-labeled WT CCL4 or CBD-CCL4 in B16F10 melanoma. 4 d after intradermal inoculation with 5×10⁵ cells, mice were administered 25 μg WT CCL4 or molar equivalent CBD-CCL4 (25 μg CCL4 basis, or 93 μg CBD-CCL4) via i.v. injection. Blood was collected at the indicated time points, and plasma was separated and analyzed for CCL4concentration. Each point represents mean±SEM, n=4. (B) Biodistribution was analyzed using DyLlight 647-labeled WT CCL4 or CBD-CCL4 in EMT6 breast cancer. 5×10⁵ EMT6 cells were inoculated in the mammary fat pad. When the tumor volume reached 500 mm³, 25 μg WT CCL4 or molar equivalent CBD-CCL4 (25 μg CCL4 basis, or 93 μg CBD-CCL4) was given via i.v. injection. Florescent intensity in each tumor was measured using an IVIS imaging system, converted to percent injected dose using a known standard series, and normalized to the weight of the tumor. Each bar represents mean±SEM, n=3. **p<0.01.

FIGS. 8A-8H. CBD-CCL4 fusion recruits T cells, DCs, and improves efficacy of CPI therapy in B16F10 melanoma. Mice were intradermally injected with 5×10⁵ cells; 4 d later, mice were treated with WT CCL4 (25 μg given via i.v. injection) or molar equivalent CBD-CCL4 (25 μg CCL4 basis, or 93 μg CBD-CCL4, given via i.v. injection) in combination with CPI antibody therapy consisting of 100 μg each αPD-L1 and αCTLA4 given via i.p. injection. CPI therapy alone was administered as control. (A) Tumor growth was monitored over time until 10 d post tumor inoculation, at which point tumors were harvested and processed for flow cytometry analysis. (B-H) Immune cell composition was evaluated, where graphs depict the number of (B) CD45⁺ leukocytes, (C) CD103⁺ CD11c⁺ MHCII^{Hi} DCs, (D) CD8⁺ T cells, (E) NK1.1⁺ CD3⁻ NK cells, (F) total CD11c⁺ DCs, (G) CD4⁺ T cells, and (H) FoxP3⁺ CD25⁺ Tregs (of total CD4⁺ T cells). Bars represent mean±SEM, n=11-13. *p<0.05; **p<0.01; ***p<0.001; ****p<0.0001.

FIGS. 9A-9F. CBD-CCL4 combination therapy exhibits strong correlation between tumor growth and infiltration of CD103⁺ DCs and CD8⁺ T cells. (A-F) Regression analysis comparing the number of tumor-infiltrating cells with tumor volume was performed using the results obtained in FIG. 3. Correlations between (A) tumor volume and CD103⁺ CD11c⁺ MHCII^{Hi} DCs, (B) tumor volume and CD8⁺ T cells, (C) CD103⁺ CD11c⁺ MHCII^{Hi} DCs and CD8⁺ T cells, (D) tumor volume and NK1.1⁺ CD3⁻ NK cells (E) tumor volume and total CD11c⁺ DCs, and (F) tumor volume and total CD45⁺ leukocytes.

FIGS. 10A-10H. CBD-CCL4 combination treatment recruits T cells, DCs, and improves efficacy of CPI therapy in EMT6 breast cancer. Mice were subcutaneously injected with 5×10⁵ cells; 6 d and 9 d after inoculation, mice were treated with WT CCL4 (25 μg given via i.v. injection) or molar equivalent CBD-CCL4 (25 μg CCL4 basis, or 93 μg CBD-CCL4, given via i.v. injection) in combination with CPI antibody therapy consisting of 100 μg each αPD-L1 and αCTLA4 given via i.p. injection. CPI therapy alone was administered as control. (A) Tumor growth was monitored over time until 10 d post tumor inoculation, at which point tumors were harvested and processed for flow cytometry analysis. (B-H) Immune cell composition was evaluated, where graphs depict the number of (B) CD45⁺ leukocytes, (C) CD103⁺ CD11c⁺ MHCII^{Hi} DCs, (D) CD8α⁺ CD11c⁺ MHCII^{Hi} DCs (E) CD8⁺ T cells, (F) total CD11c⁺ DCs, (G) CD4⁺ T cells, and (H) FoxP3⁺ CD25⁺ Tregs (of total CD4⁺ T cells). Bars represent mean±SEM, n=7-9. *p<0.05; **p<0.01.

FIGS. 11A-11D. CBD-CCL4 combination treatment slows growth of established B16F10 melanoma and significantly prolongs survival. Mice were intradermally injected with 5×10⁵ cells; 7 d later, once tumor volume exceeded 50 mm³, mice were treated with CBD-CCL4 (25 μg CCL4 basis, or 93 μg CBD-CCL4, given via i.v. injection) in combination with CPI antibody therapy consisting of 100 μg each αPD-L1 and αCTLA4 given via i.p. injection. CPI therapy alone was administered as comparison. (A) Tumor growth curves until the first mouse died, (B) survival curves, and individual growth curves for (C) CPI therapy alone or (D) CBD-CCL4 combination therapy are shown. Graphs depict mean±SEM, n=5. **p<0.01; ***p<0.001; ****p<0.0001.

FIGS. 12A-12B. CBD-CCL4 therapy synergizes with αPD-1 immunotherapy to slow growth of CT26 and MC38 colon carcinoma. Mice were intradermally injected with 5×10⁵ CT26 or MC38 cells; 5 d after inoculation, mice were treated with WT CCL4 (25 μg given via i.v. injection) or molar equivalent CBD-CCL4 (25 μg CCL4 basis, or 93 μg CBD-CCL4 given via i.v. injection) in combination with 100 μg αPD-1 given via i.p. injection. 100 μg αPD-1 alone was administered as control. Graphs depict growth curves of (A) CT26 and (B) MC38 tumor models until the first mouse died. Bars represent mean±SEM, n=5. *p<0.05; **p<0.01; ****p<0.0001.

FIG. 13A-13D. CBD-CCL4 in combination with CPI therapy slows growth of spontaneous MMTV-PyMT breast cancer. MMTV-PyMT mice were monitored until total tumor burden reached 100 mm³. At this point, mice were treated with CBD-CCL4 (25 μg CCL4 basis, or 93 μg CBD-CCL4, given via i.v. injection) in combination with CPI antibody therapy consisting of 100 μg each αPD-L1 and αCTLA4 given via i.p. injection. CPI therapy alone was administered as comparison. Identical dosing was given 7 d and 14 d after the initial treatment. (A) Tumor growth curves until first two mice died, (B) survival curves, and individual growth curves for (C) CPI therapy alone or (D) CBD-CCL4 combination therapy are shown. Graphs depict mean±SEM, n=6. *p<0.05; **p<0.01.

### DETAILED DESCRIPTION

Cytokines and chemokines, referred to herein generally as cytokines, have been shown to exhibit considerable anti-tumor activity, but previous studies have reported instances of narrow therapeutic windows and/or treatment-related adverse events. The methods and compositions described herein provide for localized therapy with a cytokine that is retained intra- or peri-tumorally, limiting systemic exposure and reducing side-effects that, in some cases, can be so severe that either the therapy has to be discontinued or an effective dose is not achievable. The examples provided herein demonstrate enhanced tumor tissue retention and lower concentrations in blood plasma following ECM-affinity peptide conjugation, reducing systemic side effects. Intravenous (iv) injections of the compositions described herein significantly delayed tumor growth, prolonging survival compared to controls in mouse models for melanoma and colon cancer. This simple and translatable approach of engineered ECM-binding cytokines represent a novel approach in cancer therapy.

IL-2 was used as a representative cytokine to demonstrate the concept of localizing cytokines to the tumor vasculature via an ECM affinity peptides, e.g., collagen binding domains (CBDs). Cytokine immunotherapy with interleukin-2 (IL-2) exhibits considerable antitumor activity in animal models and the clinic. The inventors have tested the use of CBDs to target leaky vasculature by engineered a fusion protein of IL-2 to a CBD from the von Willebrand Factor (vWF) A3 domain; the fusion protein can be administered intravenously and target the tumor microenvironment via its leaky vasculature. This demonstrates the use of such fusions/conjugates to access the tumor stroma. Below in the examples it is shown that CBD protein localizes to tumor tissue; CBD-IL-2 showed a lower serum concentrations after injection; and CBD-IL-2 significantly delayed tumor growth compared to wild-type IL-2 in murine melanoma and colon cancer models. This simple and translatable approach of an engineered collagen-binding cytokine presents a novel approach to cancer immunotherapeutics.

Blood vessels are normally lined with tightly linked cells, called endothelial cells, that form an impermeable barrier. Vascular leak occurs when small blood vessels, generally a capillary or venule, become leaky and release fluid. Vascular leak can occur under a variety of conditions, including tumor microenvironment or chronic inflammation, and can affect almost all the organ beds. Methods and compositions for treatment of or targeting of araes of vacular permeability in chronic inflammation associated disorders such as insulin resistance, diabetes, cardiovascular disease, metabolic disorders, and cancer are contemplated

In the treatment of a disease associated with an increase of vascular permeability a collagen binding domain described herein may be effectively used as a pharmaceutical composition for preventing and/or treating such a disease. In this aspect, the pharmaceutical composition for preventing and/or treating a disease associated with vascular permeability can include the localization of a therapeutic to an area of vascular leakage or permeability. The therapeutic can be a peptide, polypeptide, compound, antibody, genes (for example, antisense oligonucleotide, siRNA, shRNA, microRNA, and the like), aptamers, therapeutic cells, radiopharmaceutical drugs.

### I. CYTOKINES AND CHEMOKINES

Cytokines are a group of proteins that cells release upon excitation (only very few cytokines are expressed on cell membranes). Cytokines produced by cells can affect target cells nearby or through blood circulation at very low concentration. They have broad functions on promoting growth, differentiation, and activation of target cells. Many cytokines can target immune cells and play a role in immune response. Based on structural and functional differences, cytokines may be broadly divided into chemokines, interleukins, growth factors, transforming growth factors, colony stimulating factors, tumor necrosis factors, and interferons.

Interleukins are a group of cytokines first identified to be expressed by white blood cells (leukocytes). The function of the immune system depends in a large part on interleukins. The majority of interleukins are synthesized by helper CD4 T lymphocytes, as well as monocytes, macrophages, and endothelial cells. Generally interleukins promote the development and differentiation of T and B lymphocytes, and hematopoietic cells. Interleukin 2 (IL-2) is classified into the hematopoietin family, the family including a number of cell growth-related hormones or other cytokines. Functions of IL-2 include: regulating the maturation and differentiation of T cells, stimulating proliferation and antibody secretion of B cells, promoting cytotoxicity of natural killer cells, and activating monocytes and macrophages. IL-2 can also stimulate T cells and B cells to continue expressing MHC, and also stimulate natural killer cells to produce several different cytokines, including TNF-α, IFN-γ and granulocyte/macrophage colony stimulating factor (GM-CSF). Studies have shown that IL-2 has anti-tumor and vaccine-enhancing effects.

Chemokines are a group of cytokines able to attract leukocytes. Chemokines are typically positively charged, secreted proteins having small molecular weights. Their main function is to attract immune cells to a region having tissue injuries or pathogen infection, allowing leukocytes to subsequently perform phagocytosis or elicit inflammation against pathogens at this specific site. Leukocytes attracted by chemokines may include neutrophils, monocytes/macrophages, natural killer cells, dendritic cells and other leukocytes, which are of innate immunity; and T lymphocytes (T cells) or B lymphocytes (B cells) of adaptive immunity. Typically chemokines have four highly conserved cysteine (C) forming disulfide bonds to stabilize their structure. Based on different numbers of amino acids between the first two cysteines and the procession of the first cysteine or not, they may be classified into four subfamilies of CXC (or α), CC (or β), C (or γ) and CX3C. Stromal cell-derived factor-1 (SDF-1) is classified into the CXC subfamily of chemokines, and is also known as CXC ligand 12 (CXCL12).

In certain embodiments the cytokine is IL-2. Cancer immunotherapy has shown considerable therapeutic effects in the clinic. As a cytokine drug, interleukin-2 (IL-2: aldesleukin) has been approved by the US Food and Drug Administration (FDA) (Jiang et al., Oncoimmunology 5:e1163462, 2016) for treatment of advanced melanoma and renal cell cancer. Aldesleukin shows high response rates, prolonging survival, whereas it has a narrow therapeutic window due to induction of severe adverse events (Rosenberg et al., N Engl J Med 313:1485-1492, 1985). IL-2 is mainly secreted from CD4⁺ T cells and, to lesser extent, CD8⁺ T cells and natural killer (NK) cells (Boyman and Sprent, Nature reviews. Immunology 12:180-190, 2012). IL-2 induces proliferation and activation of T cells, B cells, and NK cells (Boyman and Sprent, Nature reviews. Immunology 12:180-190). Although IL-2 activates the immune system at high concentrations, IL-2 reverses its effect at low concentration to induce and maintain regulatory T cells (Tregs), which are immunosuppressive cells (Jiang et al., Oncoimmunology 5:e1163462, 2016). Because of its fast half-life in the blood, engineering of IL-2 fusion protein to increase its half-life has been proposed and shows higher efficacy compared to its wild-type form (Zhu et al., Cancer Cell 27:489-501, 2015).

In another embodiment the cytokine can be selected from IL-12, IL-15, IL-21, CC14, CCL21, CXCL9, CXCL10, VEGF-C, or other cytokine having anti-tumor properties.

Human IL-15 has an amino acid sequence of-
(SEQ ID NO: 16)
MNWVNVISDLKKIEDLIQSMHIDATLYTESDVHPSCKVTAMKCFLLELQV
ISLESGDASIHDTVENLIILANNSLSSNGNVTESGCKECEELEEKNIKEF
LQSFVHIVQMFINTS.
Mouse IL-15 has an amino acid sequence of-
(SEQ ID NO: 17)
MNWIDVRYDLEKIESLIQSIHIDTTLYTDSDFHPSCKVTAMNCFLLELQV
ILHEYSNMTLNETVRNVLYLANSTLSSNKNVAESGCKECEELEEKTFTEF
LQSFIRIVQMFINTS.
Human IL-21 has an amino acid sequence of
(SEQ ID NO: 18)
MQDRHMIRMRQLIDIVDQLKNYVNDLVPEFLPAPEDVETNCEWSAFSCFQ
KAQLKSANTGNNERIINVSIKKLKRKPPSTNAGRRQKHRLTCPSCDSYEK
KPPKEFLERFKSLLQKMIHQHLSSRTHGSEDS.
Mouse IL-21 has an amino acid sequence of-
(SEQ ID NO: 19)
MHKSSPQGPDRLLIRLRHLIDIVEQLKIYENDLDPELLSAPQDVKGHCEH
AAFACFQKAKLKPSNPGNNKTFIIDLVAQLRRRLPARRGGKKQKHIAKCP
SCDSYEKRTPKEFLERLKWLLQKMIHQHLS.
Human IL-12 p35 Subunit has an amino acid
sequence of-
(SEQ ID NO: 20)
RNLPVATPDPGMFPCLHHSQNLLRAVSNMLQKARQTLEFYPCTSEEIDHE
DITKDKTSTVEACLPLELTKNESCLNSRETSFITNGSCLASRKTSFMMAL
CLSSIYEDLKMYQVEFKTMNAKLLMDPKRQIFLDQNMLAVIDELMQALNF
NSETVPQKSSLEEPDFYKTKIKLCILLHAFRIRAVTIDRVMSYLNAS.
Human IL-12 p40 Subunit has an amino acid
sequence of-
(SEQ ID NO: 21)
IWELKKDVYVVELDWYPDAPGEMVVLTCDTPEEDGITWTLDQSSEVLGSG
KTLTIQVKEFGDAGQYTCHKGGEVLSHSLLLLHKKEDGIWSTDILKDQKE
PKNKTFLRCEAKNYSGRFTCWWLTTISTDLTESVKSSRGSSDPQGVTCGA
ATLSAERVRGDNKEYEYSVECQEDSACPAAEESLPIEVMVDAVHKLKYEN
YTSSFFIRDIIKPDPPKNLQLKPLKNSRQVEVSWEYPDTWSTPHSYFSLT
FCVQVQGKSKREKKDRVFTDKTSATVICRKNASISVRAQDRYYSSSWSEW
ASVPCS.
Mouse IL-12 p35 Subunit has an amino acid
sequence of-
(SEQ ID NO: 22)
RVIPVSGPARCLSQSRNLLKTTDDMVKTAREKLKHYSCTAEDIDHEDITR
DQTSTLKTCLPLELHKNESCLATRETSSTTRGSCLPPQKTSLMMTLCLGS
IYEDLKMYQTEFQAINAALQNHNHQQIILDKGMLVAIDELMQSLNHNGET
LRQKPPVGEADPYRVKMKLCILLHAFSTRVVTINRVMGYLSSA.
Mouse IL-12 p40 Subunit has an amino acid
sequence of-
(SEQ ID NO: 23)
MWELEKDVYVVEVDWTPDAPGETVNLTCDTPEEDDITWTSDQRHGVIGSG
KTLTITVKEFLDAGQYTCHKGGETLSHSHLLLHKKENGIWSTEILKNEKN
KTFLKCEAPNYSGRFTCSWLVQRNMDLKFNIKSSSSSPDSRAVTCGMASL
SAEKVTLDQRDYEKYSVSCQEDVTCPTAEETLPIELALEARQQNKYENYS
TSFFIRDIIKPDPPKNLQMKPLKNSQVEVSWEYPDSWSTPHSYFSLKFFV
RIQRKKEKMKETEEGCNQKGAFLVEKTSTEVQCKGGNVCVQAQDRYYNSS
CSKWACVPCRVRS.
Human CCL4 has an amino acid sequence of-
(SEQ ID NO: 24)
APMGSDPPTACCFSYTARKLPHNFVVDYYETSSLCSQPAVVFQTKRGKQV
CADPSESWVQ EYVYDLELN.
Mouse CCL4 has an amino acid sequence of-
(SEQ ID NO: 25)
APMGSDPPTSCCFSYTSRQLHRSFVMDYYETSSLCSKPAVVFLTKRGRQI
CANPSEPWVTEYMSDLELN.
Human CCL21 has an amino acid sequence of-
(SEQ ID NO: 26)
SDGGAQDCCLKYSQRKIPAKVVRSYRKQEPSLGCSIPAILFLPRKRSQAE
LCADPKELWVQQLMQHLDKTPSPQKPAQGCRKDRGASKTGKKGKGSKGCR
KTERSQTPKGP.
Mouse CCL21 has an amino acid sequence of-
(SEQ ID NO: 27)
SDGGGQDCCLKYSQKKIPYSIVRGYRKQEPSLGCPIPAILFLPRKHSKPE
LCANPEEGWVQNLMRRLDQPPAPGKQSPGCRKNRGTSKSGKKGKGSKGCK
RTEQTQPSRG.
Human CXCL9 has an amino acid sequence of-
(SEQ ID NO: 28)
TPVVRKGRCSCISTNQGTIHLQSLKDLKQFAPSPSCEKIEIIATLKNGVQ
TCLNPDSADVKELIKKWEKQVSQKKKQKNGKKHQKKKVLKVRKSQRSRQK
KTT.
Mouse CXCL9 has an amino acid sequence of-
(SEQ ID NO: 29)
TLVIRNARCSCISTSRGTIHYKSLKDLKQFAPSPNCNKTEIIATLKNGDQ
TCLDPDSANVKKLMKEWEKKINQKKKQKRGKKHQKNMKNRKPKTPQSRRR
SRKTT.
Human CXCL10 has an amino acid sequence of-
(SEQ ID NO: 30)
VPLSRTVRCTCISISNQPVNPRSLEKLEIIPASQFCPRVEIIATMKKKGE
KRCLNPESKAIKNLLKAVSKERSKRSP.
Mouse CXCL10 has an amino acid sequence of-
(SEQ ID NO: 31)
IPLARTVRCNCIHIDDGPVRMRAIGKLEIIPASLSCPRVEIIATMKKNDE
QRCLNPESKTIKNLMKAFSQKRSKRAP.
Human VEGF-C has an amino acid sequence of-
(SEQ ID NO: 32)
AHYNTEILKSIDNEWRKTQCMPREVCIDVGKEFGVATNTFFKPPCVSVYR
CGGCCNSEGLQCMNTSTSYLSKTLFEITVPLSQGPKPVTISFANHTSCRC
MSKLDVYRQVHSIIRR.
Mouse VEGF-C has an amino acid sequence of-
(SEQ ID NO: 33)
TEETIKFAAAHYNTEILKSIDNEWRKTQCMPREVCIDVGKEFGVATNTFF
KPPCVSVYRCGGCCNSEGLQCMNTSTSYLSKTLFEITVPLSQGPKPVTIS
FANHTSCRCMSKLDVYRQVHSIIRR.
Mouse IFNβ has an amino acid sequence of-
(SEQ ID NO: 39)
MNNRWILHAAFLLCFSTTALSINYKQLQLQERTNIRKCQELLEQLNGKIN
LTYRADFKIPMEMTEKMQKSYTAFAIQEMLQNVFLVFRNNFSSTGWNETI
VVRLLDELHQQTVFLKTVLEEKQEERLTWEMSSTALHLKSYYWRVQRYLK
LMKYNSYAWMVVRAEIFRNFLIIRRLTRNFQN.
Human IFNβ has an amino acid sequence of-
(SEQ ID NO: 40)
MSYNLLGFLQRSSNFQCQKLLWQLNGRLEYCLKDRMNFDIPEEIKQLQQF
QKEDAALTIYEMLQNIFAIFRQDSSSTGWNETIVENLLANVYHQINHLKT
VLEEKLEKEDFTRGKLMSSLHLKRYYGRILHYLKAKEYSHCAWTIVRVEI
LRNFYFINRLTGYLRN.
Mouse IFNα2 has an amino acid sequence of-
(SEQ ID NO: 41)
CDLPHTYNLRNKRALKVLAQMRRLPFLSCLKDRQDFGFPLEKVDNQQIQK
AQAIPVLRDLTQQTLNLFTSKASSAAWNTTLLDSFCNDLHQQLNDLQTCL
MQQVGVQEPPLTQEDALLAVRKYFHRITVYLREKKHSPCAWEVVRAEVWR
ALSSSVNLLPRLSEEKE.
Human IFNα2 has an amino acid sequence of-
(SEQ ID NO: 42)
MALTFALLVALLVLSCKSSCSVGCDLPQTHSLGSRRTLMLLAQMRKISLF
SCLKDRHDFGFPQEEFGNQFQK.
Mouse XCL1 has an amino acid sequence of-
(SEQ ID NO: 43)
VGTEVLEESSCVNLQTQRLPVQKIKTYIIWEGAMRAVIFVTKRGLKICAD
PEAKWVKAAIKTVDGRASTRKNMAETVPTGAQRSTSTAITLTG.
Human XCL1 has an amino acid sequence of-
(SEQ ID NO: 44)
GSEVSDKRTCVSLTTQRLPVSRIKTYTITEGSLRAVIFITKRGLKVCADP
QATWVRDVVRSMDRKSNTRN NMIQTKPTGTQQSTNTAVTLTG.
Mouse IL-15 super agonist has an amino acid
sequence of-
(SEQ ID NO: 45)
GTTCPPPVSIEHADIRVKNYSVNSRERYVCNSGFKRKAGTSTLIECVINK
NTNVAHWTTPSLKCIRDPSLAHYSPVPTVVTPKVTSQPESPSPSAKEPEA
SGGSGGGGSGGGSGGGGSLQNWIDVRYDLEKIESLIQSIHIDTTLYTDSD
FHPSCKVTAMNCFLLELQVILHEYSNMTLNETVRNVLYLANSTLSSNKNV
AESGCKECEELEEKTFTEFLQSFIRIVQMFINTS.
Human IL-15 super agonist has an amino acid
sequence of-
(SEQ ID NO: 46)
ITCPPPMSVEHADIWVKSYSLYSRERYICNSGFKRKAGTSSLTECVLNKA
TNVAHWTTPSLKCIRDVDDKTHTCPPCPAPELLGGPSVFLFPPKPKDTLM
ISRTPEVTCVVVDVSHEDPEVKFNWYVDGVEVHNAKTKPREEQYNSTYRV
VSVLTVLHQDWLNGKEYKCKVSNKALPAPIEKTISKAKGQPREPQVYTLP
PSREEMTKNQVSLTCLVKGFYPSDIAVEWESNGQPENNYKTTPPVLDSDG
SFFLYSKLTVDKSRWQQGNVFSCSVMHEALHNHYTQKSLSLSPGKGGGGS
GGGGSGGGGSNWVNVISDLKKIEDLIQSMHIDATLYTESDVHPSCKVTAM
KCFLLELQVISLESGDASIHDTVENLIILANDSLSSNGNVTESGCKECEE
LEEKNIKEFLQSFVHIVQMFINTS.

### II. ECM-AFFINITY PEPTIDES

Collagen is an extracellular matrix (ECM)-protein that regulates a variety of cellular biological functions, such as proliferation, differentiation, and adhesion in both normal and tumor tissue (Ricard-Blum, Cold Spring Harb Perspect Biol 3:a004978, 2011). Collagen is the most abundant protein in the mammalian body and exists in almost all tissues in one or more of 28 isoforms (Ricard-Blum, Cold Spring Harb Perspect Biol 3:a004978, 2011). The blood vessel sub-endothelial space is rich in collagen. Because of its insolubility under physiological conditions, collagen barely exists within the blood (Dubois et al., Blood 107:3902-06, 2006; Bergmeier and Hynes, Cold Spring Harb Perspect Biol 4:a005132, 2012). Tumor vasculature is reported to be permeable due to an abnormal structure (Nagy et al., British journal of cancer 100:865, 2009). Thus, with its leaky vasculature, collagen is exposed in the tumor (Liang et al., Journal of controlled release 209:101-109, 2015; Liang et al., Sci Rep 6:18205, 2016; Yasunaga et al., Bioconjugate chemistry 22:1776-83, 2011; Xu et al. The Journal of cell biology 154:1069-80, 2001; Swartz and Lund, Nat Rev Cancer 12:210-19). Also, tumor tissue contains increased amounts of collagen compared to normal tissues (Zhou et al. J Cancer 8:1466-76, 2017; Provenzano et al. BMC Med 6:11, 2008).

von Willebrand factor (vWF) is a blood coagulation factor and binds to both type I and type III collagen, and the adhesion receptor GPIbα on blood platelets (Lenting et al., Journal of thrombosis and haemostasis:JTH 10:2428-37, 2012; Shahidi Advances in experimental medicine and biology 906:285-306, 2017). When injured, collagen beneath endothelial cells is exposed to blood plasma, and vWF-collagen binding initiates the thrombosis cascade (Shahidi Advances in experimental medicine and biology 906:285-306, 2017; Wu et al. Blood 99:3623-28, 2002). The vWF A domain has the highest affinity against collagen among reported non-bacterial origin proteins/peptides (Addi et al., Tissue Engineering Part B: Reviews, 2016). Particularly within the A domain, the A3 domain of vWF has been reported as a collagen binding domain (CBD) (Ribba et al. Thrombosis and haemostasis 86:848-54, 2001). As described above, the inventors contemplated that a fusion protein with the vWF A3 CBD may achieve targeted cytokine immunotherapy even when injected systemically due to exposure of collagen via the leaky tumor vasculature.

Embodiments of the disclosure relate to ECM-affinity peptides. In some embodiments, the ECM-affinity peptide is a peptide from von Willebrand factor (vWF). The sequence of human vWF comprises the following:

(SEQ ID NO: 13)
MIPARFAGVLLALALILPGTLCAEGTRGRSSTARCSLFGSDFVNTFDGSM
YSFAGYCSYLLAGGCQKRSFSIIGDFQNGKRVSLSVYLGEFFDIHLFVNG
TVTQGDQRVSMPYASKGLYLETEAGYYKLSGEAYGFVARIDGSGNFQVLL
SDRYFNKTCGLCGNFNIFAEDDFMTQEGTLTSDPYDFANSWALSSGEQWC
ERASPPSSSCNISSGEMQKGLWEQCQLLKSTSVFARCHPLVDPEPFVALC
EKTLCECAGGLECACPALLEYARTCAQEGMVLYGWTDHSACSPVCPAGME
YRQCVSPCARTCQSLHINEMCQERCVDGCSCPEGQLLDEGLCVESTECPC
VHSGKRYPPGTSLSRDCNTCICRNSQWICSNEECPGECLVTGQSHFKSFD
NRYFTFSGICQYLLARDCQDHSFSIVIETVQCADDRDAVCTRSVTVRLPG
LHNSLVKLKHGAGVAMDGQDVQLPLLKGDLRIQHTVTASVRLSYGEDLQM
DWDGRGRLLVKLSPVYAGKTCGLCGNYNGNQGDDFLTPSGLAEPRVEDFG
NAWKLHGDCQDLQKQHSDPCALNPRMTRFSEEACAVLTSPTFEACHRAVS
PLPYLRNCRYDVCSCSDGRECLCGALASYAAACAGRGVRVAWREPGRCEL
NCPKGQVYLQCGTPCNLTCRSLSYPDEECNEACLEGCFCPPGLYMDERGD
CVPKAQCPCYYDGEIFQPEDIFSDHHTMCYCEDGFMHCTMSGVPGSLLPD
AVLSSPLSHRSKRSLSCRPPMVKLVCPADNLRAEGLECTKTCQNYDLECM
SMGCVSGCLCPPGMVRHENRCVALERCPCFHQGKEYAPGETVKIGCNTCV
CRDRKWNCTDHVCDATCSTIGMAHYLTFDGLKYLFPGECQYVLVQDYCGS
NPGTFRILVGNKGCSHPSVKCKKRVTILVEGGEIELFDGEVNVKRPMKDE
THFEVVESGRYIILLLGKALSVVWDRHLSISVVLKQTYQEKVCGLCGNFD
GIQNNDLTSSNLQVEEDPVDFGNSWKVSSQCADTRKVPLDSSPATCHNNI
MKQTMVDSSCRILTSDVFQDCNKLVDPEPYLDVCIYDTCSCESIGDCACF
CDTIAAYAHVCAQHGKVVTWRTATLCPQSCEERNLRENGYECEWRYNSCA
PACQVTCQHPEPLACPVQCVEGCHAHCPPGKILDELLQTCVDPEDCPVCE
VAGRRFASGKKVTLNPSDPEHCQICHCDVVNLTCEACQEPGGLVVPPTDA
PVSPTTLYVEDISEPPLHDFYCSRLLDLVFLLDGSSRLSEAEFEVLKAFV
VDMMERLRISQKWVRVAVVEYHDGSHAYIGLKDRKRPSELRRIASQVKYA
GSQVASTSEVLKYTLFQIFSKIDRPEASRITLLLMASQEPQRMSRNFVRY
VQGLKKKKVIVIPVGIGPHANLKQIRLIEKQAPENKAFVLSSVDELEQQR
DEIVSYLCDLAPEAPPPTLPPDMAQVTVGPGLLGVSTLGPKRNSMVLDVA
FVLEGSDKIGEADFNRSKEFMEEVIQRMDVGQDSIHVTVLQYSYMVTVEY
PFSEAQSKGDILQRVREIRYQGGNRTNTGLALRYLSDHSFLVSQGDREQA
PNLVYMVTGNPASDEIKRLPGDIQVVPIGVGPNANVQELERIGWPNAPIL
IQDFETLPREAPDLVLQRCCSGEGLQIPTLSPAPDCSQPLDVILLLDGSS
SFPASYFDEMKSFAKAFISKANIGPRLTQVSVLQYGSITTIDVPWNVVPE
KAHLLSLVDVMQREGGPSQIGDALGFAVRYLTSEMHGARPGASKAVVILV
TDVSVDSVDAAADAARSNRVTVFPIGIGDRYDAAQLRILAGPAGDSNVVK
LQRIEDLPTMVTLGNSFLHKLCSGFVRICMDEDGNEKRPGDVWTLPDQCH
TVTCQPDGQTLLKSHRVNCDRGLRPSCPNSQSPVKVEETCGCRWTCPCVC
TGSSTRHIVTFDGQNFKLTGSCSYVLFQNKEQDLEVILHNGACSPGARQG
CMKSIEVKHSALSVELHSDMEVTVNGRLVSVPYVGGNMEVNVYGAIMHEV
RFNHLGHIFTFTPQNNEFQLQLSPKTFASKTYGLCGICDENGANDFMLRD
GTVTTDWKTLVQEWTVQRPGQTCQPILEEQCLVPDSSHCQVLLLPLFAEC
HKVLAPATFYAICQQDSCHQEQVCEVIASYAHLCRTNGVCVDWRTPDFCA
MSCPPSLVYNHCEHGCPRHCDGNVSSCGDHPSEGCFCPPDKVMLEGSCVP
EEACTQCIGEDGVQHQFLEAWVPDHQPCQICTCLSGRKVNCTTQPCPTAK
APTCGLCEVARLRQNADQCCPEYECVCDPVSCDLPPVPHCERGLQPTLTN
PGECRPNFTCACRKEECKRVSPPSCPPHRLPTLRKTQCCDEYECACNCVN
STVSCPLGYLASTATNDCGCTTTTCLPDKVCVHRSTIYPVGQFWEEGCDV
CTCTDMEDAVMGLRVAQCSQKPCEDSCRSGFTYVLHEGECCGRCLPSACE
VVTGSPRGDSQSSWKSVGSQWASPENPCLINECVRVKEEVFIQQRNVSCP
QLEVPVCPSGFQLSCKTSACCPSCRCERMEACMLNGTVIGPGKTVMIDVC
TTCRCMVQVGVISGFKLECRKTTCNPCPLGYKEENNTGECCGRCLPTACT
IQLRGGQIMTLKRDETLQDGCDTHFCKVNERGEYFWEKRVTGCPPFDEHK
CLAEGGKIMKIPGTCCDTCEEPECNDITARLQYVKVGSCKSEVEVDIHYC
QGKCASKAMYSIDINDVQDQCSCCSPTRTEPMQVALHCTNGSVVYHEVLN
AMECKCSPRKCSK.

In some embodiments, the peptide is from the vWF A3 domain. The vWF A3 domain is derived from the human sequence, residues 1670-1874 (907-1111 of mature vWF) and has the following sequence: CSGEGLQIPTLSPAPDCSQPLDVILLLDGSSSFPASYFDEMKSFAKAFISKANIGPRLTQVS VLQYGSITTIDVPWNVVPEKAHLLSLVDVMQREGGPSQIGDALGFAVRYLTSEMHGAR PGASKAVVILVTDVSVDSVDAAADAARSNRVTVFPIGIGDRYDAAQLRILAGPAGDSNV VKLQRIEDLPTMVTLGNSFLHKLCSG (SEQ ID NO:3). Examples of peptides include all or part (i.e., a segment) of SEQ ID NO:13 or all or part of SEQ ID NO:3. In some embodiments, the peptide is from the vWF A1 domain. The vWF A1 domain is derived from the human sequence, residues 1237-1458 (474-695 of mature vWF) and has the following sequence:

(SEQ ID NO: 11)
CQEPGGLVVPPTDAPVSPTTLYVEDISEPPLHDFYCSRLLDLVFLLDGSS
RLSEAEFEVLKAFVVDMMERLRISQKWVRVAVVEYHDGSHAYIGLKDRKR
PSELRRIASQVKYAGSQVASTSEVLKYTLFQIFSKIDRPEASRITLLLMA
SQEPQRMSRNFVRYVQGLKKKKVIVIPVGIGPHANLKQIRLIEKQAPENK
AFVLSSVDELEQQRDEIVSYLC.

In certain embodiments the ECM-affinity peptide comprises all or part of decorin amino acid sequence. Decorin collagen binding domain has the following amino acid sequence:

Human decorin
(SEQ ID NO: 15)
GPFQQRGLFDFMLEDEASGIGPEVPDDRDFEPSLGPVCPFRCQCHLRVVQ
CSDLGLDKVPKDLPPDTTLLDLQNNKITEIKDGDFKNLKNLHALILVNNK
ISKVSPGAFTPLVKLERLYLSKNQLKELPEKMPKTLQELRAHENEITKVR
KVTFNGLNQMIVIELGTNPLKSSGIENGAFQGMKKLSYIRIADTNITSIP
QGLPPSLTELHLDGNKISRVDAASLKGLNNLAKLGLSFNSISAVDNGSLA
NTPHLRELHLDNNKLTRVPGGLAEHKYIQVVYLHNNNISVVGSSDFCPPG
HNTKKASYSGVSLFSNPVQYWEIQPSTFRCVYVRSAIQLGNYK

In some embodiments, the ECM-affinity peptide comprises a peptide from PlGF-2. PlGF-2 has the following sequence:

(SEQ ID NO: 4)
MPVMRLFPCFLQLLAGLALPAVPPQQWALSAGNGSSEVEVVPFQEVWGRS
YCRALERLVDVVSEYPSEVEHMFSPSCVSLLRCTGCCGDENLHCVPVETA
NVTMQLLKIRSGDRPSYVELTFSQHVRCECRPLREKMKPERRRPKGRGKR
RREKQRPTDCHLCGDAVPRR.

Exemplary PlGF-2 ECM affinity peptides include:

(SEQ ID NO: 5)
RRRPKGRGKRRREKQRPTDCHLCGDAVPRR;
(SEQ ID NO: 1)
RRRPKGRGKRRREKQRPTDCHL;
(SEQ ID NO: 6)
RRPKGRGKRRREKQRPTD;
(SEQ ID NO: 7)
RRRPKGRGKRRREKQ;
(SEQ ID NO: 8)
GKRRREKQ;
(SEQ ID NO: 9)
RRRPKGRG;
and
(SEQ ID NO: 10)
RRKTKGKRKRSRNSQTEEPHP.

In some embodiments, the ECM-affinity peptide is a peptide from CXCL-12γ. The sequence of CXCL-12γ is the following: CXCL-12γ: KPVSLSYRCPCRFFESHVARANVKHLKILNTPNCALQIVARLKNNNRQVCIDPKLKWIQ EYLEKALNKGRREEKVGKKEKIGKKKRQKKRKAAQKRKN (SEQ ID NO:12). An exemplary peptide includes all or part of SEQ ID NO:12 and the following peptide: GRREEKVGKKEKIGKKKRQKKRKAAQKRKN (SEQ ID NO:2).

The ECM-affinity peptide may be a peptide with 75, 76, 77, 78, 79, 80, 81, 82, 83, 84,85, 86, 87, 88, 89, 90, 91, 92, 93, 94, 95, 96, 97, 98, 99, or 100% identity (or any derivable range therein) to an ECM or CBD peptide or fragment of the peptides described above. The peptide or polypeptide may have one or more conservative or non-conservative substitutions. Substitutional variants typically contain the exchange of one amino acid for another at one or more sites within the protein, and may be designed to modulate one or more properties of the polypeptide, with or without the loss of other functions or properties. Substitutions may be conservative, that is, one amino acid is replaced with one of similar shape and charge. Conservative substitutions are well known in the art and include, for example, the changes of: alanine to serine; arginine to lysine; asparagine to glutamine or histidine; aspartate to glutamate; cysteine to serine; glutamine to asparagine; glutamate to aspartate; glycine to proline; histidine to asparagine or glutamine; isoleucine to leucine or valine; leucine to valine or isoleucine; lysine to arginine; methionine to leucine or isoleucine; phenylalanine to tyrosine, leucine or methionine; serine to threonine; threonine to serine; tryptophan to tyrosine; tyrosine to tryptophan or phenylalanine; and valine to isoleucine or leucine. Alternatively, substitutions may be non-conservative such that a function or activity of the polypeptide is affected. Non-conservative changes typically involve substituting a residue with one that is chemically dissimilar, such as a polar or charged amino acid for a nonpolar or uncharged amino acid, and vice versa.

The polypeptides described herein may include 1, 2, 3, 4, 5, 6, 7, 8, 9, 10, 11, 12, 13, 14, 15, or more (or any derivable range therein) variant amino acids within at least, or at most 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 201, 202, 203, 204, 205, 206, 207, 208, 209, 210, 211, 212, 213, 214, 215, 216, 217, 218, 219, 220, 221, 222, 223, 224, 225, 226, 227, 228, 229, 230, 231, 232, 233, 234, 235, 236, 237, 238, 239, 240, 241, 242, 243, 244, 245, 246, 247, 248, 249, 250, 300, 400, 500, 550, 1000 or more contiguous amino acids, or any range derivable therein, of a peptide or polypeptide of the disclosure.

A polypeptide segment or fragment as described herein may include 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 201, 202, 203, 204, 205, 206, 207, 208, 209, 210, 211, 212, 213, 214, 215, 216, 217, 218, 219, 220, 221, 222, 223, 224, 225, 226, 227, 228, 229, 230, 231, 232, 233, 234, 235, 236, 237, 238, 239, 240, 241, 242, 243, 244, 245, 246, 247, 248, 249, 250, 300, 400, 500, 550, 1000 or more contiguous amino acids, or any range derivable therein of a peptide or polypeptide of the disclosure.

The polypeptides described herein may be of a fixed length of at least, at most, or exactly 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 201, 202, 203, 204, 205, 206, 207, 208, 209, 210, 211, 212, 213, 214, 215, 216, 217, 218, 219, 220, 221, 222, 223, 224, 225, 226, 227, 228, 229, 230, 231, 232, 233, 234, 235, 236, 237, 238, 239, 240, 241, 242, 243, 244, 245, 246, 247, 248, 249, 250, 300, 400, 500, 550, 1000 or more amino acids (or any derivable range therein).

A linker sequence may be included in the cytokine-peptide construction. For example, a linker having at least, at most, or exactly 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100 or more amino acids (or any derivable range therein) may separate that antibody and the peptide.

The ECM-affinity peptides of the disclosure may have affinity to one or more components of the extracellular matrix such as fibronectin, collagen, (collagen type I, collagen type III, and/or collagen type IV) tenascin C, fibrinogen, and fibrin. In certain aspects the ECM-affintiy peptide has an affinity for collagen. And in other asepcts the ECM-affinity peptide does not bind fibronectin.

### III. NUCLEIC ACIDS

In certain embodiments, the current disclosure concerns recombinant polynucleotides encoding the proteins, polypeptides, and peptides of the invention, such as ECM-affinity peptide operatively linked to cytokines or chemokines. Therefore, certain embodiments relate to nucleotides encoding for an ECM-affinity polypeptide and/or an ECM-affinity polypeptide or fragment thereof fused to a cytokine or fragment thereof.

As used in this application, the term “polynucleotide” refers to a nucleic acid molecule that either is recombinant or has been isolated free of total genomic nucleic acid. Included within the term “polynucleotide” are oligonucleotides (nucleic acids of 100 residues or less in length), recombinant vectors, including, for example, plasmids, cosmids, phage, viruses, and the like. Polynucleotides include, in certain aspects, regulatory sequences, isolated substantially away from their naturally occurring genes or protein encoding sequences. Polynucleotides may be single-stranded (coding or antisense) or double-stranded, and may be RNA, DNA (genomic, cDNA or synthetic), analogs thereof, or a combination thereof. Additional coding or non-coding sequences may, but need not, be present within a polynucleotide.

In this respect, the term “gene,” “polynucleotide,” or “nucleic acid” is used to refer to a nucleic acid that encodes a protein, polypeptide, or peptide (including any sequences required for proper transcription, post-translational modification, or localization). As will be understood by those in the art, this term encompasses genomic sequences, expression cassettes, cDNA sequences, and smaller engineered nucleic acid segments that express, or may be adapted to express, proteins, polypeptides, domains, peptides, fusion proteins, and mutants. A nucleic acid encoding all or part of a polypeptide may contain a contiguous nucleic acid sequence of: 10, 20, 30, 40, 50, 60, 70, 80, 90, 100, 110, 120, 130, 140, 150, 160, 170, 180, 190, 200, 210, 220, 230, 240, 250, 260, 270, 280, 290, 300, 310, 320, 330, 340, 350, 360, 370, 380, 390, 400, 410, 420, 430, 440, 441, 450, 460, 470, 480, 490, 500, 510, 520, 530, 540, 550, 560, 570, 580, 590, 600, 610, 620, 630, 640, 650, 660, 670, 680, 690, 700, 710, 720, 730, 740, 750, 760, 770, 780, 790, 800, 810, 820, 830, 840, 850, 860, 870, 880, 890, 900, 910, 920, 930, 940, 950, 960, 970, 980, 990, 1000, 1010, 1020, 1030, 1040, 1050, 1060, 1070, 1080, 1090, 1095, 1100, 1500, 2000, 2500, 3000, 3500, 4000, 4500, 5000, 5500, 6000, 6500, 7000, 7500, 8000, 9000, 10000, or more nucleotides, nucleosides, or base pairs, including all values and ranges there between, of a polynucleotide encoding one or more amino acid sequence described or referenced herein. It also is contemplated that a particular polypeptide may be encoded by nucleic acids containing variations having slightly different nucleic acid sequences but, nonetheless, encode the same or substantially similar protein.

In particular embodiments, the invention concerns isolated nucleic acid segments and recombinant vectors incorporating nucleic acid sequences that encode a polypeptide or peptide of the disclosure. The term “recombinant” may be used in conjunction with a polynucleotide or polypeptide and generally refers to a polypeptide or polynucleotide produced and/or manipulated in vitro or that is a replication product of such a molecule.

In other embodiments, the invention concerns isolated nucleic acid segments and recombinant vectors incorporating nucleic acid sequences that encode a polypeptide or peptide of the disclosure.

The nucleic acid segments used in the current disclosure can be combined with other nucleic acid sequences, such as promoters, polyadenylation signals, additional restriction enzyme sites, multiple cloning sites, other coding segments, and the like, such that their overall length may vary considerably. It is therefore contemplated that a nucleic acid fragment of almost any length may be employed, with the total length preferably being limited by the ease of preparation and use in the intended recombinant nucleic acid protocol. In some cases, a nucleic acid sequence may encode a polypeptide sequence with additional heterologous coding sequences, for example to allow for purification of the polypeptide, transport, secretion, post-translational modification, or for therapeutic benefits such as targeting or efficacy. As discussed above, a tag or other heterologous polypeptide may be added to the modified polypeptide-encoding sequence, wherein “heterologous” refers to a polypeptide that is not the same as the modified polypeptide.

In certain embodiments, the current disclosure provides polynucleotide variants having substantial identity to the sequences disclosed herein; those comprising at least 70%, 75%, 80%, 85%, 90%, 95%, 96%, 97%, 98%, or 99% or higher sequence identity, including all values and ranges there between, compared to a polynucleotide sequence of this disclosure using the methods described herein (e.g., BLAST analysis using standard parameters).

The disclosure also contemplates the use of polynucleotides which are complementary to all the above described polynucleotides.

### 1. Vectors

Polypeptides of the disclosure may be encoded by a nucleic acid molecule comprised in a vector. The term “vector” is used to refer to a carrier nucleic acid molecule into which a heterologous nucleic acid sequence can be inserted for introduction into a cell where it can be replicated and expressed. A nucleic acid sequence can be “heterologous,” which means that it is in a context foreign to the cell in which the vector is being introduced or to the nucleic acid in which is incorporated, which includes a sequence homologous to a sequence in the cell or nucleic acid but in a position within the host cell or nucleic acid where it is ordinarily not found. Vectors include DNAs, RNAs, plasmids, cosmids, viruses (bacteriophage, animal viruses, and plant viruses), and artificial chromosomes (e.g., YACs). One of skill in the art would be well equipped to construct a vector through standard recombinant techniques (for example Sambrook et al., 2001; Ausubel et al., 1996, both incorporated herein by reference). In addition to encoding a polypeptide of the disclosure, the vector can encode other polypeptide sequences such as a one or more other bacterial peptide, a tag, or an immunogenicity enhancing peptide. Useful vectors encoding such fusion proteins include pIN vectors (Inouye et al., 1985), vectors encoding a stretch of histidines, and pGEX vectors, for use in generating glutathione S-transferase (GST) soluble fusion proteins for later purification and separation or cleavage.

The term “expression vector” refers to a vector containing a nucleic acid sequence coding for at least part of a gene product capable of being transcribed. In some cases, RNA molecules are then translated into a protein, polypeptide, or peptide. Expression vectors can contain a variety of “control sequences,” which refer to nucleic acid sequences necessary for the transcription and possibly translation of an operably linked coding sequence in a particular host organism. In addition to control sequences that govern transcription and translation, vectors and expression vectors may contain nucleic acid sequences that serve other functions as well and are described herein.

### 2. Promoters and Enhancers

A “promoter” is a control sequence. The promoter is typically a region of a nucleic acid sequence at which initiation and rate of transcription are controlled. It may contain genetic elements at which regulatory proteins and molecules may bind such as RNA polymerase and other transcription factors. The phrases “operatively positioned,” “operatively linked,” “under control,” and “under transcriptional control” mean that a promoter is in a correct functional location and/or orientation in relation to a nucleic acid sequence to control transcriptional initiation and expression of that sequence. A promoter may or may not be used in conjunction with an “enhancer,” which refers to a cis-acting regulatory sequence involved in the transcriptional activation of a nucleic acid sequence.

Naturally, it may be important to employ a promoter and/or enhancer that effectively directs the expression of the DNA segment in the cell type or organism chosen for expression. Those of skill in the art of molecular biology generally know the use of promoters, enhancers, and cell type combinations for protein expression (see Sambrook et al., 2001, incorporated herein by reference). The promoters employed may be constitutive, tissue-specific, or inducible and in certain embodiments may direct high level expression of the introduced DNA segment under specified conditions, such as large-scale production of recombinant proteins or peptides.

Various elements/promoters may be employed in the context of the present invention to regulate the expression of a gene. Examples of such inducible elements, which are regions of a nucleic acid sequence that can be activated in response to a specific stimulus, include but are not limited to Immunoglobulin Heavy Chain (Banerji et al., 1983; Gilles et al., 1983; Grosschedl et al., 1985; Atchinson et al., 1986, 1987; Imler et al., 1987; Weinberger et al., 1984; Kiledjian et al., 1988; Porton et al.; 1990), Immunoglobulin Light Chain (Queen et al., 1983; Picard et al., 1984), T Cell Receptor (Luria et al., 1987; Winoto et al., 1989; Redondo et al.; 1990), HLA DQ α and/or DQ β (Sullivan et al., 1987), β Interferon (Goodbourn et al., 1986; Fujita et al., 1987; Goodbourn et al., 1988), Interleukin-2 (Greene et al., 1989), Interleukin-2 Receptor (Greene et al., 1989; Lin et al., 1990), MHC Class II 5 (Koch et al., 1989), MHC Class II HLA-DRα (Sherman et al., 1989), β-Actin (Kawamoto et al., 1988; Ng et al.; 1989), Muscle Creatine Kinase (MCK) (Jaynes et al., 1988; Horlick et al., 1989; Johnson et al., 1989), Prealbumin (Transthyretin) (Costa et al., 1988), Elastase I (Ornitz et al., 1987), Metallothionein (MTII) (Karin et al., 1987; Culotta et al., 1989), Collagenase (Pinkert et al., 1987; Angel et al., 1987), Albumin (Pinkert et al., 1987; Tronche et al., 1989, 1990), α-Fetoprotein (Godbout et al., 1988; Campere et al., 1989), γ-Globin (Bodine et al., 1987; Perez-Stable et al., 1990), β-Globin (Trudel et al., 1987), c-fos (Cohen et al., 1987), c-Ha-Ras (Triesman, 1986; Deschamps et al., 1985), Insulin (Edlund et al., 1985), Neural Cell Adhesion Molecule (NCAM) (Hirsh et al., 1990), α1-Antitrypain (Latimer et al., 1990), H2B (TH2B) Histone (Hwang et al., 1990), Mouse and/or Type I Collagen (Ripe et al., 1989), Glucose-Regulated Proteins (GRP94 and GRP78) (Chang et al., 1989), Rat Growth Hormone (Larsen et al., 1986), Human Serum Amyloid A (SAA) (Edbrooke et al., 1989), Troponin I (TN I) (Yutzey et al., 1989), Platelet-Derived Growth Factor (PDGF) (Pech et al., 1989), Duchenne Muscular Dystrophy (Klamut et al., 1990), SV40 (Banerji et al., 1981; Moreau et al., 1981; Sleigh et al., 1985; Firak et al., 1986; Herr et al., 1986; Imbra et al., 1986; Kadesch et al., 1986; Wang et al., 1986; Ondek et al., 1987; Kuhl et al., 1987; Schaffner et al., 1988), Polyoma (Swartzendruber et al., 1975; Vasseur et al., 1980; Katinka et al., 1980, 1981; Tyndell et al., 1981; Dandolo et al., 1983; de Villiers et al., 1984; Hen et al., 1986; Satake et al., 1988; Campbell et al., 1988), Retroviruses (Kriegler et al., 1982, 1983; Levinson et al., 1982; Kriegler et al., 1983, 1984a, b, 1988; Bosze et al., 1986; Miksicek et al., 1986; Celander et al., 1987; Thiesen et al., 1988; Celander et al., 1988; Choi et al., 1988; Reisman et al., 1989), Papilloma Virus (Campo et al., 1983; Lusky et al., 1983; Spandidos and Wilkie, 1983; Spalholz et al., 1985; Lusky et al., 1986; Cripe et al., 1987; Gloss et al., 1987; Hirochika et al., 1987; Stephens et al., 1987), Hepatitis B Virus (Bulla et al., 1986; Jameel et al., 1986; Shaul et al., 1987; Spandau et al., 1988; Vannice et al., 1988), Human Immunodeficiency Virus (Muesing et al., 1987; Hauber et al., 1988; Jakobovits et al., 1988; Feng et al., 1988; Takebe et al., 1988; Rosen et al., 1988; Berkhout et al., 1989; Laspia et al., 1989; Sharp et al., 1989; Braddock et al., 1989), Cytomegalovirus (CMV) IE (Weber et al., 1984; Boshart et al., 1985; Foecking et al., 1986), Gibbon Ape Leukemia Virus (Holbrook et al., 1987; Quinn et al., 1989).

Inducible elements include, but are not limited to MT II—Phorbol Ester (TFA)/Heavy metals (Palmiter et al., 1982; Haslinger et al., 1985; Searle et al., 1985; Stuart et al., 1985; Imagawa et al., 1987, Karin et al., 1987; Angel et al., 1987b; McNeall et al., 1989); MMTV (mouse mammary tumor virus)—Glucocorticoids (Huang et al., 1981; Lee et al., 1981; Majors et al., 1983; Chandler et al., 1983; Lee et al., 1984; Ponta et al., 1985; Sakai et al., 1988); β-Interferon—poly(rI)x/poly(rc) (Tavernier et al., 1983); Adenovirus 5 E2—EIA (Imperiale et al., 1984); Collagenase—Phorbol Ester (TPA) (Angel et al., 1987a); Stromelysin—Phorbol Ester (TPA) (Angel et al., 1987b); SV40—Phorbol Ester (TPA) (Angel et al., 1987b); Murine MX Gene—Interferon, Newcastle Disease Virus (Hug et al., 1988); GRP78 Gene—A23187 (Resendez et al., 1988); α-2-Macroglobulin—IL-6 (Kunz et al., 1989); Vimentin—Serum (Rittling et al., 1989); MHC Class I Gene H-2κb—Interferon (Blanar et al., 1989); HSP70—ElA/SV40 Large T Antigen (Taylor et al., 1989, 1990a, 1990b); Proliferin—Phorbol Ester/TPA (Mordacq et al., 1989); Tumor Necrosis Factor—PMA (Hensel et al., 1989); and Thyroid Stimulating Hormone α Gene—Thyroid Hormone (Chatterjee et al., 1989).

The particular promoter that is employed to control the expression of peptide or protein encoding polynucleotide of the invention is not believed to be critical, so long as it is capable of expressing the polynucleotide in a targeted cell, preferably a bacterial cell. Where a human cell is targeted, it is preferable to position the polynucleotide coding region adjacent to and under the control of a promoter that is capable of being expressed in a human cell. Generally speaking, such a promoter might include either a bacterial, human or viral promoter.

### 3. Initiation Signals and Internal Ribosome Binding Sites (IRES)

A specific initiation signal also may be required for efficient translation of coding sequences. These signals include the ATG initiation codon or adjacent sequences. Exogenous translational control signals, including the ATG initiation codon, may need to be provided. One of ordinary skill in the art would readily be capable of determining this and providing the necessary signals.

In certain embodiments of the invention, the use of internal ribosome entry sites (IRES) elements are used to create multigene, or polycistronic, messages. IRES elements are able to bypass the ribosome scanning model of 5′ methylated Cap dependent translation and begin translation at internal sites (Pelletier and Sonenberg, 1988; Macejak and Sarnow, 1991). IRES elements can be linked to heterologous open reading frames. Multiple open reading frames can be transcribed together, each separated by an IRES, creating polycistronic messages. Multiple genes can be efficiently expressed using a single promoter/enhancer to transcribe a single message (see U.S. Pat. Nos. 5,925,565 and 5,935,819, herein incorporated by reference).

### 4. Selectable and Screenable Markers

In certain embodiments of the invention, cells containing a nucleic acid construct of the current disclosure may be identified in vitro or in vivo by encoding a screenable or selectable marker in the expression vector. When transcribed and translated, a marker confers an identifiable change to the cell permitting easy identification of cells containing the expression vector. Generally, a selectable marker is one that confers a property that allows for selection. A positive selectable marker is one in which the presence of the marker allows for its selection, while a negative selectable marker is one in which its presence prevents its selection. An example of a positive selectable marker is a drug resistance marker.

### 5. Host Cells

As used herein, the terms “cell,” “cell line,” and “cell culture” may be used interchangeably. All of these terms also include their progeny, which is any and all subsequent generations. It is understood that all progeny may not be identical due to deliberate or inadvertent mutations. In the context of expressing a heterologous nucleic acid sequence, “host cell” refers to a prokaryotic or eukaryotic cell, and it includes any transformable organism that is capable of replicating a vector or expressing a heterologous gene encoded by a vector. A host cell can, and has been, used as a recipient for vectors or viruses. A host cell may be “transfected” or “transformed,” which refers to a process by which exogenous nucleic acid, such as a recombinant protein-encoding sequence, is transferred or introduced into the host cell. A transformed cell includes the primary subject cell and its progeny.

Host cells may be derived from prokaryotes or eukaryotes, including bacteria, yeast cells, insect cells, and mammalian cells for replication of the vector or expression of part or all of the nucleic acid sequence(s). Numerous cell lines and cultures are available for use as a host cell, and they can be obtained through the American Type Culture Collection (ATCC), which is an organization that serves as an archive for living cultures and genetic materials (www.atcc.org).

### 6. Expression Systems

Numerous expression systems exist that comprise at least a part or all of the compositions discussed above. Prokaryote- and/or eukaryote-based systems can be employed for use with the present invention to produce nucleic acid sequences, or their cognate polypeptides, proteins and peptides. Many such systems are commercially and widely available.

The insect cell/baculovirus system can produce a high level of protein expression of a heterologous nucleic acid segment, such as described in U.S. Pat. Nos. 5,871,986, 4,879,236, both herein incorporated by reference, and which can be bought, for example, under the name MAXBAC® 2.0 from INVITROGEN® and BACPACK™ BACULOVIRUS EXPRESSION SYSTEM FROM CLONTECH®.

In addition to the disclosed expression systems of the invention, other examples of expression systems include STRATAGENE®'s COMPLETE CONTROL™ Inducible Mammalian Expression System, which involves a synthetic ecdysone-inducible receptor, or its pET Expression System, an E. coli expression system. Another example of an inducible expression system is available from INVITROGEN®, which carries the T-REX™ (tetracycline-regulated expression) System, an inducible mammalian expression system that uses the full-length CMV promoter. INVITROGEN® also provides a yeast expression system called the Pichia methanolica Expression System, which is designed for high-level production of recombinant proteins in the methylotrophic yeast Pichia methanolica. One of skill in the art would know how to express a vector, such as an expression construct, to produce a nucleic acid sequence or its cognate polypeptide, protein, or peptide.

### IV. COMBINATION THERAPY

The compositions and related methods of the present disclosure, particularly administration of and ECM-affinity peptide operatively linked to a cytokine may also be used in combination with the administration of additional therapies such as the additional therapeutics described herein or in combination with other traditional therapeutics known in the art.

The therapeutic compositions and treatments disclosed herein may precede, be co-current with and/or follow another treatment or agent by intervals ranging from minutes to weeks. In embodiments where agents are applied separately to a cell, tissue or organism, one would generally ensure that a significant period of time did not expire between the time of each delivery, such that the therapeutic agents would still be able to exert an advantageously combined effect on the cell, tissue or organism. For example, in such instances, it is contemplated that one may contact the cell, tissue or organism with two, three, four or more agents or treatments substantially simultaneously (i.e., within less than about a minute). In other aspects, one or more therapeutic agents or treatments may be administered or provided within 1 minute, 5 minutes, 10 minutes, 20 minutes, 30 minutes, 45 minutes, 60 minutes, 2 hours, 3 hours, 4 hours, 5 hours, 6 hours, 7 hours, 8 hours, 9 hours, 10 hours, 11 hours, 12 hours, 13 hours, 14 hours, 15 hours, 16 hours, 17 hours, 18 hours, 19 hours, 20 hours, 21 hours, 22 hours, 22 hours, 23 hours, 24 hours, 25 hours, 26 hours, 27 hours, 28 hours, 29 hours, 30 hours, 31 hours, 32 hours, 33 hours, 34 hours, 35 hours, 36 hours, 37 hours, 38 hours, 39 hours, 40 hours, 41 hours, 42 hours, 43 hours, 44 hours, 45 hours, 46 hours, 47 hours, 48 hours, 1 day, 2 days, 3 days, 4 days, 5 days, 6 days, 7 days, 8 days, 9 days, 10 days, 11 days, 12 days, 13 days, 14 days, 15 days, 16 days, 17 days, 18 days, 19 days, 20 days, 21 days, 1 week, 2 weeks, 3 weeks, 4 weeks, 5 weeks, 6 weeks, 7 weeks, or 8 weeks or more, and any range derivable therein, prior to and/or after administering another therapeutic agent or treatment.

Various combination regimens of the therapeutic agents and treatments may be employed. Non-limiting examples of such combinations are shown below, wherein a therapeutic agent such as a composition disclosed herein is “A” and a second agent, such as an additional agent, chemotherapeutic, or checkpoint inhibitor described herein or known in the art is “B”:

| | | | | | | | |
|---|---|---|---|---|---|---|---|
| A/B/A | B/A/B | B/B/A | A/A/B | A/B/B | B/A/A | A/B/B/B | B/A/B/B |

| | | | | | |
|---|---|---|---|---|---|
| B/B/B/A | B/B/A/B | A/A/B/B | A/B/A/B | A/B/B/A | B/B/A/A |
| B/A/B/A | B/A/A/B | A/A/A/B | B/A/A/A | A/B/A/A | A/A/B/A |

In some embodiments, more than one course of therapy may be employed. It is contemplated that multiple courses may be implemented.

### 1. Chemotherapeutics

The term “chemotherapeutic agent,” refers to a therapeutic compound and/or drug which may be used to, among other things, treat cancer. For example, a chemotherapeutic agent may include, but is not limited to, any agent that interferes with cell division, disrupts normal functionality of microtubules, inhibits utilization of a metabolite, substitutes nucleotide analogs into cellular DNA, or inhibits enzymes necessary for DNA replication.

Suitable classes of chemotherapeutic agents include (a) Alkylating Agents, such as nitrogen mustards (e.g., mechlorethamine, cylophosphamide, ifosfamide, melphalan, chlorambucil), ethylenimines and methylmelamines (e.g., hexamethylmelamine, thiotepa), alkyl sulfonates (e.g., busulfan), nitrosoureas (e.g., carmustine, lomustine, chlorozoticin, streptozocin) and triazines (e.g., dicarbazine), (b) Antimetabolites, such as folic acid analogs (e.g., methotrexate), pyrimidine analogs (e.g., 5-fluorouracil, floxuridine, cytarabine, azauridine) and purine analogs and related materials (e.g., 6-mercaptopurine, 6-thioguanine, pentostatin), (c) Natural Products, such as vinca alkaloids (e.g., vinblastine, vincristine), epipodophylotoxins (e.g., etoposide, teniposide), antibiotics (e.g., dactinomycin, daunorubicin, doxorubicin, bleomycin, plicamycin and mitoxanthrone), enzymes (e.g., L-asparaginase), and biological response modifiers (e.g., Interferon-α), and (d) Miscellaneous Agents, such as platinum coordination complexes (e.g., cisplatin, carboplatin), substituted ureas (e.g., hydroxyurea), methylhydiazine derivatives (e.g., procarbazine), and adreocortical suppressants (e.g., taxol and mitotane). In some embodiments, cisplatin is a particularly suitable chemotherapeutic agent.

Other suitable chemotherapeutic agents include antimicrotubule agents, e.g., Paclitaxel (“Taxol”) and doxorubicin hydrochloride (“doxorubicin”).

Nitrogen mustards are another suitable chemotherapeutic agent useful in the methods of the disclosure. A nitrogen mustard may include, but is not limited to, mechlorethamine (HN₂), cyclophosphamide and/or ifosfamide, melphalan (L-sarcolysin), and chlorambucil. Cyclophosphamide (CYTOXAN®) is available from Mead Johnson and NEOSTAR® is available from Adria), is another suitable chemotherapeutic agent. Suitable oral doses for adults include, for example, about 1 mg/kg/day to about 5 mg/kg/day, intravenous doses include, for example, initially about 40 mg/kg to about 50 mg/kg in divided doses over a period of about 2 days to about 5 days or about 10 mg/kg to about 15 mg/kg about every 7 days to about 10 days or about 3 mg/kg to about 5 mg/kg twice a week or about 1.5 mg/kg/day to about 3 mg/kg/day. Because of adverse gastrointestinal effects, the intravenous route is preferred. The drug also sometimes is administered intramuscularly, by infiltration or into body cavities.

Additional suitable chemotherapeutic agents include pyrimidine analogs, such as cytarabine (cytosine arabinoside), 5-fluorouracil (fluouracil; 5-FU) and floxuridine (fluorode-oxyuridine; FudR). 5-FU may be administered to a subject in a dosage of anywhere between about 7.5 to about 1000 mg/m2. Further, 5-FU dosing schedules may be for a variety of time periods, for example up to six weeks, or as determined by one of ordinary skill in the art to which this disclosure pertains.

Gemcitabine diphosphate (GEMZAR®, Eli Lilly & Co., “gemcitabine”), another suitable chemotherapeutic agent, is recommended for treatment of advanced and metastatic pancreatic cancer, and will therefore be useful in the present disclosure for these cancers as well.

The amount of the chemotherapeutic agent delivered to the patient may be variable. In one suitable embodiment, the chemotherapeutic agent may be administered in an amount effective to cause arrest or regression of the cancer in a host, when the chemotherapy is administered with the construct. In other embodiments, the chemotherapeutic agent may be administered in an amount that is anywhere between 2 to 10,000 fold less than the chemotherapeutic effective dose of the chemotherapeutic agent. For example, the chemotherapeutic agent may be administered in an amount that is about 20 fold less, about 500 fold less or even about 5000 fold less than the chemotherapeutic effective dose of the chemotherapeutic agent. The chemotherapeutics of the disclosure can be tested in vivo for the desired therapeutic activity in combination with the construct, as well as for determination of effective dosages. For example, such compounds can be tested in suitable animal model systems prior to testing in humans, including, but not limited to, rats, mice, chicken, cows, monkeys, rabbits, etc. In vitro testing may also be used to determine suitable combinations and dosages, as described in the examples.

Actual dosage levels of the active ingredients in the methods of this disclosure may be varied so as to obtain an amount of the active ingredient which is effective to achieve the desired therapeutic response for a particular patient, composition and mode of administration, without being toxic to the patient. The selected dosage level will depend upon a variety of factors, including the activity of the chemotherapeutic agent selected, the route of administration, the time of administration, the rate of excretion of the chemotherapeutic agent, the duration of the treatment, other drugs, compounds and/or materials used in combination with the particular chemotherapeutic agent, the age, sex, weight, condition, general health and prior medical history of the patient being treated, and like factors well known in the medical arts.

It is envisioned that combining the effects of chemotherapy and the expression of the therapeutic polypeptide may enhance the antitumor effect of each of these agents if used alone (i.e., if the therapeutic polypeptide is administered directly, and not induced by the presence of the chemotherapeutic agent). A physician having ordinary skill in the art can readily determine and prescribe the effective amount of the construct and the chemotherapeutic agent required. For example, the physician could start doses of the construct and/or chemotherapy at levels lower than that required in order to achieve the desired therapeutic effect and gradually increase the dosage until the desired effect is achieved.

### 2. Ionizing Radiation

As used herein, “ionizing radiation” means radiation comprising particles or photons that have sufficient energy or can produce sufficient energy via nuclear interactions to produce ionization (gain or loss of electrons). An exemplary and preferred ionizing radiation is an x-radiation. Means for delivering x-radiation to a target tissue or cell are well known in the art.

In some embodiments, the amount of ionizing radiation is greater than 20 Gray and is administered in one dose. In some embodiments, the amount of ionizing radiation is 18 Gy and is administered in three doses. In some embodiments, the amount of ionizing radiation is at least, at most, or exactly 2, 4, 6, 8, 10, 15, 16, 17, 18, 19, 20, 21, 22, 23, 24, 25, 26, 27, 18, 19, 30, 31, 32, 33, 34, 35, 36, 37, 38, 39, 40, 41, 42, 43, 44, 45, 46, 47, 48, 49, or 40 Gy (or any derivable range therein). In some embodiments, the ionizing radiation is administered in at least, at most, or exactly 1, 2, 3, 4, 5, 6, 7, 8, 9, or 10 does (or any derivable range therein). When more than one dose is administered, the does may be about 1, 4, 8, 12, or 24 hours or 1, 2, 3, 4, 5, 6, 7, or 8 days or 1, 2, 3, 4, 5, 6, 7, 8, 9, 10, 12, 14, or 16 weeks apart, or any derivable range therein.

In some embodiments, the amount of IR may be presented as a total dose of IR, which is then administered in fractionated doses. For example, in some embodiments, the total dose is 50 Gy administered in 10 fractionated doses of 5 Gy each. In some embodiments, the total dose is 50-90 Gy, administered in 20-60 fractionated doses of 2-3 Gy each. In some embodiments, the total dose of IR is at least, at most, or about 20, 21, 22, 23, 24, 25, 26, 27, 28, 29, 30, 31, 32, 33, 34, 35, 36, 37, 38, 39, 40,41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 125, 130, 135, 140, or 150 (or any derivable range therein). In some embodiments, the total dose is administered in fractionated doses of at least, at most, or exactly 1, 2, 3, 4, 5, 6, 7, 8, 9, 10, 12, 14, 15, 20, 25, 30, 35, 40, 45, or 50 Gy (or any derivable range therein). In some embodiments, at least, at most, or exactly 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40,41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100 fractionated doses are administered (or any derivable range therein). In some embodiments, at least, at most, or exactly 1, 2, 3, 4, 5, 6, 7, 8, 9, 10, 11, or 12 (or any derivable range therein) fractionated doses are administered per day. In some embodiments, at least, at most, or exactly 1, 2, 3, 4, 5, 6, 7, 8, 9, 10, 11, 12, 13, 14, 15, 16, 17, 18, 19, 20, 21, 22, 23, 24, 25, 26, 27, 28, 29, or 30 (or any derivable range therein) fractionated doses are administered per week.

In some embodiments, an IR regimen and/or total IR dose is prescribed by a doctor or attending medical professional. The medical professional may monitor and/or access the progress of the patient throughout the administration of the IR and/or the medical professional may access the patient at the completion of the administration of the prescribed IR dose and prescribe a new dose/regimen of IR based on the assessment.

### 3. Additional Agents

In some embodiments, the method further comprises administration of an additional agent. In some embodiments, the additional agent is an immunostimulator. The term “immunostimulator” as used herein refers to a compound that can stimulate an immune response in a subject, and may include an adjuvant. In some embodiments, an immunostimulator is an agent that does not constitute a specific antigen, but can boost the strength and longevity of an immune response to an antigen. Such immunostimulators may include, but are not limited to stimulators of pattern recognition receptors, such as Toll-like receptors, RIG-1 and NOD-like receptors (NLR), mineral salts, such as alum, alum combined with monphosphoryl lipid (MPL) A of Enterobacteria, such as Escherihia coli, Salmonella minnesota, Salmonella typhimurium, or Shigella flexneri or specifically with MPL.RTM. (ASO4), MPL A of above-mentioned bacteria separately, saponins, such as QS-21, Quil-A, ISCOMs, ISCOMATRIX, emulsions such as MF59, Montanide, ISA 51and ISA 720, AS02 (QS21+squalene+MPL.), liposomes and liposomal formulations such as AS01, synthesized or specifically prepared microparticles and microcarriers such as bacteria-derived outer membrane vesicles (OMV) of N. gonorrheae, Chlamydia trachomatis and others, or chitosan particles, depot-forming agents, such as Pluronic block co-polymers, specifically modified or prepared peptides, such as muramyl dipeptide, aminoalkyl glucosaminide 4-phosphates, such as RC529, or proteins, such as bacterial toxoids or toxin fragments.

In some embodiments, the additional agent comprises an agonist for pattern recognition receptors (PRR), including, but not limited to Toll-Like Receptors (TLRs), specifically TLRs 2, 3, 4, 5, 7, 8, 9 and/or combinations thereof. In some embodiments, additional agents comprise agonists for Toll-Like Receptors 3, agonists for Toll-Like Receptors 7 and 8, or agonists for Toll-Like Receptor 9; preferably the recited immunostimulators comprise imidazoquinolines; such as R848; adenine derivatives, such as those disclosed in U.S. Pat. No. 6,329,381, U.S. Published Patent Application 2010/0075995, or WO 2010/018132; immunostimulatory DNA; or immunostimulatory RNA. In some embodiments, the additional agents also may comprise immunostimulatory RNA molecules, such as but not limited to dsRNA, poly I:C or poly I:poly C12U (available as Ampligen. RTM., both poly I:C and poly I:polyC12U being known as TLR3 stimulants), and/or those disclosed in F. Heil et al., “Species-Specific Recognition of Single-Stranded RNA via Toll-like Receptor 7 and 8” Science 303(5663), 1526-1529 (2004); J. Vollmer et al., “Immune modulation by chemically modified ribonucleosides and oligoribonucleotides” WO 2008033432 A2; A. Forsbach et al., “Immunostimulatory oligoribonucleotides containing specific sequence motif(s) and targeting the Toll-like receptor 8 pathway” WO 2007062107 A2; E. Uhlmann et al., “Modified oligoribonucleotide analogs with enhanced immunostimulatory activity” U.S. Pat. Appl. Publ. US 2006241076; G. Lipford et al., “Immunostimulatory viral RNA oligonucleotides and use for treating cancer and infections” WO 2005097993 A2; G. Lipford et al., “Immunostimulatory G,U-containing oligoribonucleotides, compositions, and screening methods” WO 2003086280 A2. In some embodiments, an additional agent may be a TLR-4 agonist, such as bacterial lipopolysaccharide (LPS), VSV-G, and/or HMGB-1. In some embodiments, additional agents may comprise TLR-5 agonists, such as flagellin, or portions or derivatives thereof, including but not limited to those disclosed in U.S. Pat. Nos. 6,130,082, 6,585,980, and 7,192,725.

In some embodiments, additional agents may be proinflammatory stimuli released from necrotic cells (e.g., urate crystals). In some embodiments, additional agents may be activated components of the complement cascade (e.g., CD21, CD35, etc.). In some embodiments, additional agents may be activated components of immune complexes. Additional agents also include complement receptor agonists, such as a molecule that binds to CD21 or CD35. In some embodiments, the complement receptor agonist induces endogenous complement opsonization of the synthetic nanocarrier. In some embodiments, immunostimulators are cytokines, which are small proteins or biological factors (in the range of 5 kDa-20 kDa) that are released by cells and have specific effects on cell-cell interaction, communication and behavior of other cells. In some embodiments, the cytokine receptor agonist is a small molecule, antibody, fusion protein, or aptamer.

In some embodiments, the additional agent is an antibody-drug conjugate. In some embodiments, the antibody-drug conjugate is selected from gemtuzumab ozogamicin, brentuximab vedotin, and trastuzumab emtansine.

In some embodiments, the additional agent is a chimeric antigen receptor (CAR). CARs are artificial T cell receptors which graft a specificity onto an immune effector cell. The most common form of these molecules are fusions of single-chain variable fragments (scFv) derived from monoclonal antibodies, fused to CD3-zeta transmembrane and endodomain. Such molecules result in the transmission of a zeta signal in response to recognition by the scFv of its target. An example of such a construct is 14g2a-Zeta, which is a fusion of a scFv derived from hybridoma 14g2a (which recognizes disialoganglioside GD2). When T cells express this molecule (usually achieved by oncoretroviral vector transduction), they recognize and kill target cells that express GD2 (e.g., neuroblastoma cells). The variable portions of an immunoglobulin heavy and light chain are fused by a flexible linker to form a scFv. This scFv is preceded by a signal peptide to direct the nascent protein to the endoplasmic reticulum and subsequent surface expression (this is cleaved). A flexible spacer allows the scFv to orient in different directions to enable antigen binding. The transmembrane domain is a typical hydrophobic alpha helix usually derived from the original molecule of the signalling endodomain which protrudes into the cell and transmits the desired signal.

### V. THERAPEUTIC METHODS

The current methods and compositions relate to methods for treating cancer. In some embodiments, the cancer comprises a solid tumor. In some embodiments, the cancer is non-lymphatic. In some embodiments, the cancer is melanoma or colon cancer.

The compositions of the disclosure may be used for in vivo, in vitro, or ex vivo administration. The route of administration of the composition may be, for example, intratumoral, intracutaneous, subcutaneous, intravenous, intralymphatic, and intraperitoneal administrations. In some embodiments, the administration is intravenous or intratumoral or intralymphatic or peri-tumoral. In some embodiments, the compositions are administered directly into a cancer tissue or a lymph node.

“Tumor,” as used herein, refers to all neoplastic cell growth and proliferation, whether malignant or benign, and all pre-cancerous and cancerous cells and tissues. The terms “cancer,” “cancerous,” “cell proliferative disorder,” “proliferative disorder,” and “tumor” are not mutually exclusive as referred to herein.

The cancers amenable for treatment include, but are not limited to, tumors of all types, locations, sizes, and characteristics. The methods and compositions of the disclosure are suitable for treating, for example, melanoma, colon cancer, pancreatic cancer, colon cancer, acute myeloid leukemia, adrenocortical carcinoma, AIDS-related cancers, AIDS-related lymphoma, anal cancer, appendix cancer, astrocytoma, childhood cerebellar or cerebral basal cell carcinoma, bile duct cancer, extrahepatic bladder cancer, bone cancer, osteosarcoma/malignant fibrous histiocytoma, brainstem glioma, brain cerebellar brain tumor, astrocytoma tumor, cerebral astrocytoma/malignant glioma brain tumor, ependymoma brain tumor, medulloblastoma brain tumor, supratentorial primitive neuroectodermal tumors brain tumor, visual pathway and hypothalamic glioma, breast cancer, specific breast cancers such as ductal carcinoma in situ, invasive ductal carcinoma, tubular carcinoma of the breast, medullary carcinoma of the breast, mucinous carcinoma of the breast, papillary carcinoma of the breast, cribriform carcinoma of the breast, invasive lobular carcinoma, inflammatory breast cancer, lobular carcinoma in situ, male breast cancer, paget's disease of the nipple, phyllodes tumors of the breast, recurrent and/or metastatic breast, cancer, luminal A or B breast cancer, triple-negative/basal-like breast cancer, and HER2-enriched breast cancer, lymphoid cancer, bronchial adenomas/carcinoids, tracheal cancer, Burkitt lymphoma, carcinoid tumor, childhood carcinoid tumor, gastrointestinal carcinoma of unknown primary, central nervous system lymphoma, primary cerebellar astrocytoma, childhood cerebral astrocytoma/malignant glioma, childhood cervical cancer, childhood cancers, chronic lymphocytic leukemia, chronic myelogenous leukemia, chronic myeloproliferative disorders, cutaneous T-cell lymphoma, desmoplastic small round cell tumor, endometrial cancer, ependymoma, esophageal cancer, Ewing's, childhood extragonadal Germ cell tumor, extrahepatic bile duct cancer, eye cancer, retinoblastoma, gallbladder cancer, gastric (stomach) cancer, gastrointestinal carcinoid tumor, gastrointestinal stromal tumor (GIST), germ cell tumor: extracranial, extragonadal, or ovarian, gestational trophoblastic tumor, glioma of the brain stem, glioma, childhood cerebral astrocytoma, childhood visual pathway and hypothalamic glioma, gastric carcinoid, hairy cell leukemia, head and neck cancer, heart cancer, hepatocellular (liver) cancer, Hodgkin lymphoma, hypopharyngeal cancer, hypothalamic and visual pathway glioma, childhood intraocular melanoma, islet cell carcinoma (endocrine pancreas), kaposi sarcoma, kidney cancer (renal cell cancer), laryngeal cancer, leukemia, acute lymphoblastic (also called acute lymphocytic leukemia) leukemia, acute myeloid (also called acute myelogenous leukemia) leukemia, chronic lymphocytic (also called chronic lymphocytic leukemia) leukemia, chronic myelogenous (also called chronic myeloid leukemia) leukemia, hairy cell lip and oral cavity cancer, liposarcoma, liver cancer (primary), non-small cell lung cancer, small cell lung cancer, lymphomas, AIDS-related lymphoma, Burkitt lymphoma, cutaneous T-cell lymphoma, Hodgkin lymphoma, Non-Hodgkin (an old classification of all lymphomas except Hodgkin's) lymphoma, primary central nervous system lymphoma, Waldenstrom macroglobulinemia, malignant fibrous histiocytoma of bone/osteosarcoma, childhood medulloblastoma, intraocular (eye) melanoma, merkel cell carcinoma, adult malignant mesothelioma, childhood mesothelioma, metastatic squamous neck cancer, mouth cancer, multiple endocrine neoplasia syndrome, multiple myeloma/plasma cell neoplasm, mycosis fungoides, myelodysplastic syndromes, myelodysplastic/myeloproliferative diseases, chronic myelogenous leukemia, adult acute myeloid leukemia, childhood acute myeloid leukemia, multiple myeloma, chronic myeloproliferative disorders, nasal cavity and paranasal sinus cancer, nasopharyngeal carcinoma, neuroblastoma, oral cancer, oropharyngeal cancer, osteosarcoma/malignant, fibrous histiocytoma of bone, ovarian cancer, ovarian epithelial cancer (surface epithelial-stromal tumor), ovarian germ cell tumor, ovarian low malignant potential tumor, pancreatic cancer, islet cell paranasal sinus and nasal cavity cancer, parathyroid cancer, penile cancer, pharyngeal cancer, pheochromocytoma, pineal astrocytoma, pineal germinoma, pineoblastoma and supratentorial primitive neuroectodermal tumors, childhood pituitary adenoma, plasma cell neoplasia/multiple myeloma, pleuropulmonary blastoma, primary central nervous system lymphoma, prostate cancer, rectal cancer, renal cell carcinoma (kidney cancer), renal pelvis and ureter transitional cell cancer, retinoblastoma, rhabdomyosarcoma, childhood Salivary gland cancer Sarcoma, Ewing family of tumors, Kaposi sarcoma, soft tissue sarcoma, uterine sezary syndrome sarcoma, skin cancer (nonmelanoma), skin cancer (melanoma), skin carcinoma, Merkel cell small cell lung cancer, small intestine cancer, soft tissue sarcoma, squamous cell carcinoma. squamous neck cancer with occult primary, metastatic stomach cancer, supratentorial primitive neuroectodermal tumor, childhood T-cell lymphoma, testicular cancer, throat cancer, thymoma, childhood thymoma, thymic carcinoma, thyroid cancer, urethral cancer, uterine cancer, endometrial uterine sarcoma, vaginal cancer, visual pathway and hypothalamic glioma, childhood vulvar cancer, and wilms tumor (kidney cancer).

### VI. PHARMACEUTICAL COMPOSITIONS AND METHODS

In some embodiments, pharmaceutical compositions are administered to a subject. Different aspects involve administering an effective amount of a composition to a subject. In some embodiments, a composition comprising an anti-cancer agent may be administered to the subject or patient to treat cancer or reduce the size of a tumor. Additionally, such compounds can be administered in combination with an additional cancer therapy.

Compositions can be formulated for parenteral administration, e.g., formulated for injection via the intravenous, transcatheter injection, intraarterial injection, intramuscular, sub-cutaneous, or even intraperitoneal routes. Typically, such compositions can be prepared as injectables, either as liquid solutions or suspensions; solid forms suitable for use to prepare solutions or suspensions upon the addition of a liquid prior to injection can also be prepared; and, the preparations can also be emulsified. The preparation of such formulations will be known to those of skill in the art in light of the present disclosure. Other routes of administration include intratumoral, peri-tumoral, intralymphatic, injection into cancer tissue, and injection into lymph nodes. In some embodiments, the administration is systemic.

Other routes of administration are also contemplated. For example, the constructs and agents may be administered in association with a carrier. In some embodiments, the carrier is a nanoparticle or microparticle. In some embodiments, the nanoparticle or microparticle is a tumor directed nanoparticle or microparticle. For example, the carrier may further comprise a targeting moiety that directs the carrier to the tumor. The targeting moiety may be a binding agent (e.g., antibody, including scFv, etc. or other antigen binding agent) that specifically recognizes tumor cells. In some embodiments, the construct is enclosed within the carrier. In some embodiments, the construct is covalently or non-covalently attached to the surface of the carrier. In some embodiments, the carrier is a liposome.

Particles can have a structure of variable dimension and known variously as a microsphere, microparticle, nanoparticle, nanosphere, or liposome. Such particulate formulations can be formed by covalent or non-covalent coupling of the construct to the particle.

By “particle,” “microparticle,” “bead,” “microsphere,” and grammatical equivalents herein is meant small discrete particles that are administrable to a subject. In certain embodiments, the particles are substantially spherical in shape. The term “substantially spherical,” as used herein, means that the shape of the particles does not deviate from a sphere by more than about 10%.

The particles typically consist of a substantially spherical core and optionally one or more layers. The core may vary in size and composition. In addition to the core, the particle may have one or more layers to provide functionalities appropriate for the applications of interest. The thicknesses of layers, if present, may vary depending on the needs of the specific applications. For example, layers may impart useful optical properties.

Layers may also impart chemical or biological functionalities, referred to herein as chemically active or biologically active layers, and for these functionalities the layer or layers may typically range in thickness from about 0.001 micrometers (1 nanometer) to about 10 micrometers or more (depending on the desired particle diameter), these layers typically being applied on the outer surface of the particle.

The compositions of the core and layers may vary. Suitable materials for the particles or the core include, but are not limited to polymers, ceramics, glasses, minerals, and the like. Examples include, but are not limited to, standard and specialty glasses, silica, polystyrene, polyester, polycarbonate, acrylic polymers, polyacrylamide, polyacrylonitrile, polyamide, fluoropolymers, silicone, celluloses, silicon, metals (e.g., iron, gold, silver), minerals (e.g., ruby), nanoparticles (e.g., gold nanoparticles, colloidal particles, metal oxides, metal sulfides, metal selenides, and magnetic materials such as iron oxide), and composites thereof. The core could be of homogeneous composition, or a composite of two or more classes of material depending on the properties desired. In certain aspects, metal nanoparticles will be used. These metal particles or nanoparticles can be formed from Au, Pt, Pd, Cu, Ag, Co, Fe, Ni, Mn, Sm, Nd, Pr, Gd, Ti, Zr, Si, and In, precursors, their binary alloys, their ternary alloys and their intermetallic compounds. See U.S. Pat. No. 6,712,997, which is incorporated herein by reference in its entirety.

As previously stated, the particle may, in addition to the core, include one or more layers. The purposes for including layers in the microparticle may vary. Alternatively, the surface of the particle may be functionalized directly. A layer may provide suitable surfaces for attaching chemical functionalities for chemical binding or coupling sites.

Layers can be produced on the microparticles in a variety of ways known to those skilled in the art. Examples include sol-gel chemistry techniques such as described in Iler (1979); Brinker and Scherer (1990). Additional approaches to producing layers on particles include surface chemistry and encapsulation techniques such as described in Partch and Brown (1998); Pekareket al. (1994); Hanprasopwattana (1996); Davies (1998); and references therein. Vapor deposition techniques may also be used; see for example Golman and Shinohara (2000); and U.S. Pat. No. 6,387,498. Still other approaches include layer-by-layer self-assembly techniques such as described in Sukhorukov et al. (1998); Caruso et al. (1998); Caruso et al. (1999); U.S. Pat. No. 6,103,379 and references cited therein.

Particles may be formed by contacting an aqueous phase containing the construct and a polymer and a nonaqueous phase followed by evaporation of the nonaqueous phase to cause the coalescence of particles from the aqueous phase as taught in U.S. Pat. Nos. 4,589,330 or 4,818,542. Preferred polymers for such preparations are natural or synthetic copolymers or polymers selected from the group consisting of gleatin agar, starch, arabinogalactan, albumin, collagen, polyglycolic acid, polylactic acid, glycolide-L(-)lactide poly(episilon-caprolactone, poly(epsilon-caprolactone-CO-lactic acid), poly(epsilon-caprolactone-CO-glycolic acid), poly(β-hydroxy butyric acid), polyethylene oxide, polyethylene, poly(alkyl-2-cyanoacrylate), poly(hydroxyethyl methacrylate), polyamides, poly(amino acids), poly(2-hydroxyethyl DL-aspartamide), poly(ester urea), poly(L-phenylalanine/ethylene glycol/1,6-diisocyanatohexane) and poly(methyl methacrylate). Particularly preferred polymers are polyesters, such as polyglycolic acid, polylactic acid, glycolide-L(-)lactide poly(episilon-caprolactone, poly(epsilon-caprolactone-CO-lactic acid), and poly(epsilon-caprolactone-CO-glycolic acid. Solvents useful for dissolving the polymer include: water, hexafluoroisopropanol, methylenechloride, tetrahydrofuran, hexane, benzene, or hexafluoroacetone sesquihydrate.

The pharmaceutical forms suitable for injectable use include sterile aqueous solutions or dispersions; formulations including sesame oil, peanut oil, or aqueous propylene glycol; and sterile powders for the extemporaneous preparation of sterile injectable solutions or dispersions. In all cases the form must be sterile and must be fluid to the extent that it may be easily injected. It also should be stable under the conditions of manufacture and storage and must be preserved against the contaminating action of microorganisms, such as bacteria and fungi.

The carrier also can be a solvent or dispersion medium containing, for example, water, ethanol, polyol (for example, glycerol, propylene glycol, and liquid polyethylene glycol, and the like), suitable mixtures thereof, and vegetable oils. The proper fluidity can be maintained, for example, by the use of a coating, such as lecithin, by the maintenance of the required particle size in the case of dispersion, and by the use of surfactants. The prevention of the action of microorganisms can be brought about by various antibacterial and antifungal agents, for example, parabens, chlorobutanol, phenol, sorbic acid, thimerosal, and the like. In many cases, it will be preferable to include isotonic agents, for example, sugars or sodium chloride. Prolonged absorption of the injectable compositions can be brought about by the use in the compositions of agents delaying absorption, for example, aluminum monostearate and gelatin.

Sterile injectable solutions are prepared by incorporating the active compounds in the required amount in the appropriate solvent with various of the other ingredients enumerated above, as required, followed by filtered sterilization. Generally, dispersions are prepared by incorporating the various sterilized active ingredients into a sterile vehicle which contains the basic dispersion medium and the required other ingredients from those enumerated above. In the case of sterile powders for the preparation of sterile injectable solutions, the preferred methods of preparation are vacuum-drying and freeze-drying techniques, which yield a powder of the active ingredient, plus any additional desired ingredient from a previously sterile-filtered solution thereof.

As used herein, the term “pharmaceutically acceptable” refers to those compounds, materials, compositions, and/or dosage forms which are, within the scope of sound medical judgment, suitable for contact with the tissues of human beings and animals without excessive toxicity, irritation, allergic response, or other problem complications commensurate with a reasonable benefit/risk ratio. The term “pharmaceutically acceptable carrier,” means a pharmaceutically acceptable material, composition or vehicle, such as a liquid or solid filler, diluent, excipient, solvent or encapsulating material, involved in carrying or transporting a chemical agent.

As used herein, “pharmaceutically acceptable salts” refers to derivatives of the disclosed compounds wherein the parent compound is modified by converting an existing acid or base moiety to its salt form. Examples of pharmaceutically acceptable salts include, but are not limited to, mineral or organic acid salts of basic residues such as amines; alkali or organic salts of acidic residues such as carboxylic acids; and the like. Pharmaceutically acceptable salts include the conventional non-toxic salts or the quaternary ammonium salts of the parent compound formed, for example, from non-toxic inorganic or organic acids. The pharmaceutically acceptable salts can be synthesized from the parent compound which contains a basic or acidic moiety by conventional chemical methods.

Some variation in dosage will necessarily occur depending on the condition of the subject. The person responsible for administration will, in any event, determine the appropriate dose for the individual subject. An effective amount of therapeutic or prophylactic composition is determined based on the intended goal. The term “unit dose” or “dosage” refers to physically discrete units suitable for use in a subject, each unit containing a predetermined quantity of the composition calculated to produce the desired responses discussed above in association with its administration, i.e., the appropriate route and regimen. The quantity to be administered, both according to number of treatments and unit dose, depends on the effects desired. Precise amounts of the composition also depend on the judgment of the practitioner and are peculiar to each individual. Factors affecting dose include physical and clinical state of the subject, route of administration, intended goal of treatment (alleviation of symptoms versus cure), and potency, stability, and toxicity of the particular composition.

Upon formulation, solutions will be administered in a manner compatible with the dosage formulation and in such amount as is therapeutically or prophylactically effective. The formulations are easily administered in a variety of dosage forms, such as the type of injectable solutions described above.

Typically, for a human adult (weighing approximately 70 kilograms), from about 0.1mg to about 3000 mg (including all values and ranges there between), or from about 5 mg to about 1000 mg (including all values and ranges there between), or from about 10 mg to about 100 mg (including all values and ranges there between), of a compound are administered. It is understood that these dosage ranges are by way of example only, and that administration can be adjusted depending on the factors known to the skilled artisan.

In certain embodiments, a subject is administered about, at least about, or at most about 0.01, 0.02, 0.03, 0.04, 0.05, 0.06, 0.07, 0.08, 0.09, 0.1, 0.2, 0.3, 0.4, 0.5, 0.6, 0.7, 0.8, 0.9, 1.0, 1.1, 1.2, 1.3, 1.4, 1.5, 1.6, 1.7, 1.8, 1.9, 2.0, 2.1, 2.2, 2.3, 2.4, 2.5, 2.6, 2.7, 2.8, 2.9, 3.0, 3.1, 3.2, 3.3, 3.4, 3.5, 3.6, 3.7. 3.8, 3.9, 4.0, 4.1, 4.2, 4.3, 4.4, 4.5, 4.6, 4.7, 4.8, 4.9, 5.0, 5.1, 5.2, 5.3, 5.4, 5.5, 5.6, 5.7, 5.8, 5.9, 6.0, 6.1, 6.2, 6.3, 6.4, 6.5, 6.6, 6.7, 6.8, 6.9, 7.0, 7.1, 7.2, 7.3, 7.4, 7.5, 7.6, 7.7, 7.8, 7.9, 8.0, 8.1, 8.2, 8.3, 8.4, 8.5, 8.6, 8.7, 8.8, 8.9, 9.0, 9.1, 9.2, 9.3, 9.4, 9.5, 9.6, 9.7, 9.8, 9.9, 10.0, 10.5, 11.0, 11.5, 12.0, 12.5, 13.0, 13.5, 14.0, 14.5, 15.0, 15.5, 16.0, 16.5, 17.0, 17.5, 18.0, 18.5, 19.0. 19.5, 20.0, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 105, 110, 115, 120, 125, 130, 135, 140, 145, 150, 155, 160, 165, 170, 175, 180, 185, 190, 195, 200, 205, 210, 215, 220, 225, 230, 235, 240, 245, 250, 255, 260, 265, 270, 275, 280, 285, 290, 295, 300, 305, 310, 315, 320, 325, 330, 335, 340, 345, 350, 355, 360, 365, 370, 375, 380, 385, 390, 395, 400, 410, 420, 425, 430, 440, 441, 450, 460, 470, 475, 480, 490, 500, 510, 520, 525, 530, 540, 550, 560, 570, 575, 580, 590, 600, 610, 620, 625, 630, 640, 650, 660, 670, 675, 680, 690, 700, 710, 720, 725, 730, 740, 750, 760, 770, 775, 780, 790, 800, 810, 820, 825, 830, 840, 850, 860, 870, 875, 880, 890, 900, 910, 920, 925, 930, 940, 950, 960, 970, 975, 980, 990, 1000, 1100, 1200, 1300, 1400, 1500, 1600, 1700, 1800, 1900, 2000, 2100, 2200, 2300, 2400, 2500, 2600, 2700, 2800, 2900, 3000, 3100, 3200, 3300, 3400, 3500, 3600, 3700, 3800, 3900, 4000, 4100, 4200, 4300, 4400, 4500, 4600, 4700, 4800, 4900, 5000, 6000, 7000, 8000, 9000, 10000 milligrams (mg) or micrograms (mcg) or μg/kg or micrograms/kg/minute or mg/kg/min or micrograms/kg/hour or mg/kg/hour, or any range derivable therein.

A dose may be administered on an as needed basis or every 1, 2, 3, 4, 5, 6, 7, 8, 9, 10, 11, 12, 18, or 24 hours (or any range derivable therein) or 1, 2, 3, 4, 5, 6, 7, 8, 9, or times per day (or any range derivable therein). A dose may be first administered before or after signs of a condition. In some embodiments, the patient is administered a first dose of a regimen 1, 2, 3, 4, 5, 6, 7, 8, 9, 10, 11, 12 hours (or any range derivable therein) or 1, 2, 3, 4, or 5 days after the patient experiences or exhibits signs or symptoms of the condition (or any range derivable therein). The patient may be treated for 1, 2, 3, 4, 5, 6, 7, 8, 9, 10 or more days (or any range derivable therein) or until symptoms of the condition have disappeared or been reduced or after 6, 12, 18, or 24 hours or 1, 2, 3, 4, or 5 days after symptoms of an infection have disappeared or been reduced.

### VII. EXAMPLES

The following examples are included to demonstrate preferred embodiments of the disclosure. It should be appreciated by those of skill in the art that the techniques disclosed in the examples which follow represent techniques discovered by the inventor to function well in the practice of the disclosure, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the disclosure.

### Example 1

### Engineered Collagen-Binding Interleukin-2 Shows Enhanced Antitumor Efficacy and Reduced Adverse Events

Cytokine immunotherapy with interleukin-2 (IL-2) exhibits considerable antitumor activity in animal models and the clinic. Here, the inventors engineered a fusion protein of IL-2 to a collagen-binding domain (CBD) from the von Willebrand Factor (vWF) A3 domain; the fusion protein can be administered intravenously and targets the tumor microenvironment via its leaky vasculature, which allows access of proteins in the blood to the tumor stroma. Here, the inventors show tumor tissue localization of CBD protein. CBD-IL-2 showed decreased serum inflammatory cytokine concentrations in blood serum after injection. CBD-IL-2 significantly delayed tumor growth compared to wild-type IL-2 in murine melanoma and colon cancer models. This simple and translatable approach of an engineered collagen-binding cytokine presents a novel approach to cancer immunotherapeutics.

### A. Results

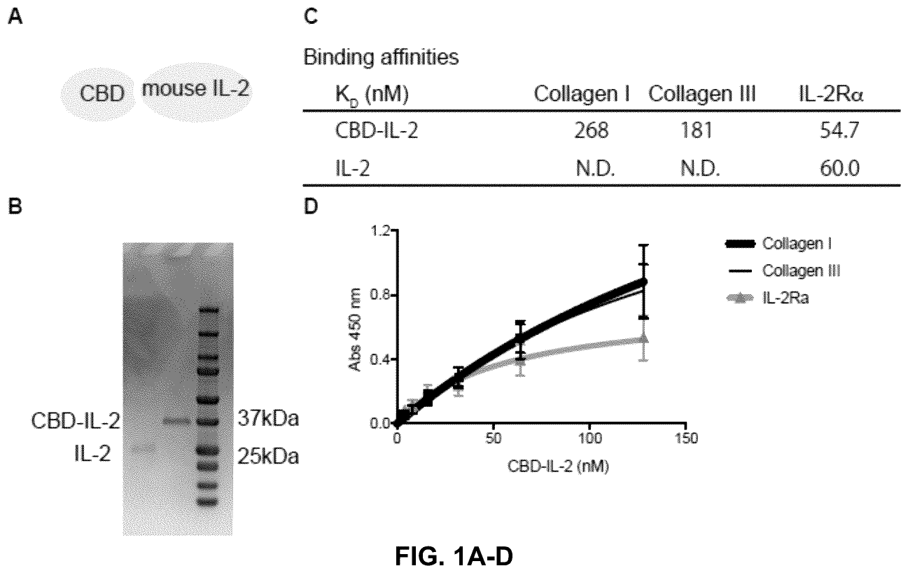

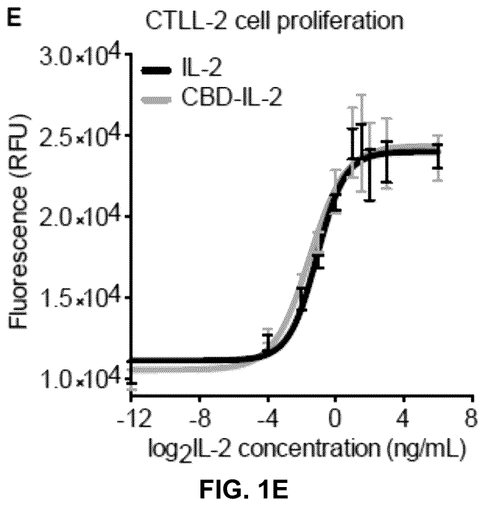

CBD-fused IL-2 bind to collagen and its receptor. The inventors first examined the capacities of CBD-fused IL-2 recombinant protein (CBD-IL-2), to bind collagen in vitro. CBD-fused IL-2 was designed and expressed recombinantly (FIG. 1A). SDS-PAGE revealed that the molecular weight of IL-2 was increased by CBD fusion (FIG. 1B). CBD-IL-2 bound to types I and III collagens with strong binding affinities (nM range dissociation constant (K_{D}) values) (FIG. 1C-1D). In comparison, wild-type IL-2 did not bind to these collagens. Importantly, CBD-IL-2 bound to IL-2Rα with similar K_{D} values as wild-type IL-2. Also, CBD-IL-2 induced cell proliferation of CTLL-2 cell line, which is a IL-2 dependent NK cell line, with similar effect as its wild-type form (FIG. 1E). Taken together, these data showed that CBD-IL-2 binds to collagens without impairment of the target binding capacities of either the CBD or the IL-2 domains.

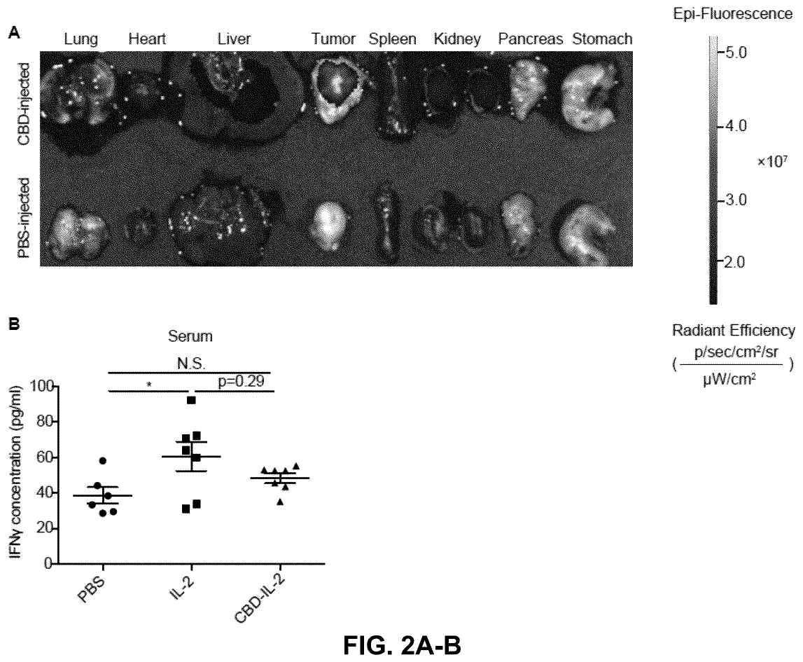

CBD protein localized in the tumor. The inventors performed an in vivo bio-distribution analysis to determine if CBD localizes in the tumor microenvironment after intra-venous (i.v.) injection through binding to endogenous collagen. MMTV-PyMT breast cancer was inoculated in the FVB mouse. When the tumor volume reached 500 mm³, DyLight 800 labeled CBD protein was injected. Two days after injection, the tumor and organs including heart, lung, kidney, liver, spleen, and stomach were harvested. Fluorescent detection revealed that CBD protein preferentially localized in the tumor (FIG. 2A).

CBD fusion decreases treatment-related adverse events. Since CBD protein localized within the tumor, it was hypothesized that the side-effects of CBD-IL-2 after injection would be lower compared to IL-2, due to competition by the tumor and corresponding lowering of systemic exposure. CBD-IL-2 and IL-2 were administrated 4 days after B16F10 melanoma inoculation, then cytokine concentrations in the serum were examined 24 hr following injection. Wild-type IL-2 administration increased IFNy concentrations in serum, whereas CBD-IL-2 did not (FIG. 2B). This result indicates that the CBD fusion may decrease the systemic toxicity of IL-2.

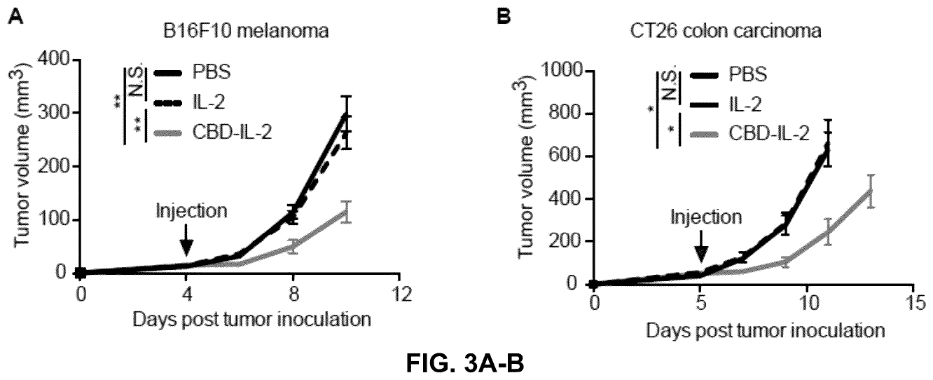

CBD-IL-2 significantly suppresses growth of B16F10 and CT-26 tumors compared to IL-2. The inventors examined the antitumor efficacy of CBD-IL-2 using B16F10 melanoma and CT26 colon carcinoma. Four days after B16F10 cell inoculation, 6 μg IL-2 or 12 μg (equivalent molar) CBD-IL-2 were injected. At this dose, wild-type IL-2 treatment did not show a clear antitumor effect, while CBD-IL-2 treatment induced smaller tumor sizes (FIG. 3A). Five days after CT26 cell inoculation, 6 μg IL-2 or 12 μg (equivalent molar) CBD-IL-2 were injected. CBD-IL-2 again slowed the tumor growth of CT26, whereas IL-2 did not (FIG. 3B). These set of data indicated that CBD-IL-2 therapy has superior antitumor effects compared to its wild-type form.

The strategy of localized cancer therapy can be classified as active targeting and passive targeting (Danhier et al., J Control Release 148:135-46, 2010). Antibody-drug conjugates are an example of active targeting. Targeting is based on the attachment of drugs to specific ligands (e.g., antibodies) that are tumor or tumor cell-specific (Chari et al., Angew Chem Int Ed Engl 53:3796-827, 2014), facilitating delivery of cancer drugs specifically to tumor cell surfaces. An example of passive targeting is a drug embedded in a nanoparticle carrier. Nanoparticles are expected to have a prolonged half-life in the blood, leading to accumulation in tumor where the vasculature is leaky via the enhanced permeability and retention (EPR) effect (Maeda et al., J Control Release 65:271-84, 2000; Swartz and Fleury, Annu Rev Biomed Eng 9:229-56, 2007). Therefore, passive targeting is based on the longevity of the pharmaceutical carrier in the blood and its accumulation in pathological sites with irregular vasculature and thus enhanced accumulation.

The collagen binding domain (CBD)-based drug targeting approach proposed in this application is similar to active targeting, but also exploits the leaky structure of tumor vessels (Nagy et al., British journal of cancer 100:865, 2009; in the normal tissue, very little collagen is exposed (Dubois et al., Blood 107:3902-06, 2006; Bergmeier and Hynes, Cold Spring Harb Perspect Biol 4:a005132, 2012), but in the tumor, with its leaky vasculature, collagen is indeed exposed (Liang et al., Journal of controlled release 209:101-09, 2015; Liang et al., Sci Rep 6:18205, 2016; Yasunaga et al., Bioconjugate chemistry 22:1776-83, 2011; Xu et al., The Journal of cell biology 154:1069-80, 2001; Swartz and Lund, Nat Rev Cancer 12:210-19, 2012). Thus, the CBD-fusions are tumor microenvironment-specific, yet not via targeting a molecule that is specifically located in the tumor (indeed, collagen is nearly everywhere), but rather that is only exposed via the tumor's leaky vasculature. Also, this can be a hybrid approach utilizing both advantages.

The vWF A3 domain and collagen association is an initiator of the thrombosis cascade, thus this binding commonly occurs in human body (Shahidi, Advances in experimental medicine and biology 906:285-306, 2017). In this study, the inventors have developed cancer immunotherapy, targeting cancer microenvironment using the vWF A3 domain as CBD.

Previously, a tumor matrix targeting approach that fuses a single chain antibody fragment against a tumor-specific fibronectin domain and cytokines has been tested in animal models and clinical trials (Carnemolla et al., Blood 99:1659-65, 2002; Eigentler et al., Clinical cancer research 17:7732-42, 2011; Ferrari et al., Drug Discov Today 21:172-79, 2016). The fibronectin extra-domain A (EDA) and EDB domains are expressed in tumor but not in the normal tissue (Rybak et al., Cancer Res 67:10948-957, 2007; Villa et al., Int J Cancer 122:2405-13, 2008). Single chain antibodies against the fibronectin EDA and EDB domains localize within tumor after systemic injection through this tumor-specific location of the EDA and EDB targets (Carnemolla et al., Blood 99:1659-65, 2002; Rybak et al., Cancer Res 67:10948-957, 2007). A fusion protein of the single chain antibody and IL-2 showed enhanced antitumor efficacy compared to normal IL-2 in a mouse model (Carnemolla et al., Blood 99:1659-65, 2002).

Collagen is the most abundant protein in the human body (Addi et al., Tissue Engineering Part B: Reviews, 2016; Di Lullo et al., Journal of Biological Chemistry 277:4223-31, 2002, especially tumors contain more collagen than normal tissue (Zhou et al., J Cancer 8:1466-76, 2017; Provenzano et al., BMC Med 6:11, 2008). Thus, in the CBD approach, the target protein should be more abundant compared to the EDA and EDB domains of fibronectin. Liang et al. have reported that fusing a collagen-binding short peptide (TKKLRT (SEQ ID NO:14)) with an anti-epithelial growth factor receptor (EGFR) Fab or single-chain antibody improves tumor tissue localization of the antibody compared to naive anti-EGFR Fab or single-chain antibody when injected i.p. (Liang et al., Journal of controlled release 209:101-09, 2015; Liang et al., Sci Rep 6:18205, 2016). This TKKLRT peptide was discovered by phage display, with reported K_{D} values or the half-maximal effective concentration (EC50) toward collagen type I being 0.5-6 μM (Addi et al., Tissue Engineering Part B: Reviews, 2016). Also, TKKLRT-anti-EGFR Fab exhibited enhanced anti-tumor efficacy compared to naive anti-EGFR antibody. With EGFR, this mechanism is mediated by direct inhibition of proliferation of cancer cells, but not immuno-modulation (Martinelli et al., Clin Exp Immunol 158:1-9, 2009). The inventors' approach uses CBD proteins that are naturally existing in the body and are thus not immunogenic. CBD protein such as the vWF A3 protein binds to multiple collagen types unlike more specific ligand such as peptides, antibodies or antibody fragments. Also, the vWF A3 domain protein has high affinity (nM range Kp) towards multiple types of collagens (Addi et al., Tissue Engineering Part B: Reviews, 2016). Thus, the approach is novel both in the methodology (high affinity protein domains derived from protein that naturally exist in the body, versus low affinity peptide domains) and in the biological approach (targeting the protein that is abundant in the body but that is only exposed in the tumors via its leaky vasculature).

The inventors have shown that the CBD-IL-2 fusion did not increase IFNγ concentrations in blood serum whereas the wild-type IL-2 did. Considering CBD-mediated tumor targeting, this could be because of the sequestration of IL-2 within the tumor by competition. CBD-IL-2 localizes within the tumor and thus reduces the concentration in the blood circulation, thus potentially maintaining systemic immune homeostasis by avoiding systemic immune cell activation. CBD-fusion may even allow decreases in the administered dose, because the inventors observed tumor growth delay at low dosages where the unmodified IL-2 had no effect. These data are encouraging with regard to treatment of patients who have discontinued cancer immunotherapy due to adverse events.

CBD-fused IL-2 demonstrated higher antitumor effects compared to the wild-type form in two tumor models. These data suggest that the collagen targeting approach by the vWF A3 domain is generally applicable to multiple tumors. IL-2 is known to have an antitumor effect through amplifying CD8⁺ T cells and NK cells. Thus, with tumor targeting, CBD-IL-2 might more efficiently increase the numbers of tumor-infiltrating T cells and NK cells compared to its unmodified form.

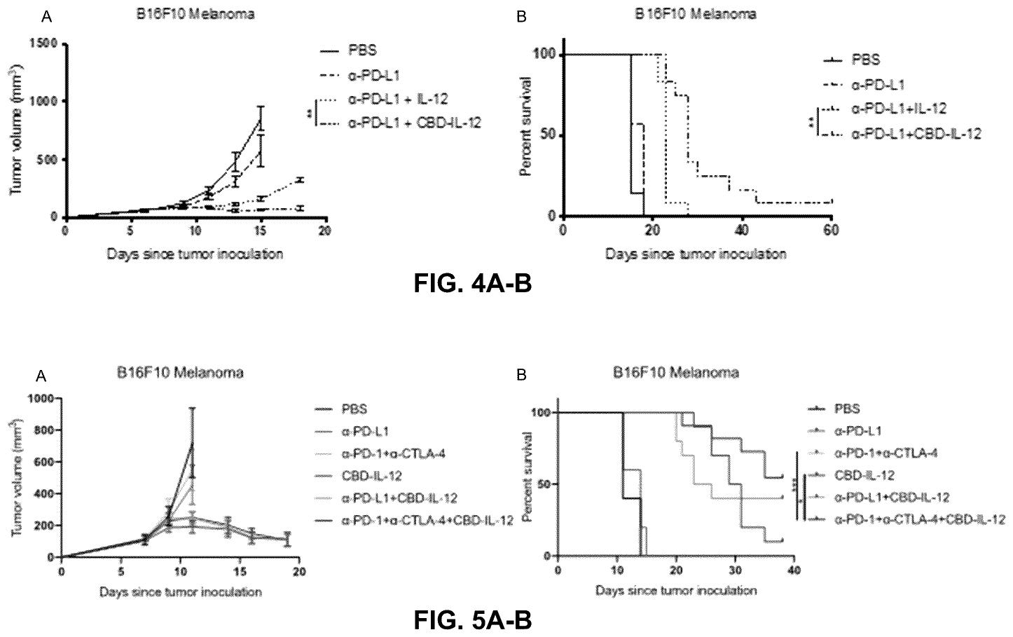

Since checkpoint inhibitors are approved for melanoma patients, and B16F10 melanoma is checkpoint-unresponsive model, the inventors were interested in whether CBD-IL-12 can increase the antitumor efficacy of α-PD-L1 antibody. Mice were treated in three cycles, every 10 days starting on day 7. IL-12 and α-PD-L1 injections were performed on consecutive days with IL-12 being administered first. α-PD-L1 alone had little effect on tumor growth (FIG. 4A). Although both IL-12 and CBD-IL-12 significantly reduced tumor growth in combination with α-PD-L1 compared to either PBS-treated or antibody-treated groups, CBD-IL-12 did so more effectively. CBD-IL-12-treated mice had significantly prolonged overall survival (FIG. 4B), with 1 out of 12 mice completely curing the disease.

To investigate whether CBD-IL-12 synergizes with checkpoint-blocking antibodies, CBD-IL-12 monotherapy was compared to CBD-IL-12 in combination with α-PD-L1 alone or α-PD-1+α-CTLA-4. Mice failed to respond to either α-PD-L1 or α-PD-1+α-CTLA-4, whereas robust regressions in tumor sizes were observed for CBD-IL-12 alone and for CBD-IL-12 in combination with checkpoint-blocking antibodies in the early time points (FIG. 5A). However, overall survival was significantly improved when mice received CBD-IL-12 in combination with α-PD-1+α-CTLA-4, yielding 6 out of 11 complete responders (FIG. 5B). 4 out of 10 mice completely cured the disease when CBD-IL-12 was combined with α-PD-L1, whereas only 1 out of 10 mice was cured in CBD-IL-12 monotherapy group.

In conclusion, the inventors found that the antitumor effect of IL-2 was enhanced when a collagen-binding property was installed. Fusion of the vWF A3 CBD enhanced IL-2. CBD-IL-2 significantly delayed tumor growth in multiple tumor models. This simple approach of an engineered collagen-binding cytokine immunotherapy may hold potential for clinical translation as a targeted cancer therapeutic.

### B. Materials and methods

Production and purification of recombinant vWF A3 domain and mouse IL-2 protein. Protein production and purification were performed as described previously (Martino et al., Science 343:885-88, 2014). The sequence encoding for the human vWF A3 domain residues Cys1670-Gly1874 (907-1111 of mature vWF), mouse IL-2, the human vWF A3 domain and mouse IL-2 fusion protein was synthesized and subcloned into the mammalian expression vector pcDNA3.1(+) by Genscript. A sequence encoding for 6 His was added at the N-terminus for further purification of the recombinant protein. Suspension-adapted HEK-293F cells were routinely maintained in serum-free FreeStyle 293 Expression Medium (Gibco). On the day of transfection, cells were inoculated into fresh medium at a density of 1×10⁶ cells/ml. Two μg/ml plasmid DNA, 2 μg/ml linear 25 kDa polyethylenimine (Polysciences), and OptiPRO SFM media (4% final concentration, Thermo Fisher) were sequentially added. The culture flask was agitated by orbital shaking at 135 rpm at 37° C. in the presence of 5% CO₂. Six days after transfection, the cell culture medium was collected by centrifugation and filtered through a 0.22 μm filter. Culture media was loaded into a HisTrap HP 5 ml column (GE Healthcare), using an ÄKTA pure 25 (GE Healthcare). After washing of the column with wash buffer (20 mM imidazole, 20 mM NaH₂PO₄, 0.5 M NaCl, pH 7.4), protein was eluted with a gradient of 500 mM imidazole (in 20 mM NaH₂PO₄, 0.5 M NaCl, pH 7.4). The elution solution was further purified with size exclusion chromatography using a HiLoad Superdex 200PG column (GE healthcare). All purification steps were carried out at 4° C. The expression of laminin LG domain was determined by western blotting using anti-His tag antibody (BioLegend) and the proteins were verified as >90% pure by SDS-PAGE.

vWF A3 domain protein-
(SEQ ID NO: 34)
CSQPLDVILLLDGSSSFPASYFDEMKSFAKAFISKANIGPRLTQVSVLQY
GSITTIDVPWNVVPEKAHLLSLVDVMQREGGPSQIGDALGFAVRYLTSEM
HGARPGASKAVVILVTDVSVDSVDAAADAARSNRVTVFPIGIGDRYDAAQ
LRILAGPAGDSNVVKLQRIEDLPTMVTLGNSFLHKLCSGFVRICTGHHHH
HH
Mouse IL-2-
(SEQ ID NO: 35)
PTSSSTSSSTAEAQQQQQQQQQQQQHLEQLLMDLQELLSRMENYRNLKLP
RMLTFKFYLPKQATELKDLQCLEDELGPLRHVLDLTQSKSFQLEDAENFI
SNIRVTVVKLKGSDNTFECQFDDESATVVDFLRRWIAFCQSIISTSPQHH
HHHH
The vWF A3 domain and mouse IL-2 fusion protein-
(SEQ ID NO: 36)
CSQPLDVILLLDGSSSFPASYFDEMKSFAKAFISKANIGPRLTQVSVLQY
GSITTIDVPWNVVPEKAHLLSLVDVMQREGGPSQIGDALGFAVRYLTSEM
HGARPGASKAVVILVTDVSVDSVDAAADAARSNRVTVFPIGIGDRYDAAQ
LRILAGPAGDSNVVKLQRIEDLPTMVTLGNSFLHKLCSGFVRIGGGSGGG
SPTSSSTSSSTAEAQQQQQQQQQQQQHLEQLLMDLQELLSRMENYRNLKL
PRMLTFKFYLPKQATELKDLQCLEDELGPLRHVLDLTQSKSFQLEDAENF
ISNIRVTVVKLKGSDNTFECQFDDESATVVDFLRRWIAFCQSIISTSPQH
HHHHH

Sodium dodecyl sulfate acrylamide gel electrophoresis (SDS-PAGE). The measurement is performed as described previously (Ishihara et al., Sci Transl Med 9:doi:10.1126/scitranslmed.aan0401, 2017). SDS-PAGE was performed on 4-20% gradient gels (Bio-Rad). After electrophoresis, gels were stained with SimplyBlue SafeStain (Thermo Fisher Scientific) according to manufacturer's instruction. Gel images were acquired with the ChemiDoc XRS+ system (Bio-Rad).

Detection of CBD-IL-2 binding to collagen proteins. The measurement is performed as described previously (Ishihara et al., Sci Transl Med 9:doi:10.1126/scitranslmed.aan0401, 2017). 96-well ELISA plates (Greiner Bio One) were coated with 10 μg/mL collagen I (EMD Millipore), collagen III (EMD Millipore), or 1 μg/mL recombinant mouse IL-2Rα (SinoBiological) in PBS for 1 h at 37° C., followed by blocking with 2% BSA in PBS with 0.05% Tween 20 (PBS-T) for 1 hr at RT. Then, wells were washed with PBS-T and further incubated with 10 μg/mL CBD- or unmodified-IL-2 for 1 hr at RT. After 3 washes with PBS-T, wells were incubated for 1 hr at RT with HRP-conjugated antibody against rat IgG (Jackson ImmunoResearch). After washes, bound CBD-IL2 and IL-2 were detected with tetramethylbenzidine substrate by measurement of the absorbance at 450 nm with subtraction of 570 nm. The apparent dissociation constant (KD) values were obtained by nonlinear regression analysis in Prism software (v7, GraphPad Software) assuming one-site specific binding.

Proliferation assay. CTLL-2 cells (ATCC) were cultured in RPMI 1640 (ATCC), supplemented with heat inactivated fetal bovine serum, L-glutamine, sodium pyruvate, and penicillin-streptomycin, and recombinant mouse IL-2 (Peprotech). Cells were passaged twice a week to a density of 10,000 cells/mL. For proliferation assays, cells were seeded at 100,000 cells/mL, and mouse IL-2 and CBD-IL-2 were added at indicated concentrations on an IL-2 basis, in a final volume of 100 μL. Cells were grown for 48 hrs. Proliferation was conducted by using CyQUANT Cell Proliferation Assay Kit (Invitrogen) according to manufacturer's instructions. Fluorescence was measured using a BioTek Cytation 3 Cell Imaging Multi-Mode Reader (fisher scientific). A dose response curve was fit via nonlinear regression using GraphPad Prism 7software (GraphPad).

Mice and cell lines. The mice and cell lines were prepared as described previously (Ishihara et al., Sci Transl Med 9:doi:10.1126/scitranslmed.aan0401, 2017). C57BL/6, FVB, and Balb/c mice, age 8 to 12 weeks, were obtained from the Jackson laboratories. Experiments were performed with approval from the Institutional Animal Care and Use Committee of the University of Chicago. B16F10 cells and CT26 cells were obtained from the American Type Culture Collection and cultured according to the instructions. All cell lines were checked for mycoplasma contamination by a pathogen test IMPACT I (IDEXX BioResearch).

In vivo bio-distribution study. The vWF A3 domain protein was fluorescently labeled using DyLight 800 NHS ester (Thermo Fisher) and unreacted dye was removed by a Zebaspin spin column (Thermo Fisher) according to the manufacture's instruction. A total of 8×10⁵ MMTV-PyMT cells re-suspended in 50 μL of PBS were injected subcutaneously into the mammary fat pad on the right side of each FVB mouse. When tumor reached 500 mm³, 50 μg of DyLight 800 labeled CBD was injected i.v. Mice organs were extracted and imaged 48 hours after injection with the Xenogen IVIS Imaging System 100 (Xenogen) under the following conditions: f/stop: 2; optical filter excitation 740 nm; excitation 800 nm; exposure time: 5 sec; small binning.

Serum cytokine concentration analysis. The measurement is performed as described previously (Ishihara et al., Sci Transl Med 9:doi:10.1126/scitranslmed.aan0401, 2017). 5×10⁵ B16F10 melanoma cells were injected intradermally on left side of the back of each 12 week old C57BL/6 mouse (The Jackson Laboratory). After 4 days, mice received 6 μg of IL-2 and 12 μg of CBD-IL-2. On day 5, blood samples were collected in tubes, followed by overnight incubation at 4° C. Cytokine concentrations in serum were measured by Ready-SET-Go! ELISA kits (eBioscience) according to the manufacture's protocol.

Anti-tumor efficacy of CBD-IL-2 on B16F10 tumor. The measurement is performed as described previously (Ishihara et al., Sci Transl Med 9:doi:10.1126/scitranslmed.aan0401, 2017). A total of 5×10⁵ B16F10 cells re-suspended in 50 μL of PBS were inoculated intradermally on the left side of the back of each C57BL/6 mouse. After 4 days, mice were injected with IL-2 (6 μg) or CBD-IL-2 (12 μg) i.v. Tumors were measured with a digital caliper starting 4 days after tumor inoculation, and volumes were calculated as ellipsoids, where V=4/3×3.14×depth/2×width/2×height/2. Mice were sacrificed at the point when either tumor volume had reached over 500 mm³.

Antitumor efficacy of CBD-IL-2 on CT26 tumor. A total of 5×10⁵ CT26 cells re-suspended in 50 μL of PBS were inoculated intradermally on the left side of the back of each Balb/c mouse. After 5 days, mice were injected i.v. with IL-2 (6 μg) or CBD-IL-2 (12 μg). Tumors were measured with a digital caliper starting 5 days after tumor inoculation as described above. Mice were sacrificed at the point when either tumor volume had reached over 500 mm³.

Statistical analysis. All experiments are replicated at least twice. For the animal study, mice were randomized into treatment groups within a cage immediately before IL-2 and CBD-IL-2 injection and treated in the same way. The survival endpoint was reached when the tumor size became over 500 mm³ for B16F10 and CT26 tumors. The n values used to calculate statistics are indicated in figure legends. Statistically significant differences between experimental groups were determined using Prism software (v7, GraphPad). Where one-way ANOVA followed by Tukey's HSD post hoc test was used, variance between groups was found to be similar by Brown-Forsythe test. For single comparisons, a two-tailed Student's/-test was used. Survival curves were analyzed by using the log-rank (Mantel-Cox) test. The n values used to calculate statistics are indicated in figure legends. The symbols * and ** indicate P values less than 0.05 and 0.01, respectively; N.S., not significant.

Additional cytokines to be tested. Other than IL-2, a number of cytokines including chemokines have been shown to have anti-tumor efficacy and are currently being tested in the clinical trials (Tokunaga et al., Cancer treatment reviews 63:40-47, 2017; Lin et al., Cancers (Basel) 6:1098-110, 2014; Akdis et al., The Journal of allergy and clinical immunology 127:701-21, 2011; Waldmann, Nature reviews. Immunology 6:595-601, 2006). The inventors expect that addition of CBD sequence in the N- or C-terminus of the cytokine enables efficient tumor targeting, as the inventors have shown with CBD-IL-2 in the previous part. The inventors will test cytokines that multiply T cells: IFNα, IFNβ, IL-15, IL-15 super agonist (fusion protein of IL-15 and IL-15Rα), and IL-21, or NK cells: IL-12 (Akdis et al., The Journal of allergy and clinical immunology 127:701-21, 2011; Waldmann, Nature reviews. Immunology 6:595-601, 2006). The inventors will also test VEGF-C, which is a cytokine that induces lymphangiogenesis, leading to increased immune cells infiltration (Fankhauser et al., Sci Transl Med 9:doi:10.1126/scitranslmed.aal4712, 2017; Lund et al., Cell Rep 1:191-99, 2012). Similarly, the inventors will test chemokines (e.g., XCL1, CCL4, CCL21, CXCL9, and CXCL10) that reportedly recruit anti-tumor immune cells into tumor microenvironment (Tokunaga et al., Cancer treatment reviews 63:40-47, 2017; Lin et al., Cancers (Basel) 6:1098-110, 2014).

Additional CBD to be tested. Other than the vWF A3 domain, protein (the vWF A1domain (SEQ ID NO:11) and decorin (SEQ ID NO:15)), and a peptide (TKKLRT (SEQ ID NO:14)) have been shown to bind to collagen (Addi et al., Tissue Engineering Part B: Reviews, 2016). Because the vWF A1 domain and decorin are similar molecular weight proteins as the vWF A3 domain protein and show high binding affinity similar to the vWF A3 domain, it is expect that addition of these the vWF A1 domain and decorin sequence in the N- or C-terminus of the cytokines also target tumor microenvironment, similarly to that shown with CBD-IL-2. However, it is anticipated that addition of TKKLRT sequence in the N- or C-terminus of the cytokine would not show tumor microenvironment targeting capacity due to its small molecular weight and low collagen affinity (μM range of K_{D}) (Addi et al., Tissue Engineering Part B: Reviews, 2016). Thus, addition of TKKLRT sequence would not alter the antitumor activity of the cytokine.

Production and purification of CBD-fused cytokine. As described in the CBD-IL-2 part above, the sequence encoding for CBD will be cloned and subcloned into the mammalian expression vector pcDNA3.1(+). Histidine-tag will be added on the N- or C-terminus of the protein sequence. The CBD-fused cytokine recombinant protein will be expressed in suspension-adapted HEK-293F cells in serum-free FreeStyle 293 Expression Medium. The produced recombinant protein will be purified using a histidine-tagged protein purification column and size-exclusion.

Tumor inoculation and immunomodulatory antibody treatment through intra-venous injection. Tumor cells (e.g., B16F10) will be inoculated intradermally (id). After tumors become visible, mice will receive intravenous injections of CBD- or wild-type-cytokines through the tail vein. Tumor size will be monitored until it reaches the euthanasian criteria. Here, the inventors expect that iv injection of CBD-cytokines will show enhanced tumor tissue localization and retention through binding to collagen in the tumor microenvironment. Through a similar mechanism to what is described above in IL-2 part, CBD-fusion will increase cytokine concentration within the tumor microenvironment, leading to reduced incidents of side-effects and enhance anti-tumor efficacy of cytokine/chemokine.

Example 2

### Recruitment of CD103⁺ DCS via Tumor-Targeted Chemokine Delivery Enhances Efficacy of Checkpoint Inhibitor Immunotherapy

### A. Results

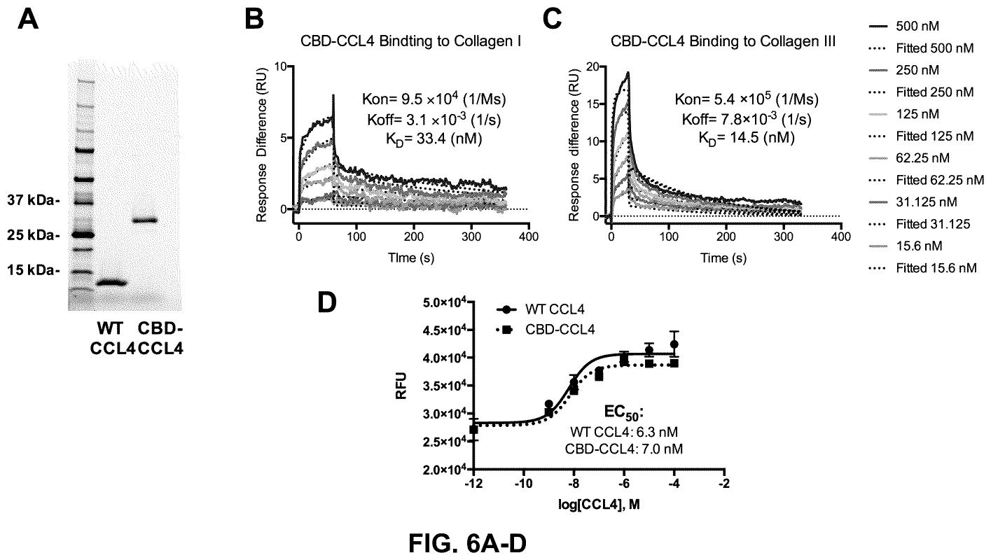

CBD-CCL4 recombinant protein was produced using mammalian protein expression techniques similar to previous reports (Ishihara et al., Sci Transl Med, 2019). Following production and purification using affinity and size-exclusion chromatography, CBD-CCL4 was evaluated using SDS-polyacrylamide gel electrophoresis (SDS-PAGE). Fusion with CBD increased the molecular size of CCL4 by approximately 20 kDa compared to native (WT) CCL4, consistent with the size of the A3 domain of vWF (FIG. 6A). Using surface plasmon resonance (SPR), the affinity for collagen I and collagen III was calculated as 33.4 nM and 14.5 nM, respectively (FIG. 6B, 6C). These results agree with previous collagen affinity for CBD-modified immunotherapies (Ishihara et al., Sci Transl Med, 2019). Next, the activity of WT CCL4 and CBD-CCL4 was evaluated using a calcium flux assay, as CCL4 signals through the G protein-coupled receptor (GPCR) CCR5 (Alkhatib, Curr Opin HIV AIDS 4, 96-103, 2009), leading to calcium release upon activation (FIG. 6D). Both native CCL4 and CBD-CCL4 exhibited similar GPCR activation levels, highlighting that CBD-fusion did not alter its ability to signal through CCR5.

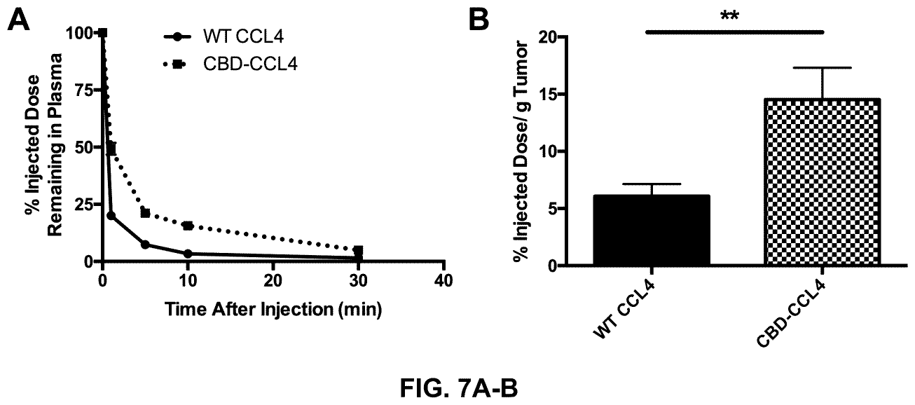

Next, the blood plasma pharmacokinetics of WT CCL4 and CBD-CCL4 were evaluated following intravenous administration in B16F10 tumor-bearing mice. Interestingly, CBD-CCL4 exhibited delayed clearance compared to WT CCL4 (FIG. 7A). The increase in molecular size of the molecule may slow its clearance from blood; this could have important implications for tumor-targeting, as enhanced blood circulation time is often attractive for enhancing delivery of macromolecules to tumors (Pisal et al., J Pharm Sci 99, 2557-75, 2010). Furthermore, enhanced circulation time may allow for additional opportunities to bind to exposed collagen in the leaky tumor microvasculature (Ruoslahti et al., J Cell Biol 188, 759-68, 2010). To confirm that CBD-fusion enhanced tumor delivery of CCL4, biodistribution studies were performed in EMT6 breast cancer-bearing mice following intravenous administration. Importantly, CBD-CCL4 fusion exhibited a 2.4-fold increase in tumor accumulation 30 min following administration (FIG. 7B). These data demonstrate the effective delivery of CBD-CCL4 to the tumor microenvironment.

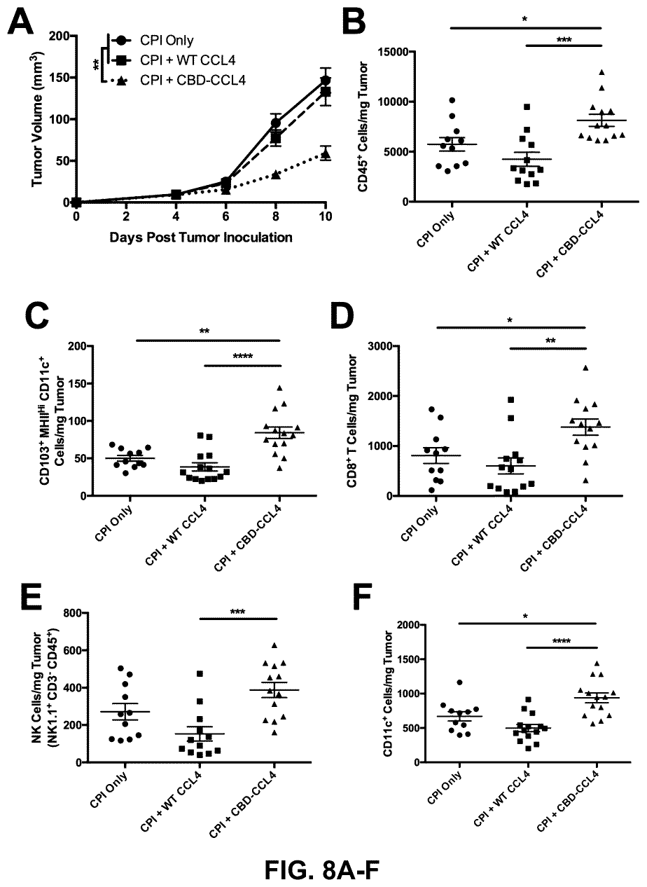

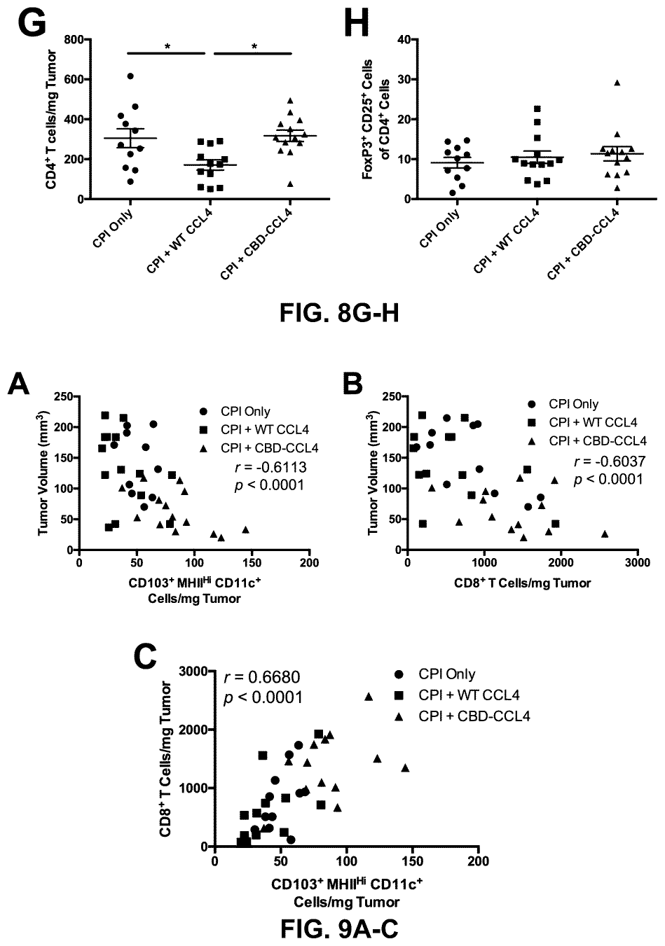

Next, the inventors investigated whether it could enhance tumor immune infiltration, a key factor driving successful responses to CPI therapy. For all subsequent experiments CCL4chemokine therapy was co-administered with CPI therapy comprising of αCTLA4+αPD-L1, a combination treatment strategy for advanced melanoma and non-small cell lung cancer in the clinic (Chae et al., J Immunother Cancer 6, 39, 2018; Gong et al., J Immunother Cancer 6, 8, 2018). CPI therapy alone was included for baseline comparison. Combination CCL4 and CPI therapy were evaluated in B16F10 melanoma, a tumor model shown to be a poor responder to CPI therapy alone (Lechner et al., J Immunother 36, 477-89, 2013). As shown in FIG. 8A, only the combination of CBD-CCL4 (administered via i.v. injection) and CPI therapy (administered via intraperatonieal (i.p.) injection) showed a significant reduction in tumor growth rate. WT CCL4, given in combination with CPI therapy, did not show any reduction in tumor growth rates. These results confirm that targeted chemokine delivery is required to elicit a therapeutic benefit from CCL4.

Because a significant slowing of tumor growth was observed, it was hypothesized that an increase in CD103⁺ DC recruitment to the tumor may be contributing to the anti-tumor immune response. 6 days following administration of the treatment regime, mice were sacrificed, and tumors were harvested and processed for flow cytometry analysis of the immune cell infiltrates in the tumor. Compared to CPI therapy alone and CPI given in combination with WT CCL4, CPI therapy given with CBD-CCL4 significantly increased the number of CD45⁺ immune cells (FIG. 8B) in the tumor, indicating a more inflamed microenvironment. Looking specifically at the immune cell composition, it was observed that the CPI therapy given with CBD-CCL4 led to the highest infiltration of key drivers of anti-tumor immune responses, including CD103⁺ DCs (FIG. 8C), CD8⁺ T cells (FIG. 8D), natural killer (NK) cells (FIG. 8E), as well as total CD11c⁺ DCs (FIG. 8F). CD4⁺ T cells was also significantly improved relative to combination of CPI therapy and WT CCL4 (FIG. 8G). Importantly, no increase in the regulatory T cell (Treg) fraction of CD4⁺ T cells was observed (FIG. 8H), indicating that increases in tumor inflammation did not also significantly alter Treg recruitment.

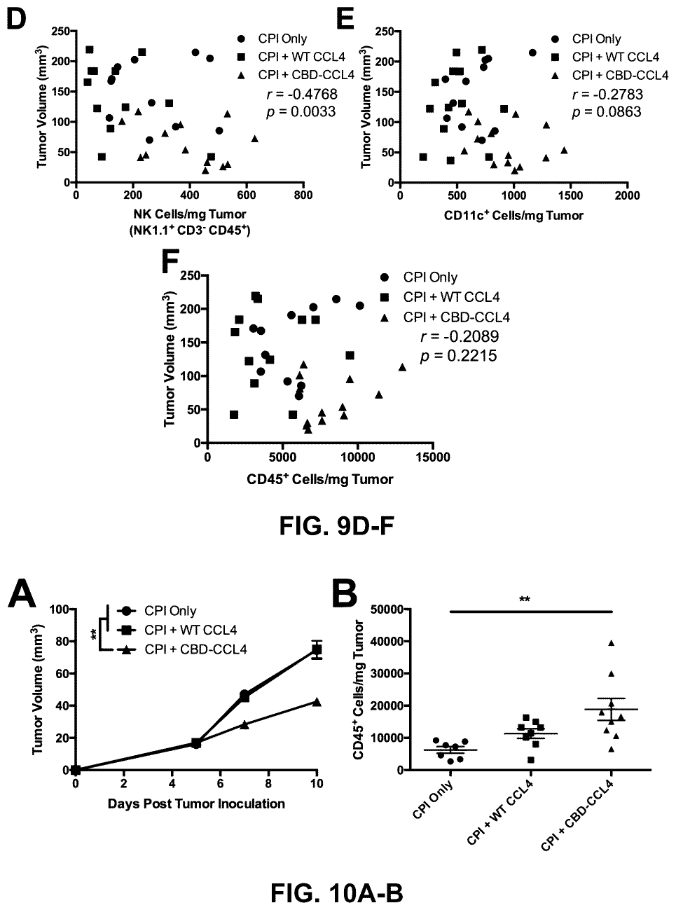

Correlation analysis was performed between the immune cell infiltration and tumor growth to highlight the contribution of each cell population to driving anti-tumor immunity. Correlation between tumor volume and cell infiltration was strongest for CD103⁺ DCs and CD8⁺ T cells (FIG. 9A, 9B), with the greatest cell infiltrate numbers leading to the smallest tumor volumes. As expected, a significant positive correlation between CD103⁺ DCs and CD8⁺ T cells was observed (FIG. 9C), as it has previously been shown that CD103⁺ DCs secrete chemokines necessary for T cell infiltration into the tumor (Spranger et al., Cancer Cell 31, 711-23 e714, 2017). Lesser trends were observed between NK cells and CD11c⁺ DCs (FIG. 9D, 9E), highlighting that these cell types are important for tumor growth control, albeit less so than CD103⁺ DCs and CD8⁺ T cells. Furthermore, a less striking correlation was observed between total CD45⁺ immune cells and tumor growth (FIG. 9F), indicating that total immune infiltration alone is not strong enough to drive anti-tumor immunity. Rather, the specific cell types driving anti-tumor immunity must be recruited to maximize therapeutic effect.

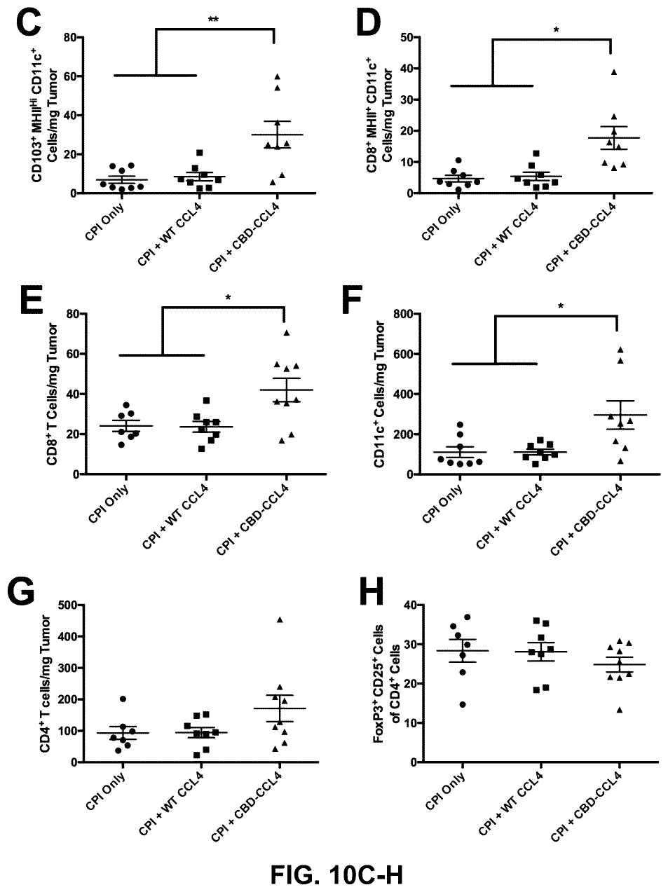

To follow up on the immune infiltrate responses observed in B16F10 melanoma, similar analysis were performed in the EMT6 breast cancer model. The EMT6 model, while moderately responsive to CPI therapy, is categorized as an immune excluded tumor model (Mariathasan et al., Nature 554, 544-48, 2018). Therefore, it was hypothesized that tumor-targeted CCL4 delivery may further enhance CD103⁺ DC recruitment and further improve efficacy of CPI therapy. Similar to the results observed in B16F10 melanoma, only the combination of CPI therapy and CBD-CCL4 exhibited a significant reduction in tumor growth (FIG. 10A). WT CCL4 given in combination with CPI therapy showed no significant improvement relative to CPI therapy alone. Detailed analysis of the immune cell infiltrates using flow cytometry found that CBD-CCL4 in combination with CPI therapy exhibited a significant increase in the total number of CD45⁺ immune cells (FIG. 10B). Specifically, CBD-CCL4 combination therapy mediated the highest recruitment of CD103⁺ DCs, CD8α⁺ cross-presenting DCs, and CD8⁺ T cells (FIG. 10C-10E). Significant increases in total CD11c⁺ DCs was also observed (FIG. 10F). CBD-CCL4 therapy did not increase recruitment of CD4⁺ T cells, nor did it enhance the fraction of Tregs in the CD4⁺ T cell compartment (FIG. 10G, 10H). Taken together, these results once again highlight the importance of targeted CCL4 delivery in the recruitment of key cell populations to enhance the efficacy of CPI therapy.

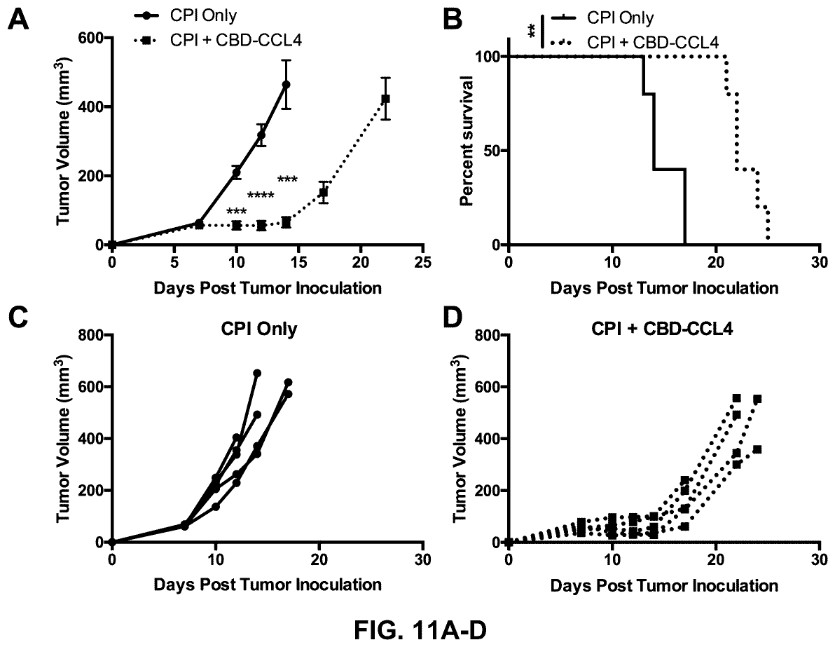

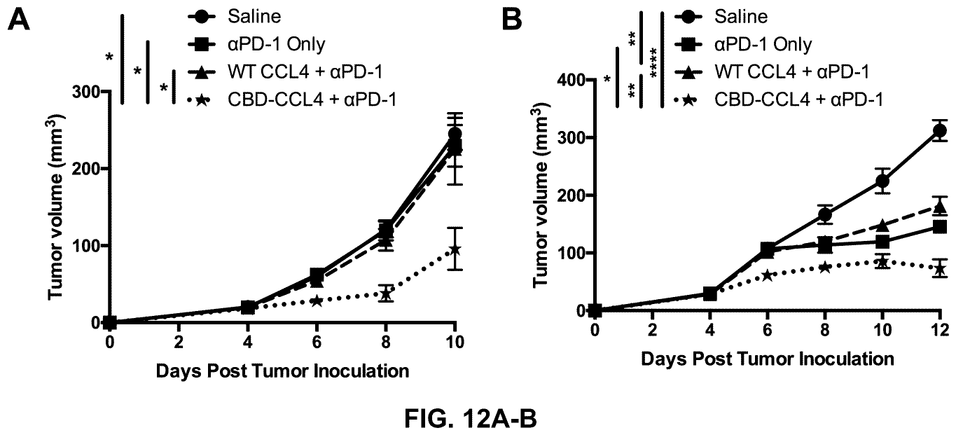

Encouraged by the results seen with combination therapy in B16F10 melanoma when treating tumors at early time points, further investigation as to whether CPI therapy in combination with CBD-CCL4 could slow growth of established B16F10 tumors was performed. Tumors were allowed to progress until their volume was greater than 50 mm³, after which tumors were treated with CPI therapy alone or CPI+CBD-CCL4. Importantly, combination therapy significantly slowed tumor growth following a single administration; furthermore, combination therapy prolonged mouse survival, with mice co-administered CPI therapy with CBD-CCL4 surviving for an average of 22 days, compared to 14 days for CPI therapy alone (FIG. 11). Extending from these results, the inventors investigated as to whether CBD-CCL4 could synergize with anti-programmed cell death protein 1 (αPD-1, CD279), another clinically approved immunotherapy for a number of indications, including melanoma, non-small cell lung cancer, bladder cancer, renal cell carcinoma, and hepatocellular carcinoma, among others (Gong et al., J Immunother Cancer 6, 8, 2018). Using two syngenic colon cancer models, CT26 and MC38, it was found that combination of αPD-1 therapy and CBD-CCL4 mediated the slowest tumor growth rates in both models, significantly enhancing therapeutic benefit relative to antibody therapy alone or combination with WT CCL4 (FIG. 12). These results highlight that CBD-CCL4 can be combined with multiple CPI antibody therapies to improve therapeutic effect. Furthermore, since the targeting approach used for CBD-CCL4 therapy utilizes the tumor extracellular matrix, as opposed to a specific protein on the surface of tumor cells, this therapy can be applied to a number of different tumor models to improve efficacy of CPI therapy.

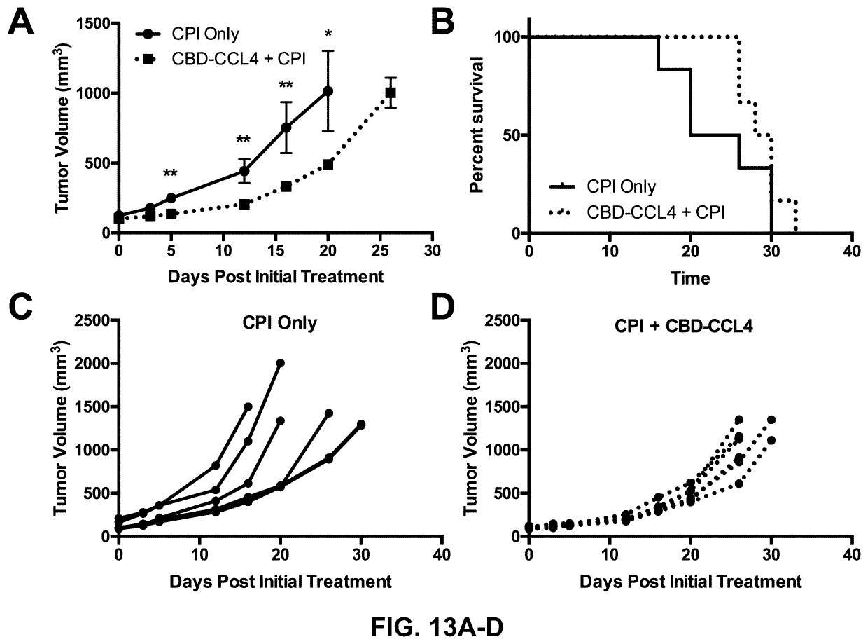

Finally, the efficacy of combination therapy in a spontaneous breast cancer model was investigated. To this point, antitumor efficacy of CBD-CCL4 has been demonstrated in multiple implantable tumor models. While striking, these models develop rapidly, which may impact the leakiness and disordered nature of the tumor microvasculature (Nakamura et al., Bioconjug Chem 27, 2225-38, 2016), making it more amenable to targeted via our collagen-binding approach. Slow developing tumors, on the other hand, may not exhibit this same disordered vasculature (Golombek et al., Adv Drug Deliv Rev 130, 17-38, 2018), limiting the effectiveness of CBD-CCL4 therapy. For this study, female FVB/N-Tg(MMTV-PyVT)634Mul/J (MMTV-PyMT) mice were used, which develop invasive ductal carcinomas in their mammary fat pads around 6-7 weeks after birth (Fantozzi and Christofori, Breast Cancer Res 8, 212, 2006; Guy et al., Mol Cell Biol 12, 954-61, 1992). This model is also histologically similar to human breast cancers (Lin et al., Am J Pathol 163, 2113-26, 2003), making it a suitable model to demonstrate the translational potential of CBD-CCL4 combination therapy. Once again, CBD-CCL4 in combination with CPI therapy slowed tumor growth relative to CPI therapy alone (FIG. 13). While survival results were not significantly different, median survival time did increase to 29 days following initial therapy for the combination therapy, compared to 23 days for CPI therapy alone. These results demonstrate that CBD targeting can also be applied to spontaneously developing tumors in addition to implantable tumor models, highlighting the translational potential of this approach. Further optimization of dosing regimens may further improve the benefit afforded by CBD-CCL4 combination therapy.

The inventors have demonstrated a novel method for enhancing the efficacy of CPI immunotherapy (e.g., αPD-1, αPD-L1, and αCTLA-4) through the recruitment of CD103⁺ DCs to the tumor microenvironment using the chemokine CCL4. CBD-CCL4 in combination with CPI therapy, but not WT CCL4 or CPI therapy alone, significantly enhanced the immune infiltrates in both B16F10 and EMT6 tumors and slowed tumor growth. Detailed characterization of the tumor immune cell composition confirmed that significant increases of CD103⁺ DCs and CD8⁺ T cells occurred following CBD-CCL4 combination therapy. This method is amenable to combination with multiple CPI antibody therapies and can be applied to multiple tumor types, highlighting its significant potential for clinical translation for improved cancer immunotherapies.

### B. Materials and Methods

Production and purification of recombinant WWF A3 domain-(CL4 fusion protein. The sequences encoding for the fusion of human vWF A3 domain residues Cys1670-Gly1874 (907-1111 of mature vWF), a (GGGS)₂ linker and murine CCL4 was synthesized and cloned into the pcDNA3.1(+) CMV-driven mammalian expression vector by Genscript. A sequence encoding for the 6× His-tag was added at the N-terminus for downstream purification of the recombinant protein. Suspension-adapted HEK-293F cells were maintained in serum-free FreeStyle293 Expression Medium™ (Gibco). Protein production was performed according to previous protocols (Ishihara et al., Sci Transl Med, 2019; Ishihara et al., Nat Commun 9, 2163, 2018). Briefly, on the day of transfection, cells were transferred into fresh medium at a density of 1×10⁶ cells/mL. 1 μg/mL plasmid DNA was mixed with 2 μg/ml linear 25 kDa polyethylenimine (Polysciences) diluted in OptiPRO™ SFM media (Thermo Fisher), incubated for 20 min, and added dropwise to the cells (4% v/v final concentration). The culture flask was agitated in a humidified orbital shaking incubator at 135 rpm at 37° C. in the presence of 5% CO₂. 6 days after transfection, the cell culture medium was collected, centrifuged, and filtered through a 0.22 μm filter. Culture media was loaded into a HisTrap™ HP 5 mL column (GE Healthcare), using an ÄKTA pure 25 (GE Healthcare). After washing the column with wash buffer (20 mM imidazole, 20 mM NaH₂PO₄, 0.5 M NaCl, pH 7.4), protein was eluted with a gradient of 500 mM imidazole (in 20 mM NaH₂PO₄, 0.5 M NaCl, pH 7.4). The eluted protein was further purified with size exclusion chromatography using a HiLoad™ Superdex 200PG column (GE Healthcare). All purification steps were carried out at 4° C. The expression of CBD-CCL4 was determined by western blotting using anti-His tag antibody (BioLegend, Clone J099B12), and the proteins were verified as >90% pure by SDS-PAGE. Native form murine CCL4 protein was purchased commercially from Biolegend.

Murine CCL4-
(SEQ ID NO: 25)
APMGSDPPTSCCFSYTSRQLHRSFVMDYYETSSLCSKPAVVFLTKRGRQI
CANPSEPWVTEYMSDLELN
Human vWF A3 domain and murine CCL4 fusion
protein-
(SEQ ID NO: 47)
CSQPLDVILLLDGSSSFPASYFDEMKSFAKAFISKANIGPRLTQVSVLQY
GSITTIDVPWNVVPEKAHLLSLVDVMQREGGPSQIGDALGFAVRYLTSEM
HGARPGASKAVVILVTDVSVDSVDAAADAARSNRVTVFPIGIGDRYDAAQ
LRILAGPAGDSNVVKLQRIEDLPTMVTLGNSFLHKLCSGFVRIGGGGSGG
GGSAPMGSDPPTSCCFSYTSRQLHRSFVMDYYETSSLCSKPAVVFLTKRG
RQICANPSEPWVTEYMSDLELNHHHHHH

SDS-PAGE analysis of protein molecular weight and purity. The measurement was performed as described previously (Ishihara et al., Sci Transl Med 9, 2017). SDS-PAGE was performed on 4-20% gradient gels (Bio-Rad) after CCL4 or CBD-CCL4 was reduced with β-mercaptoethanol. After electrophoresis, gels were stained with SimplyBlue SafeStain™ (Thermo Fisher Scientific) according to the manufacturer's recommendations. Gel images were acquired with the ChemiDoc XRS+ system™ (Bio-Rad).

CBD-CCL4 collagen binding measurements using SPR. SPR measurements were made with a Biacore X100 SPR system™ (GE Healthcare). Collagen I or collagen III was immobilized via amine coupling on a CM5 chip (GE Healthcare) for ˜1000 resonance units (RU) according to the manufacturer's instructions. CBD-CCL4 was flowed for 90 s (for collagen I) and for 30 s (for collagen III) at increasing concentrations in the running buffer at 30 μL/min. The sensor chip was regenerated with 50 mM NaOH for each cycle. Specific binding of CBD-CCL4 to collagen was calculated automatically using the response to a non-functionalized channel as a reference. Binding curves were fitted using BIAevalution™ software (GE Healthcare). CBD-CCL4 binding results were fitted with Langmuir binding kinetics (1:1 binding).

GPCR calcium flux signaling assay. GPCR signaling following interaction with native form CCL4 or CBD-CCL4 was analyzed using a calcium flux assay (FLUOFORTE™ Calcium Assay Kit, Enzo Life Sciences). The assay was performed according to the manufacturer's protocol with slight modifications. Reagents were reconstituted, mixed as directed, and brought to room temperature before use. 24 h prior, 1.5×10⁵ ThP1 human monocytes, known to express CCR5 (Gouwy et al., Eur J Immunol 41, 963-73, 2011), were plated in each well of a tissue-culture treated 96 well round bottom plate. On the day of the assay, cells were spun down at 2000 RPM for 2 min, media was removed, and cells were resuspended in 100 μL of assay buffer. Cells were subsequently incubated for 45 min at 37° C. and then for 15 min at room temperature prior to assay. Samples were then prepared separately in PBS in triplicate and then diluted 1:4 upon addition to the cells to give the indicated molar concentration of CCL4. After addition of the compound, samples were mixed several times with a multichannel pipette to ensure thorough mixing, after which they were transferred to a black-walled clear bottom 96 well plate. Calcium signaling was then measured using a Cytation™ 3 multi-mode plate reader (BioTek) at an excitation wavelength of 490 nm and an emission wavelength of 525 nm, using bottom-read optics with the gain set at 100. EC₅₀ values were calculated using a non-linear dose-response curve fitting model comparing log (test compound) vs. response in GraphPad Prism™.

Tumor cell line culture and maintenance and animal sourcing. All cells were maintained in a humidified incubator at 37° C. and 5% CO₂. Cells were routinely passaged using TrypLE Express™ (Thermo Fisher) once they reached 80-90% confluence. B16F10 melanoma and MC38 colon carcinoma cells were maintained in Dulbecco's Modified Eagle Medium™ (DMEM) supplemented with 10% (v/v) fetal bovine serum (FBS, Certified, US Origin, Heat-Inactivated, Gibco) and 500 U/mL (1% v/v) penicillin-streptomycin (P/S, Gibco). EMT6 breast cancer, CT26 colon carcinoma, and ThP1 monocyte cells were maintained in RPMI1640 supplemented with 10% (v/v) FBS and 1% (v/v) P/S. All cells were confirmed to be mycoplasma free using a MycoAlert Plus™ mycoplasma assay (Lonza). Female C57BL/6 and Balb/C mice, age 8 wk to 12 wk, were obtained from the Jackson Laboratories. Female FVB/N-Tg(MMTV-PyVT)634Mul/J (MMTV-PyMT) mice, age 4 wk to 6 wk, were obtained from the Jackson Laboratories. MMTV-PyMT mice were inspected weekly until tumors were palpable in at least 4mammary fat pads. All mice were acclimated in their cages for 72 h prior to use. Treatments were randomized within cages to minimize cage-specific treatment effects. All animal experiments were performed with approval and according to the policies of the Institutional Animal Care and Use Committee at The University of Chicago.

Blood plasma half-life characterization. 5×10⁵ B16F10 melanoma cells were injected intradermally on the left side of the back of each mouse. WT CCL4 and CBD-CCL4 were fluorescently labeled using Dylight™ 800-NHS (Thermo Fisher), and unreacted dye was removed by a Zebaspin™ spin column (Thermo Fisher) according to the manufacturer's instruction. After 4 days, mice were injected with 25 μg WT CCL4-DyLight™ 800 or the molar equivalent (25 μg CCL4 basis, or 93 μg total protein) of CBD-CCL4-DyLight™ 800 via i.v. injection. Blood samples were collected into EDTA-containing tubes via facial bleed at 1 min, 5 min, 10 min, and 30 min after administration. Samples were then centrifuged at 2000 rpm for 5 min to collect plasma. Concentrations of CCL4 in plasma were measured using a LI-COR™ Infrared Odyssey™ Imager, and concentrations were calculated from a standard dilution series of labeled WT CCL4 or CBD-CCL4.Blood plasma half-life was determined using a one-phase decay model using GraphPad Prism™ software (Version 7).

Biodistribution analysis in EMT6 tumor-bearing mice. WT CCL4 or CBD-CCL4protein was fluorescently labeled using DyLight™ 647 NHS ester (Thermo Fisher), and unreacted dye was removed by a Zebaspin™ spin column (Thermo Fisher) according to the manufacturer's instruction. A total of 5×10⁵ EMT6 cells re-suspended in 50 μL of PBS were injected subcutaneously into the mammary fat pad on the right side of each Balb/C mouse. When the tumor reached approximately 500 mm³, 25 μg DyLight™ 647 labeled CCL4 or 25 μg (6.7 μg CCL4 basis) DyLight™ 647-labeled CBD-CCL4 was injected i.v. 30 min after injection, mice were sacrificed and tumors were extracted and imaged with the Xenogen IVIS Imaging System™ 100 (Xenogen) under the following conditions: f/stop: 2; optical filter excitation 640 nm; emission 670 nm; exposure time: 0.5 sec; small binning. CCL4 concentration in each tumor was calculated based on a standard dilution series of WT CCL4 or CBD-CCL4 labeled with DyLight™ 647 and normalized to the weight of the tumor.

Anti-tumor efficacy in B16F10 melanoma. A total of 5×10⁵ B16F10 cells resuspended in 50 μL PBS were inoculated intradermally on the left side of the back of each C57BL/6 mouse. After 4 d (or 7 d for established tumor treatment study), mice were injected with WT CCL4 (25 μg given via i.v. injection) or molar equivalent CBD-CCL4 (25 μg CCL4 basis, or 93 μg CBD-CCL4, given via i.v. injection) in combination with CPI antibody therapy consisting of 100 μg each αPD-L1 and αCTLA4 given via i.p. injection. CPI therapy alone was administered as control. Tumors were measured with a digital caliper starting 4 days after tumor inoculation, and volumes were calculated as ellipsoids, where V=4/3×π×depth/2×width/2×height/2. Mice were sacrificed when tumor volume exceeded 500 mm³ or when early removal criteria were met due to poor health of the mice. In the case of cell infiltrate analysis, mice were sacrificed 10 d after tumor inoculation.

Anti-tumor efficacy in EMT6 breast cancer. A total of 5×10⁵ EMT6 cells re-suspended in 50 μL of PBS were injected subcutaneously into the mammary fat pad on the right side of each Balb/C mouse. 6 d and 9 d after tumor inoculation, tumors were administered with WT CCL4 (25 μg given via i.v. injection) or molar equivalent CBD-CCL4 (25 μg CCL4 basis, or 93 μg CBD-CCL4, given via i.v. injection) in combination with CPI antibody therapy consisting of 100 μg each αPD-L1 and αCTLA4 given via i.p. injection. CPI therapy alone was administered as control. Tumors were measured with a digital caliper starting 4 days after tumor inoculation as described above. Mice were sacrificed 10 d after tumor inoculation to evaluate immune cell infiltration.

Anti-tumor efficacy in CT26 and MC38 colon carcinoma. A total of 5×10⁵ CT26 or MC38 cells re-suspended in 50 μL of PBS were inoculated intradermally on the left side of the back of each Balb/c (for CT26) or C57BL/6 (for MC38) mouse. After 5 days, mice were injected with unmodified CCL4 (25 μg given via i.v. injection) or molar equivalent CBD-CCL4 (25 μg CCL4 basis, or 93 μg CBD-CCL4, given via i.v. injection) in combination with 100 μg αPD-1 antibody therapy given via i.p. injection. Tumors were measured with a digital caliper starting 5 days after tumor inoculation as described above. Mice were sacrificed when tumor volume exceeded 500 mm³.

Anti-tumor efficacy in MMTV-PyMT breast cancer. Once total tumor volume reached approximately 100 mm³, mice were treated with CBD-CCL4 (25 μg CCL4 basis, or 93 μg CBD-CCL4, given via i.v. injection) in combination with CPI antibody therapy consisting of 100 μg each αPD-L1 and αCTLA4 given via i.p. injection. Identical treatments were given 7 d and 14 d after initial therapy. Tumors were measured bi-weekly with a digital caliper as described above, and mice were sacrificed once tumor volume exceeded 1000 mm³ or mice experienced adverse effects due to tumor burden.

Tissue and single-cell preparation for immune cell analysis. Both B16F10 and EMT6 tumors were harvested 10 d after initial tumor inoculation. All cell isolation proceudres were adapted from previously reported methods (Ishihara et al., Sci Transl Med 9, 2017; Ishihara et al., Sci Transl Med, 2019). Tumors were minced into small pieces, after which enzymatic digestion consisting of 2 mg/mL collagenase D and 40 μg/mL DNase I (Roche) in DMEM containing 2% FBS was performed for 30 min at 37° C. under gentle agitation. Single-cell suspensions were obtained by gently disrupting the enzyme-treated tumor through a 70-μm cell strainer. Red blood cells were lysed with ACK lysing buffer (Quality Biological), after which cells were centrifuged and resuspended in flow cytometry staining buffer consisting of PBS containing 2% FBS for downstream analysis.

Flow cytometry analysis and antibodies used. Single cell suspensions from tumors were prepared as described above. Antibodies against the following molecules were used in all experiments: anti-mouse CD3 (145-2C11, BD Biosciences), anti-mouse CD4 (RM4-5, BD Biosciences), anti-mouse CD8α (53-6.7, BD Biosciences), anti-mouse CD25 (PC61, BD Biosciences), anti-mouse CD45 (30-F11, Biolegend) anti-mouse CD44 (IM7, Biolegend), anti-mouse CD62L (MEL-14, BD Biosciences), anti-mouse PD-1 (29F.1A12, Biolegend), anti-mouse NK1.1 (PK136, Biolegend), anti-mouse Foxp3 (MF23, BD Biosciences), anti-mouse F4/80 (BM8, Biolegend), anti-mouse MHCII (M5/114.15.2, BioLegend), anti-mouse CD11b (M1/70, BioLegend), anti-mouse CD11c (N418, Biolegend), anti-mouse CD19 (1D3, BD Biosciences), anti-mouse Gr-1 (RB6-8C5, Biolegend) and anti-mouse CD103 (M290, BD Biosciences) Live/dead cell discrimination was performed using Fixable Viability Dye eFluor 455 (eBioscience) according to the manufacturer's instructions; an F_{C} receptor blocking step (anti-mouse CD16/32, clone 93, Biolegend) was also included to minimize non-specific antibody binding. Surface staining was carried out on ice for 20 min, and intracellular staining was performed using the FoxP3-transcription factor staining kit according to manufacturer's instructions (eBioscience). Otherwise, samples were fixed in 2% paraformaldehyde in PBS. All flow cytometric analyses were done using a Fortessa™ (BD Biosciences) flow cytometer and analyzed using FlowJo™ software (Tree Star).

Statistical Analysis. The statistical significance between treatment groups was assessed using Prism™ software (v7, GraphPad). For multiple comparisons, ANOVA followed by Tukey's HSD post hoc test was used when between groups was found to be similar by Brown-Forsythe test. For non-parametric data, Kruskal-Wallis test followed by Dunn's multiple comparison test was used. For comparisons between two groups, a two-tailed Student's 1-test was used. Survival curves were analyzed using the log-rank (Mantel-Cox) test. *p<0.05; **p<0.01; ***p<0.001; ****p<0.0001.

Although certain embodiments have been described above with a certain degree of particularity, or with reference to one or more individual embodiments, those skilled in the art could make numerous alterations to the disclosed embodiments without departing from the scope of this invention. Further, where appropriate, aspects of any of the examples described above may be combined with aspects of any of the other examples described to form further examples having comparable or different properties and addressing the same or different problems. Similarly, it will be understood that the benefits and advantages described above may relate to one embodiment or may relate to several embodiments. Any reference to a patent publication or other publication is a herein a specific incorporation by reference of the disclosure of that publication. The claims are not to be interpreted as including means-plus- or step-plus-function limitations, unless such a limitation is explicitly recited in a given claim using the phrase(s) “means for” or “step for,” respectively.

1. A polypeptide comprising a cytokine polypeptide operatively linked to an extracellular matrix (ECM)-affinity peptide comprising a collagen binding domain, where the ECM-affinity peptide does not specifically bind fibronectin.

2. The polypeptide of claim 1, wherein the ECM-affinity peptide comprises a collagen binding domain of 100 to 350 amino acids of von Willebrand factor (vWF) or decorin.

3. The polypeptide of claim 2, wherein the vWF peptide is a vWF A1 or A3 peptide.

4. The polypeptide of claim 2, wherein the peptide is at least 85% identical to SEQ ID NO:3; SEQ ID NO:11; SEQ ID NO:13; SEQ ID NO:15, or SEQ ID NO:34.

5. The polypeptide of claim 2, wherein the peptide comprises SEQ ID NO:34.

6. The polypeptide of claim 2, wherein the vWF peptide comprises a peptide that is at least 85% identical to SEQ ID NO:3, SEQ ID NO:11, SEQ ID NO:34, or fragments thereof.

7. The polypeptide of claim 6, wherein the vWF peptide comprises SEQ ID NO:3 or SEQ ID NO:11 or SEQ ID NO:34.

8. The polypeptide of claim 6, wherein the vWF peptide consists essentially of SEQ ID NO:3 or SEQ ID NO:11 or SEQ ID NO:34.

9. The polypeptide of claim 1, wherein the peptide comprises a decorin peptide.

10. The polypeptide of claim 9, wherein the peptide comprises 10 to 200 consecutive amino acids of SEQ ID NO:15.

11. The polypeptide of claim 9 or 10, wherein the peptide is at least 85% identical to SEQ ID NO:15.

12. The polypeptide of any one of claims 1-11, wherein the peptide is covalently linked to the cytokine.

13. The polypeptide of any one of claims 1-12, wherein the peptide is crosslinked to the cytokine through a bifunctional crosslinker.

14. The polypeptide of any one of claims 1-13, wherein the cytokine is selected from IL-2, IL-15, IL-15 super agonist, IL-21, IL-12 p35, IL-12 p40, CCL4, CCL21, CXCL9, CXCL10, VEGF-C, IFNα2, IFNβ, XCL-1, or an active fragment thereof.

15. The polypeptide of any one of claims 1-14, wherein the cytokine is at least 85% identical to SEQ ID NO:37, SEQ ID NO:16, SEQ ID NO:17, SEQ ID NO:18, SEQ ID NO:19, SEQ ID NO:20, SEQ ID NO:21, SEQ ID NO:22, SEQ ID NO:23, SEQ ID NO:24, SEQ ID NO:25, SEQ ID NO:26, SEQ ID NO:27, SEQ ID NO:28, SEQ ID NO:29, SEQ ID NO:30, SEQ ID NO:31, SEQ ID NO:32, SEQ ID NO:33, SEQ ID NO:39, SEQ ID NO:40, SEQ ID NO:41, SEQ ID NO:42, SEQ ID NO:43, SEQ ID NO:44, SEQ ID NO:45, or SEQ ID NO:46.

16. The polypeptide of claim 13 or 15, wherein the cytokine is IL-2.

17. The polypeptide of any one of claims 1-16, wherein the ratio of peptide to cytokine is about 1:1 to 10:1.

18. The polypeptide of claim 1, wherein the composition comprises an IL-2 cytokine operatively linked to vWF peptide.

19. A nucleic acid vector comprising a heterologous coding region that encodes a polypeptide of any on of claims 1 to 18.

20. A composition comprising a polypeptide of any one of claims 1 to 18 comprised in a pharmaceutical formulation.

21. The composition of claim 20, further comprising at least one an anti-cancer compound.

22. A method for treating cancer in a subject comprising administering the composition of any one of claims 1-18 to a subject.

23. The method of claim 22, wherein the composition is administered intravascularly, systemically or by intra-tumoral, peri-tumoral, intraarterial, or transcatheter injection.

24. The method of claim 22, wherein intravascularly is intravenously.

25. The method of claim 22 or 23 or 24, wherein the administered dose of the cytokine operatively linked to the peptide is less than the minimum effective dose of the cytokine administered without the peptide.

26. The method of claim 25, wherein the administered dose of the cytokine operatively linked to the peptide is at least 10% less than the minimum effective dose of the cytokine administered without the peptide.

27. The method of any one of claims 22-26, wherein the patient has been previously treated with a cancer immunotherapeutic.

28. The method of any one of claims 22-26, further comprising administering an anti-cancer therapeutic.

29. The method of claim 27, wherein the subject experienced grade two, three, or four side effects from the previous cancer therapeutic.

30. The method of any one of claims 22-29, wherein the subject has been diagnosed with a cancer.

31. The method of any one of claims 22-30, wherein the cancer comprises melanoma, colon cancer, lung cancer, prostate cancer, ovarian cancer, testicular cancer, brain cancer, glioblastoma, pediatric tumors, germ cell tumors, rectal cancer, gastric cancer, esophageal cancer, tracheal cancer, head and neck cancer, pancreatic cancer, liver cancer, breast cancer, cervical cancer, and vulvar cancer.

32. The method of claim 31, wherein the cancer is melanoma or colon cancer.

33. The method of any one of claims 22-30, wherein the cancer is non-hematological.

34. The method of any one of claims 22-33, wherein the cancer comprises a solid tumor.

35. The method of any one of claims 22-34, wherein the method further comprises administration of an additional cancer therapy.

36. The method of claim 35, wherein the additional cancer therapy comprises radiation, vaccination, chemotherapy, adoptive T-cell therapy, cytokine therapy, anti-CD47 antibodies, anti-GD2 antibodies, or immunologic adjuvants.

37. The method of claim 35, wherein the polypeptide of any one of claims 1-18 is administered before, after, during, before and during, before and after, or during and after administration of anti-cacner therapeutic.

38. The method of claim 35 or 36, wherein the additional cancer therapy comprises one or more of MUC-1 inhibitors, CD40 activators, IDO inhibitors, and OX86 agonists.

39. The method of claim 38, wherein the additional cancer therapy comprises one or more of indoximod, GDC-0919, 1-methyl-D-tryptophan, norharmane hydrochloride, norharmane, CAY10581, INCB024360, and 2-benzyl-2-thiopseudourea hydrochloride.

40. The method of any one of claim 22-39, wherein the method further comprises administration of a second polypeptide comprising a cytokine operatively linked to a an extracellular matrix (ECM)-affinity peptide.

41. The method of claim 40, wherein a first and second polypeptide are administered together.

42. The method of claim 41, wherein the first and second polypeptide are co-formulated.

43. A method of making a polypeptide of any one of claims 1 to 18 comprising expressing the polypeptide in a host cell and isolating the polypeptide expressed by the host cell.

44. The method of claim 43, wherein the host cell is a mammalian host cell.

45. The method of claim 43, wherein isolating the polypeptide comprises affinity column purification and/or size-exclusion column purification.
